(19) United States
(12) Patent Application Publication
(10) Pub. No.: US 2004/0235952 A1
(43) Pub. Date: Nov. 25, 2004

(54) Inhibitors of severe acute respiratory syndrome (SARS) 3C-like proteinase

(75) Inventors: Shella Fuhrman, San Diego, CA (US); David A. Matthews, Encinitas, CA (US); Amy Karen Patick, Escondido, CA (US); Paul Rejto, Carlsbad, CA (US)

Correspondence Address: AGOURON PHARMACEUTICALS, INC., 10350 NORTH TORREY PINES ROAD, LA JOLLA, CA, 92037, US

(73) Assignee: Agouron Pharmaceuticals, Inc.

(21) Appl. No.: 10/833,685

(22) Filed: Apr. 27, 2004

Related U.S. Application Data
(60) Provisional application No. 60/468,197, filed on May 5, 2003.
(60) Provisional application No. 60/468,504, filed on May 6, 2003.
(60) Provisional application No. 60/469,065, filed on May 7, 2003.

(51) Int. Cl. A61K 31/195

(52) U.S. Cl. 514/563

(57) ABSTRACT

The invention relates to methods of inhibiting SARS-related coronavirus viral replication activity comprising contacting a SARS-related coronavirus protease with a therapeutically effective amount of a rhinovirus protease inhibitor, and compositions comprising the same.

### BACKGROUND OF THE INVENTION

The invention relates to methods of inhibiting Severe Acute Respiratory Syndrome (SARS) viral replication activity comprising contacting a SARS-related coronavirus 3C-like proteinase with a therapeutically effective amount of a rhinovirus 3C protease inhibitor. The invention further relates to pharmaceutical compositions containing the rhinovirus 3C proteinase (RVP) inhibitor in a mammal by administering effective amounts of such rhinovirus 3C proteinase inhibitor.

A worldwide outbreak of Severe Acute Respiratory Syndrome-related virus(es) (SARS) has been associated with exposures originating from a single ill health care worker from Guangdong Province, China. Recently, the causative agent has been identified as a novel coronavirus. There is a need in the art for an effective treatment for the SARS-related coronavirus.

In particular, recent evidence strongly implicates a new coronavirus as the causative agent of SARS (CDC). Coronavirus replication and transcription function is encoded by the so-called “replicase” gene (Thiel, Herold et al. 2001), which consists of two overlapping polyproteins that are extensively processed by viral proteases. The C-proximal region is processed at eleven conserved interdomain junctions by the coronavirus main or “3C-like” protease (Ziebuhr, Snijder et al. 2000). The name “3C-like” protease derives from certain similarities between the coronavirus enzyme and the well-known picornavirus 3C proteases (Gorbalenya, Koonin et al. 1989). These include substrate preferences, use of cysteine as an active site nucleophile in catalysis, and similarities in their putative overall polypeptide folds. Very recently Hilgenfeld and colleagues published a high-resolution X-ray structure of the porcine transmissible gastroenteritis coronavirus main protease (Anand, Palm et al. 2002). Atomic coordinates are available through the Protein Data Bank under accession code 1LVO.

For almost 10 years, research organizations like Pfizer-La Jolla have been engaged in an effort to discover and develop drugs with utility for treating the common cold by targeting a key enzyme in rhinovirus replication, namely the 3C protease (Matthews, Smith et al. 1994). The picornaviruses are a family of tiny non-enveloped positive-stranded RNA-containing viruses that infect humans and other animals. These viruses include the human rhinoviruses, human polioviruses, human coxsackieviruses, human echoviruses, human and bovine enteroviruses, encephalomyocarditis viruses, meningitis virus, foot and mouth viruses, hepatitis A virus, and others. Picomaviral infections may be treated by inhibiting the proteolytic 3C enzymes. These enzymes are required for the natural maturation of the picornaviruses. They are responsible for the autocatalytic cleavage of the genomic, large polyprotein into the essential viral proteins. Members of the 3C protease family are cysteine proteases, where the sulfhydryl group most often cleaves the glutamine-glycine amide bond. Inhibition of 3C proteases is believed to block proteolytic cleavage of the polyprotein, which in tum can retard the maturation and replication of the viruses by interfering with viral particle production.

Potent, non-toxic agents with broad-spectrum activity against multiple rhinovirus serotypes have been identified (Patick, Binford et al. 1999). Significant catalytic and structural similarities between rhinovirus 3C protease and coronavirus “3C-like” main protease suggest that selected inhibitors of rhinovirus 3C protease are useful against coronavirus main protease.

### SUMMARY OF THE INVENTION

The present invention provides methods of inhibiting the activity of a coronavirus 3C protease (also known as proteinase), comprising contacting the coronavirus 3C protease with an effective amount of a picornavirus inhibitor compound, and preferably a rhinovirus inhibitor compound or agent.

The present invention provides a novel method of interfering with or preventing SARS viral replication activity comprising contacting a SARS protease with a therapeutically effective amount of a rhinovirus protease inhibitor.

In one embodiment of the present invention, the SARS coronavirus 3C-like protease inhibitor is administered orally or intravenously.

The present invention also provides a method of treating a condition that is mediated by coronavirus 3C-like protease activity in a patient by administering to said patient a pharmaceutically effective amount of a rhinovirus protease inhibitor.

The present invention also provides a method of targeting SARS inhibition as a means of treating indications caused by SARS-related viral infections.

The present invention also provides a method of targeting viral or cellular targets identified by using rhinovirus inhibitors against SARS coronavirus 3C-like protease for treating indications caused by SARS-related viral infections.

The present invention also provides a method of identifying cellular or viral pathways interfering with the functioning of the members of which could be used for treating indications caused by SARS infections by administering a rhinovirus protease inhibitor.

The present invention also provides a method of using rhinovirus protease inhibitors as tools for understanding mechanism of action of other SARS inhibitors.

The present invention also provides a method of using rhinovirus protease inhibitors for carrying out gene profiling experiments for monitoring the up or down regulation of genes for the purposed of identifying inhibitors for treating indications caused by SARS infections.

The present invention further provides a pharmaceutical composition for the treatment of SARS in a mammal containing an amount of a rhinovirus protease inhibitor that is effective in treating SARS and a pharmaceutically acceptable carrier.

In another aspect, the invention provides a method of interfering with or preventing SARS related coronavirus viral replication activity comprising contacting a SARS related coronavirus protease with a therapeutically effective amount of a rhinovirus 3C protease inhibitor of formula I:

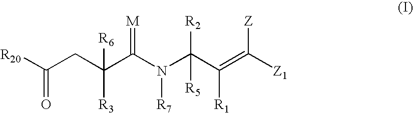

wherein

M is O or S;

R₁ is H, F, an alkyl group, OH, SH, or an O-alkyl group;

R₂ and R₅ are independently selected from H,

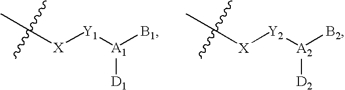

or an alkyl group, wherein said alkyl group is different from

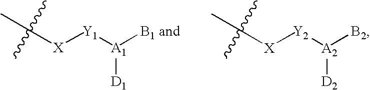

with the proviso that at least one of R₂ or R₅ must be

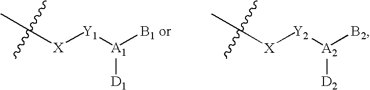

and wherein, when R₂ or R₅ is

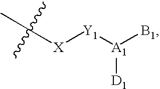

X is ═CH or ═CF and Y₁ is ═CH or ═CF,

or X and Y₁ together with Q′ form a three-membered ring in which Q′ is —C(R₁₀)(R₁₁)— or —O—, X is —CH— or —CF—, and Y₁ is —CH—, —CF—, or —C(alkyl)—, where R₁₀ and R₁₁ independently are H, a halogen, or an alkyl group, or, together with the carbon atom to which they are attached, form a cycloalkyl group or a heterocycloalkyl group,

or X is —CH₂—, —CF₂—, —CHF—, or —S—, and Y₁ is —O—, —S—, —NR₁₂—, —C(R₁₃)(R₁₄)—, —C(O)—, —C(S)—, or —C(CR₁₃R₁₄)—,

wherein R₁₂ is H or alkyl, and R₁₃ and R₁₄ independently are H, F, or an alkyl group, or, together with the atoms to which they are bonded, form a cycloalkyl group or a heterocycloalkyl group;

A₁ is C, CH, CF, S, P, Se, N, NR₁₅, S(O), Se(O), P—OR₁₅, or P—NR₁₅R₁₆, wherein R₁₅ and R₁₆ independently are an alkyl group, a cycloalkyl group, a heterocycloalkyl group, an aryl group, or a heteroaryl group, or, together with the atom to which they are bonded, form a heterocycloalkyl group;

D₁ is a moiety with a lone pair of electrons capable of forming a hydrogen bond; and

B₁ is H, F, an alkyl group, a cycloalkyl group, a heterocycloalkyl group, an aryl group, a heteroaryl group, —OR₁₇, —SR₁₇, —NR₁₇R₁₈, —NR₁₉NR₁₇R₁₈, or —NR₁₇OR₁₈,

wherein R₁₇, R₁₈, and R₁₉ independently are H, an alkyl group, a cycloalkyl group, a heterocycloalkyl group, an aryl group, a heteroaryl group, or an acyl group;

and with the provisos that when D₁ is the moiety □N with a lone pair of electrons capable of forming a hydrogen bond, B₁ does not exist; and when A₁ is an sp³ carbon, B₁ is not —NR₁₇R₁₈ when D₁ is the moiety —NR₂₅R₂₆ with a lone pair of electrons capable of forming a hydrogen bond, wherein R₂₅ and R₂₆ are independently H, an alkyl group, a cycloalkyl group, a heterocycloalkyl group, an aryl group, or a heteroaryl group;

and wherein D₁-A₁-B₁ optionally forms a nitro group where A₁ is N;

and further wherein, when R₂ or R₅ is

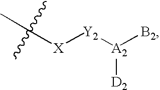

X is ═CH or ═CF and Y₂ is ═C, ═CH, or ═CF,

or X and Y₂ together with Q′ form a three-membered ring in which Q′ is —C(R₁₀)(R₁₁)— or —O—, X is —CH— or —CF—, and Y₂ is —CH—, —CF—, or —C(alkyl)—, where R₁₀ and R₁₁ independently are H, a halogen, or an alkyl group, or, together with the carbon atom to which they are attached, form a cycloalkyl group or a heterocycloalkyl group,

or X is —CH₂—, —CF₂—, —CHF—, or —S—, and Y₂ is —O—, —S—, —N(R′₁₂)—, —C(O)—, —C(R′₁₃)(R′₁₄)—, —C(S)—, or —C(CR′₁₃R′₁₄)—,

wherein R′₁₂ is H, an alkyl group, a cycloalkyl group, a heterocycloalkyl group, an aryl group, a heteroaryl group, —OR′₁₃, —NR′₁₃R′₁₄, —C(O)—R′₁₃, —SO₂R′₁₃, or —C(S)R′₁₃, and R′₁₃ and R′₁₄, independently are H, F, or an alkyl group, a cycloalkyl group, a heterocycloalkyl group, an aryl group, or a heteroaryl group, or, together with the atom to which they are attached, form a cycloalkyl group or a heterocycloalkyl group;

A₂ is C, CH, CF, S, P, Se, N, NR₁₅, S(O), Se(O), P—OR₁₅, or P—NR₁₅R₁₆, wherein R₁₅ and R₁₆ independently are an alkyl group, a cycloalkyl group, a heterocycloalkyl group, an aryl group, or a heteroaryl group, or, together with the atom to which they are bonded, form a heterocycloalkyl group;

D₂ is a moiety with a lone pair of electrons capable of forming a hydrogen bond; and

B₂ is H, F, an alkyl group, a cycloalkyl group, a heterocycloalkyl group, an aryl group, a heteroaryl group, —OR₁₇, —SR₁₇, —NR₁₇R₁₈, —NR₁₉NR₁₇R₁₈, or —NR₁₇OR₁₈,

wherein R₁₇, R₁₈, and R₁₉ independently are H, an alkyl group, a cycloalkyl group, a heterocycloalkyl group, an aryl group, a heteroaryl group, or an acyl group;

and further wherein any combination of Y₂, A₂, B₂, and D₂ optionally can form a cycloalkyl group, a heterocycloalkyl group, an aryl group, or a heteroaryl group;

R₃ and R₆ are independently H, F, an alkyl group, a cycloalkyl group, a heterocycloalkyl group, an aryl group, a heteroaryl group, —C(O)R₁₇, —OR₁₇, —SR₁₇, —NR₁₇R₁₈, —NR₁₉NR₁₇R₁₈, or —NR₁₇OR₁₈,

wherein R₁₇, R₁₈, and R₁₉ independently are H, an alkyl group, a cycloalkyl group, a heterocycloalkyl group, an aryl group, a heteroaryl group, or an acyl group;

or, R₃ and R₆, together with the carbon atom to which they are attached, form a cycloalkyl group or a heterocycloalkyl group;

R₇ is H, an alkyl group, a cycloalkyl group, a heterocycloalkyl group, an aryl group, a heteroaryl group, —OR₁₇, —SR₁₇, —NR₁₇R₁₈, —NR₁₉NR₁₇R₁₈, or —NR₁₇OR₁₈,

wherein R₁₇, R₁₈, and R₁₉ independently are H, an alkyl group, a cycloalkyl group, a heterocycloalkyl group, an aryl group, a heteroaryl group, or an acyl group;

or R₇, together with R₃ or R₆ and the atoms to which they are attached, forms a heterocycloalkyl group;

R₂₀ is H, OH, or any suitable organic moiety; and

Z and Z₁ are independently H, F, an alkyl group, a cycloalkyl group, a heterocycloalkyl group, an aryl group, a heteroaryl group, —C(O)R₂₁, —CO₂R₂₁, —CN, —C(O)NR₂₁R₂₂, —C(O)NR₂₁OR₂₂, —C(S)R₂₁, —C(S)NR₂₁R₂₂, —NO₂₁, —SOR₂₁, —SO₂R₂₁, —SO₂NR₂₁R₂₂, —SO(NR₂₁)(OR₂₂), —SONR₂₁, —SO₃R₂₁, —PO(OR₂₁)₂, —PO(R₂₁)(R₂₂), —PO(NR₂₁R₂₂)(OR₂₃), PO(NR₂₁R₂₂)(NR₂₃R₂₄), —C(O)NR₂₁NR₂₂R₂₃, or —C(S)NR₂₁NR₂₂R₂₃,

wherein R₂₁, R₂₂, R₂₃, and R₂₄ are independently H, an alkyl group, a cycloalkyl group, a heterocycloalkyl group, an aryl group, a heteroaryl group, an acyl group, or a thioacyl group, or wherein any two of R₂₁, R₂₂, R₂₃, and R₂₄, together with the atom(s) to which they are bonded, form a heterocycloalkyl group;

or Z₁, as defined above, together with R₁, as defined above, and the atoms to which Z₁ and R₁ are bonded, form a cycloalkyl or heterocycloalkyl group,

or Z and Z₁, both as defined above, together with the atoms to which they are bonded, form a cycloalkyl or heterocycloalkyl group;

or a pharmaceutically acceptable prodrug, salt, active metabolite, or solvate thereof; and wherein said compound, or pharmaceutically acceptable prodrug, salt, active metabolite, or solvate thereof, has antipicornaviral activity with an EC₅₀ less than or equal to 10 μM in a HI-HeLa cell culture assay.

In another aspect, the invention provides a method of interfering with or preventing SARS related coronavirus viral replication activity comprising contacting a SARS related coronavirus protease with a therapeutically effective amount of a rhinovirus 3C protease inhibitor of formula II:

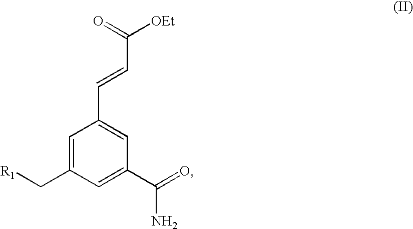

wherein R₁ is:

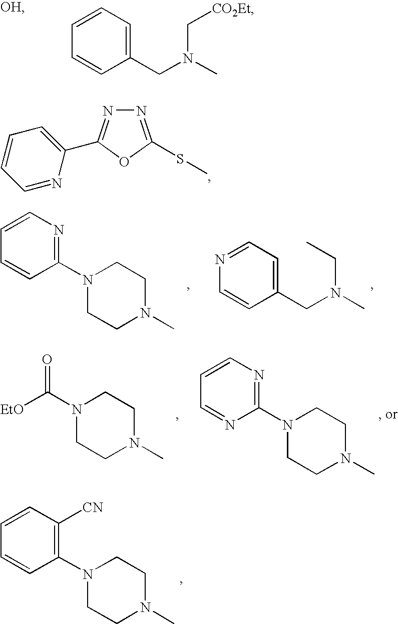

or a pharmaceutically acceptable salt, solvate, prodrug, or pharmaceutically active metabolite thereof.

In still another aspect, the invention provides a method of interfering with or preventing SARS related coronavirus viral replication activity comprising contacting a SARS related coronavirus protease with a therapeutically effective amount of a rhinovirus 3C protease inhibitor of formula IIA:

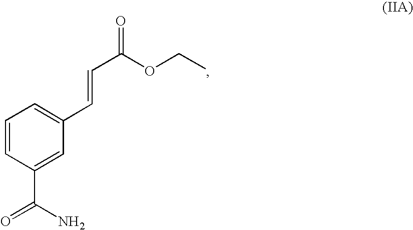

or a pharmaceutically acceptable salt, solvate, prodrug, or pharmaceutically active metabolite thereof.

In yet another aspect, the invention provides a method of interfering with or preventing SARS related coronavirus viral replication activity comprising contacting a SARS related coronavirus protease with a therapeutically effective amount of a rhinovirus 3C protease inhibitor of formula IIB:

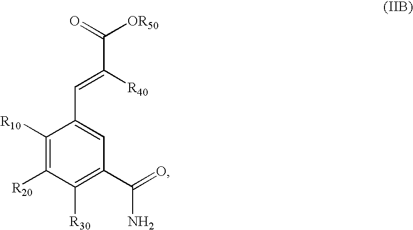

wherein:

R₁₀ is H or CH₃;

R₂₀ is H, OH, CH₂OH, or OCH₂Ph;

R₃₀ is H, OH, or OCH₂Ph;

R₄₀ is H or CN; and

R₅₀ is CH₂CH₃, CH₃, CH₂Ph, CH₂CH₂Ph, CH₂CH₂OH, or CH₂(2-pyridyl);

or a pharmaceutically acceptable salt, solvate, prodrug, or pharmaceutically active metabolite thereof.

In another aspect, the invention provides a method of interfering with or preventing SARS related coronavirus viral replication activity comprising contacting a SARS related coronavirus protease with a therapeutically effective amount of a rhinovirus 3C protease inhibitor of formula IIC:

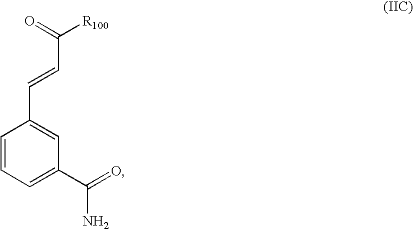

wherein R₁₀₀ is CH₃, phenyl, Ph(4-NCH₃), Ph(4-OCH₃), 2-pyridyl, or 2-furyl;

or a pharmaceutically acceptable salt, solvate, prodrug, or pharmaceutically active metabolite thereof. In another aspect, the invention provides a method of interfering with or preventing SARS related coronavirus viral replication activity comprising contacting a SARS related coronavirus protease with a therapeutically effective amount of a rhinovirus 3C protease inhibitor of formula

In another aspect, the invention provides a method of interfering with or preventing SARS related coronavirus viral replication activity comprising contacting a SARS related coronavirus protease with a therapeutically effective amount of a rhinovirus 3C protease inhibitor of formula III:

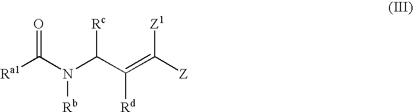

wherein:

R^{a1} is a cycloalkyl, heterocycloalkyl, aryl or heteroaryl group, provided that R^{a1} is not a substituted pyrrolidinyl, where the cycloalkyl, heterocycloalkyl, aryl or heteroaryl group is unsubstituted or substituted with one or more suitable substituents;

R^{c} is a substituent having the formula:

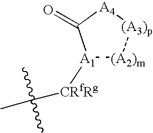

wherein:

R^{f} and R^{g} are each independently H or lower alkyl;

m is 0 or 1;

p is an integer of from 0 to 5;

A₁ is CH or N;

when p is 1, 2, 3, 4, or 5, A₂ is C(R^{h})(Rⁱ), N(R^{j}), S, S(O), S(O)₂, or O, and when p is 0, A₂ is C(R^{h})(Rⁱ)(R^{j}), N(Rⁱ)(R^{j}), S(Rⁱ), S(O)(Rⁱ), S(O)₂(Rⁱ), or O(Rⁱ), where each R^{h}, Rⁱ and R^{j} is independently H or a lower alkyl group;

each A₃ present is independently C(R^{h})(Rⁱ), N(R^{j}), S, S(O), S(O)₂, or O; where each R^{h}, Rⁱ and R^{j} is independently H or lower alkyl;

when p is 1, 2, 3, 4, or 5, A₄ is N(R^{k}), C(R^{h})(Rⁱ), or O; and when p is 0, A₄ is N(R^{k})(Rⁱ), C(R^{h})(Rⁱ)(R^{j}), and O(Rⁱ), where each R^{h}, Rⁱ and R^{j} is independently H or lower alkyl, each R^{k} is H, alkyl, aryl, or acyl, and each R^{l} is H, alkyl, or aryl;

provided that no more than two heteroatoms occur consecutively in the above-depicted ring formed by A₁, (A₂)ₘ, (A₃)ₚ, A₄, and C═O, where each dotted line in the ring depicts a single bond when A₂ is present and a hydrogen atom when A₂ is absent;

R^{d} is H, halogen, hydroxyl or an alkyl, alkoxy or alkylthio group, where the alkyl, alkoxy or alkylthio group is unsubstituted or substituted with one or more suitable substituents;

R^{b} is H or an alkyl group, unsubstituted or substituted with one or more suitable substituents;

Z and Z¹ are each independently H, F, an alkyl, cycloalkyl, heterocycloalkyl, aryl or heteroaryl group, where the alkyl, cycloalkyl, heterocycloalkyl, aryl or heteroaryl group is unsubstituted or substituted with one or more suitable substituents, —C(O)Rⁿ —CO₂Rⁿ —CN, —C(O)NRⁿR^{o}, —C(O)NRⁿOR^{o}, —C(S)Rⁿ, —C(S)ORⁿ —C(S)NRⁿR^{o}, —C(═NRⁿ)R^{o}, —C(═NRⁿ)OR^{o}, —NO₂, —SOR^{o}, —SO₂Rⁿ, —SO₂NRⁿR^{o}, —SO₂(NRⁿ)(OR^{o}), —SONRⁿ, —SO₃Rⁿ, —PO(ORⁿ)₂, —PO(ORⁿ)(OR^{o}), —PO(NRⁿR^{o})(OR^{p}), —PO(NRⁿR^{o})(NR^{p}R^{q}), —C(O)NRⁿNR^{o}R^{p}, —C(S)NRⁿNR^{o}R^{p}, where Rⁿ, R^{o}, R^{p} and R^{q} are each independently H or an alkyl, cycloalkyl, aryl, heterocycloalkyl, acyl or thioacyl group, where the alkyl, cycloalkyl, aryl, heterocycloalkyl, acyl or thioacyl group is unsubstituted or substituted with one or more suitable substituents, or where any two of the Rⁿ, R^{o}, R^{p} and R^{q}, taken together with the atoms to which they are bonded, form a heterocycloalkyl group, which may be optionally substituted,

or Z and R^{d}, together with the atoms to which they are bonded, form a cycloalkyl or heterocycloalkyl group, where Z and R^{d} are as defined above except for moieties that cannot form the cycloalkyl or heterocycloalkyl group,

or Z and Z¹, together with the atoms to which they are bonded, form a cycloalkyl or heterocycloalkyl group, where Z and Z¹ are as defined above (except for moieties that cannot form the cycloalkyl or heterocycloalkyl group);

or a prodrug, pharmaceutically acceptable salt, pharmaceutically active metabolite, or pharmaceutically acceptable solvate thereof.

In another aspect, the invention provides a method of interfering with or preventing SARS related coronavirus viral replication activity comprising contacting a SARS related coronavirus protease with a therapeutically effective amount of a rhinovirus 3C protease inhibitor of formula IIIA:

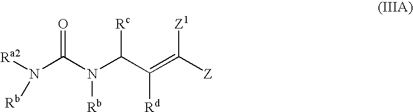

wherein:

R^{a2} is an alkyl, aryl or heteroaryl group, where the alkyl, aryl or heteroaryl group is unsubstituted or substituted with one or more suitable substituents; and

R^{c} is a substituent having the formula:

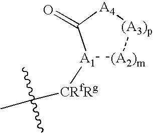

wherein:

R^{f} and R^{g} are each independently H or lower alkyl;

m is 0 or 1;

p is an integer of from 0 to 5;

A₁ is CH or N;

when p is 1, 2, 3, 4, or 5, A₂ is C(R^{h})(Rⁱ), N(R^{j}), S, S(O), S(O)₂, or O, and when p is 0, A₂ is C(R^{h})(Rⁱ)(R^{j}), N(Rⁱ)(R^{j}), S(Rⁱ), S(O)(Rⁱ), S(O)₂(Rⁱ), or O(Rⁱ), where each R^{h}, Rⁱ and R^{j} is independently H or a lower alkyl group;

each A₃ present is independently C(R^{h})(Rⁱ), N(R^{j}), S, S(O), S(O)₂, or O; where each R^{h}, Rⁱ and R^{j} is independently H or lower alkyl;

when p is 1, 2, 3, 4, or 5, A₄ is N(R^{k}), C(R^{h})(Rⁱ), or O; and when p is 0, A₄ is N(R^{k})(Rⁱ), C(R^{h})(Rⁱ)(R^{j}), and O(Rⁱ), where each R^{h}, Rⁱ and R^{j} is independently H or lower alkyl, each R^{k} is H, alkyl, aryl, or acyl, and each R^{l} is H, alkyl, or aryl;

provided that no more than two heteroatoms occur consecutively in the above-depicted ring formed by A₁, (A₂)ₘ, (A₃)ₚ, A₄, and C═O, where each dotted line in the ring depicts a single bond when A₂ is present and a hydrogen atom when A₂ is absent;

R^{d} is H, halogen, hydroxyl or an alkyl, alkoxy or alkylthio group, where the alkyl, alkoxy or alkylthio group is unsubstituted or substituted with one or more suitable substituents;

R^{b} is H or an alkyl group, unsubstituted or substituted with one or more suitable substituents;

Z and Z¹ are each independently H, F, an alkyl, cycloalkyl, heterocycloalkyl, aryl or heteroaryl group, where the alkyl, cycloalkyl, heterocycloalkyl, aryl or heteroaryl group is unsubstituted or substituted with one or more suitable substituents, —C(O)Rⁿ —CO₂Rⁿ —CN, —C(O)NRⁿR^{o}, C(O)NRⁿOR^{o}, —C(S)Rⁿ, —C(S)ORⁿ —C(S)NRⁿR^{o}, —C(═NRⁿ)R^{o}, —C(═NRⁿ)OR^{o}, —NO₂, —SOR^{o}, —SO₂Rⁿ, —SO₂NRⁿR^{o}, —SO₂(NRⁿ)(OR^{o}), —SONRⁿ, —SO₃Rⁿ, —PO(ORⁿ)₂, —PO(ORⁿ)(OR^{o}), —PO(NRⁿR^{o})(OR^{p}), —PO(NRⁿR^{o})(NR^{p}R^{q}), —C(O)NRⁿNR^{o}R^{p}, —C(S)NRⁿNR^{o}R^{p}, where Rⁿ, R^{o}, R^{p} and R^{q} are each independently H or an alkyl, cycloalkyl, aryl, heterocycloalkyl, acyl or thioacyl group, where the alkyl, cycloalkyl, aryl, heterocycloalkyl, acyl or thioacyl group is unsubstituted or substituted with one or more suitable substituents, or where any two of the Rⁿ, R^{o}, R^{p} and R^{q}, taken together with the atoms to which they are bonded, form a heterocycloalkyl group, which may be optionally substituted,

or Z and R^{d}, together with the atoms to which they are bonded, form a cycloalkyl or heterocycloalkyl group, where Z and R^{d} are as defined above except for moieties that cannot form the cycloalkyl or heterocycloalkyl group,

or Z and Z¹, together with the atoms to which they are bonded, form a cycloalkyl or heterocycloalkyl group, where Z and Z¹ are as defined above (except for moieties that cannot form the cycloalkyl or heterocycloalkyl group);

or a prodrug, pharmaceutically acceptable salt, pharmaceutically active metabolite, or pharmaceutically acceptable solvate thereof.

In still another aspect, the invention provides a method of interfering with or preventing SARS related coronavirus viral replication activity comprising contacting a SARS related coronavirus protease with a therapeutically effective amount of a rhinovirus 3C protease inhibitor of formula IIIB:

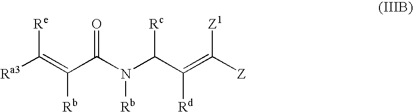

wherein:

R^{a3} is an aryl, heterocycloalkyl, heteroaryl or arylaminocarbonyl group, where the aryl, heterocycloalkyl, heteroaryl or arylaminocarbonyl group is unsubstituted or substituted with one or more suitable substituents; and

R^{c} is a substituent having the formula:

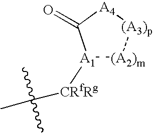

wherein:

R^{f} and R^{g} are each independently H or lower alkyl;

m is 0 or 1;

p is an integer of from 0 to 5;

A₁ is CH or N;

when p is 1, 2, 3, 4, or 5, A₂ is C(R^{h})(Rⁱ), N(R^{j}), S, S(O), S(O)₂, or O, and when p is 0, A₂ is C(R^{h})(Rⁱ)(R^{j}), N(Rⁱ)(R^{j}), S(Rⁱ), S(O)(Rⁱ), S(O)₂(Rⁱ), or O(Rⁱ), where each R^{h}, Rⁱ and R^{j} is independently H or a lower alkyl group;

each A₃ present is independently C(R^{h})(Rⁱ), N(R^{j}), S, S(O), S(O)₂, or O; where each R^{h}, Rⁱ and R^{j} is independently H or lower alkyl;

when p is 1, 2, 3, 4, or 5, A₄ is N(R^{k}), C(R^{h})(Rⁱ), or O; and when p is 0, A₄ is N(R^{k})(Rⁱ), C(R^{h})(Rⁱ)(R^{j}), and O(R^{l}), where each R^{h}, Rⁱ and R^{j} is independently H or lower alkyl, each R^{k} is H, alkyl, aryl, or acyl, and each R^{l} is H, alkyl, or aryl;

provided that no more than two heteroatoms occur consecutively in the above-depicted ring formed by A₁, (A₂)ₘ, (A₃)ₚ, A₄, and C═O, where each dotted line in the ring depicts a single bond when A₂ is present and a hydrogen atom when A₂ is absent;

R^{d} is H, halogen, hydroxyl or an alkyl, alkoxy or alkylthio group, where the alkyl, alkoxy or alkylthio group is unsubstituted or substituted with one or more suitable substituents;

R^{b} is H or an alkyl group, unsubstituted or substituted with one or more suitable substituents;

R^{e} is H, halogen, hydroxyl or an alkyl, alkoxy or alkylthio group, where the alkyl, alkoxy or alkylthio group is unsubstituted or substituted with one or more suitable substituents;

Z and Z¹ are each independently H, F, an alkyl, cycloalkyl, heterocycloalkyl, aryl or heteroaryl group, where the alkyl, cycloalkyl, heterocycloalkyl, aryl or heteroaryl group is unsubstituted or substituted with one or more suitable substituents, —C(O)Rⁿ —CO₂Rⁿ —CN, —C(O)NRⁿR^{o}, —C(O)NRⁿOR^{o}, —C(S)Rⁿ, —C(S)ORⁿ —C(S)NRⁿR^{o}, —C(═NRⁿ)R^{o}, —C(═NRⁿ)OR^{o}, —NO₂, —SOR^{o}, —SO₂Rⁿ, —SO₂NRⁿR^{o}, —SO₂(NRⁿ)(OR^{o}), —SONRⁿ, —SO₃Rⁿ, —PO(ORⁿ)₂, —PO(ORⁿ)(OR^{o}), —PO(NRⁿR^{o})(OR^{p}). —PO(NRⁿR^{o})(NR^{p}R^{q}), —C(O)NRⁿNR^{o}R^{p}, —C(S)NRⁿNR^{o}R^{p}, where Rⁿ, R^{o}, R^{p} and R^{q} are each independently H or an alkyl, cycloalkyl, aryl, heterocycloalkyl, acyl or thioacyl group, where the alkyl, cycloalkyl, aryl, heterocycloalkyl, acyl or thioacyl group is unsubstituted or substituted with one or more suitable substituents, or where any two of the Rⁿ, R^{o}, R^{p} and R^{q} taken together with the atoms to which they are bonded, form a heterocycloalkyl group, which may be optionally substituted,

or Z and R^{d}, together with the atoms to which they are bonded, form a cycloalkyl or heterocycloalkyl group, where Z and R^{d} are as defined above except for moieties that cannot form the cycloalkyl or heterocycloalkyl group,

or Z and Z¹, together with the atoms to which they are bonded, form a cycloalkyl or heterocycloalkyl group, where Z and Z¹ are as defined above (except for moieties that cannot form the cycloalkyl or heterocycloalkyl group);

or a prodrug, pharmaceutically acceptable salt, pharmaceutically active metabolite, or pharmaceutically acceptable solvate thereof.

In another aspect, the invention provides a method of interfering with or preventing SARS related coronavirus viral replication activity comprising contacting a SARS related coronavirus protease with a therapeutically effective amount of a rhinovirus 3C protease inhibitor of formula IIIC:

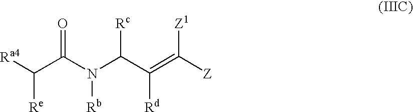

wherein:

R^{a4} is an aryloxy, heteroaryloxy, alkyloxy, cycloalkyloxy, heterocycloalkyloxy, aryl, cycloalkyl, or heteroaryl group, where the aryloxy, heteroaryloxy, alkyloxy, cycloalkyloxy, heterocycloalkyloxy, aryl, cycloalkyl, or heteroaryl group is unsubstituted or substituted with one or more suitable substituents; and

R^{c} is a substituent having the formula:

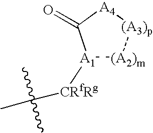

wherein:

R^{f} and R^{g} are each independently H or lower alkyl;

m is 0 or 1;

p is an integer of from 0 to 5;

A₁ is CH or N;

when p is 1, 2, 3, 4, or 5, A₂ is C(R^{h})(Rⁱ), N(R^{j}), S, S(O), S(O)₂, or O, and when p is 0, A₂ is C(R^{h})(Rⁱ)(R^{j}), N(Rⁱ)(R^{j}), S(Rⁱ), S(O)(Rⁱ), S(O)₂(Rⁱ), or O(Rⁱ), where each R^{h}, Rⁱ and R^{j} is independently H or a lower alkyl group;

each A₃ present is independently C(R^{h})(Rⁱ), N(R^{j}), S, S(O), S(O)₂, or O; where each R^{h}, Rⁱ and R^{j} is independently H or lower alkyl;

when p is 1, 2, 3, 4, or 5, A₄ is N(R^{k}), C(R^{h})(Rⁱ), or O; and when p is 0, A₄ is N(R^{k})(Rⁱ), C(R^{h})(Rⁱ)(R^{j}), and O(Rⁱ), where each R^{h}, Rⁱ and R^{j} is independently H or lower alkyl, each R^{k} is H, alkyl, aryl, or acyl, and each R^{l} is H, alkyl, or aryl;

provided that no more than two heteroatoms occur consecutively in the above-depicted ring formed by A₁, (A₂)ₘ, (A₃)ₚ, A₄, and C═O, where each dotted line in the ring depicts a single bond when A₂ is present and a hydrogen atom when A₂ is absent;

R^{d} is H, halogen, hydroxyl or an alkyl, alkoxy or alkylthio group, where the alkyl, alkoxy or alkylthio group is unsubstituted or substituted with one or more suitable substituents;

R^{b} is H or an alkyl group, unsubstituted or substituted with one or more suitable substituents;

R^{e} is H, halogen, hydroxyl or an alkyl, alkoxy or alkylthio group, where the alkyl, alkoxy or alkylthio group is unsubstituted or substituted with one or more suitable substituents;

Z and Z¹ are each independently H, F, an alkyl, cycloalkyl, heterocycloalkyl, aryl or heteroaryl group, where the alkyl, cycloalkyl, heterocycloalkyl, aryl or heteroaryl group is unsubstituted or substituted with one or more suitable substituents, —C(O)Rⁿ —CO₂Rⁿ —CN, —C(O)NRⁿR^{o}, —C(O)NRⁿOR^{o}, —C(S)Rⁿ, —C(S)ORⁿ —C(S)NRⁿR^{o}, —C(═NRⁿ)R^{o}, —C(═NRⁿ)OR^{o}, —NO₂, —SOR^{o}, —SO₂Rⁿ, —SO₂NRⁿR^{o}, —SO₂(NRⁿ)(OR^{o}), —SONRⁿ, —SO₃Rⁿ, —PO(ORⁿ)₂, —PO(ORⁿ)(OR^{o}), —PO(NRⁿR^{o})(OR^{p}). —PO(NRⁿR^{o})(NR^{p}R^{q}), —C(O)NRⁿNR^{o}R^{p}, —C(S)NRⁿNR^{o}R^{p}, where Rⁿ, R^{o}, R^{p} and R^{q} are each independently H or an alkyl, cycloalkyl, aryl, heterocycloalkyl, acyl or thioacyl group, where the alkyl, cycloalkyl, aryl, heterocycloalkyl, acyl or thioacyl group is unsubstituted or substituted with one or more suitable substituents, or where any two of the Rⁿ, R^{o}, R^{p} and R^{q}, taken together with the atoms to which they are bonded, form a heterocycloalkyl group, which may be optionally substituted,

or Z and R^{d}, together with the atoms to which they are bonded, form a cycloalkyl or heterocycloalkyl group, where Z and R^{d} are as defined above except for moieties that cannot form the cycloalkyl or heterocycloalkyl group,

or Z and Z¹, together with the atoms to which they are bonded, form a cycloalkyl or heterocycloalkyl group, where Z and Z¹ are as defined above (except for moieties that cannot form the cycloalkyl or heterocycloalkyl group);

or a prodrug, pharmaceutically acceptable salt, pharmaceutically active metabolite, or pharmaceutically acceptable solvate thereof.

In another aspect, the invention provides a method of interfering with or preventing SARS related coronavirus viral replication activity comprising contacting a SARS related coronavirus protease with a therapeutically effective amount of a rhinovirus 3C protease inhibitor of formula IV:

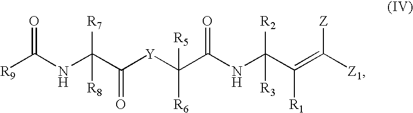

wherein:

Y is —N(R_{y})—, —C(R_{y})(R_{y})—, or —O—, where each R_{y} is independently H or lower alkyl;

R₁ is H, F, an alkyl group, OH, SH, or an O-alkyl group;

R₂ and R₃ are each independently H;

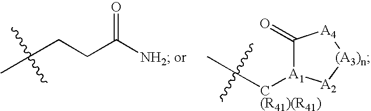

where n is an integer from 0 to 5, A₁ is CH or N, A₂ and each A₃ are independently selected from C(R₄₁)(R₄₁), N(R₄₁), S, S(O), S(O)₂, and O, and A₄ is NH or NR₄₁, where each R₄₁ is independently H or lower alkyl, provided that no more than 2 heteroatoms occur consecutively in the ring formed by A₁, A₂, (A₃)ₙ, A₄ and C═O; and provided that at least one of R₂ and R₃ is

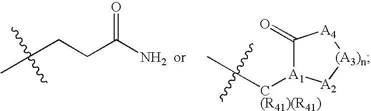

R₅ and R₆ are each independently H, F, an alkyl group, a cycloalkyl group, a heterocycloalkyl group, an aryl group, or a heteroaryl group;

R₇ and R₈ are each independently H, an alkyl group, a cycloalkyl group, a heterocycloalkyl group, an aryl group, a heteroaryl group, —OR₁₇, —SR₁₇, —NR₁₇R₁₈, —NR₁₉NR₁₇R₁₈, or —NR₁₇OR₁₈, where R₁₇, R₁₈, and R₁₉ are each independently H, an alkyl group, a cycloalkyl group, a heterocycloalkyl group, an aryl group, a heteroaryl group, or an acyl group;

R₉ is a five-membered heterocycle having from one to three heteroatoms selected from O, N, and S, or R₉ is

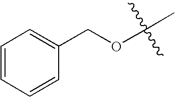

where R₂ is

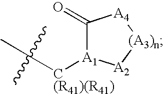

and

Z and Z₁ are each independently H, F, an alkyl group, a cycloalkyl group, a heterocycloalkyl group, an aryl group, a heteroaryl group, —C(O)R₂₁, —CO₂R₂₁, —CN, —C(O)NR₂₁R₂₂, —C(O)NR₂₁R₂₂, —C(S)R₂₁, —C(S)NR₂₁R₂₂, —NO₂, —SOR₂₁, —SO₂R₂₁, —SO₂NR₂₁R₂₂, —SO(NR₂₁)(OR₂₂), —SONR₂₁, —SO₃R₂₁, —PO(OR₂₁)₂, —PO(R₂₁)(R₂₂), —PO(NR₂₁R₂₂)(OR₂₃), PO(NR₂₁R₂₂)(NR₂₃R₂₄), —C(O)NR₂₁NR₂₂R₂₃, or —C(S)NR₂₁NR₂₂R₂₃, where R₂₁, R₂₂, R₂₃, and R₂₄ are each independently H, an alkyl group, a cycloalkyl group, a heterocycloalkyl group, an aryl group, a heteroaryl group, an acyl group, or a thioacyl group, or any two of R₂₁, R₂₂, R₂₃, and R₂₄, together with the atom(s) to which they are bonded, form a heterocycloalkyl group, provided that Z and Z₁ are not both H;

or Z₁ and R₁, together with the atoms to which they are bonded, form a cycloalkyl or heterocycloalkyl group;

or Z and Z₁, together with the atoms to which they are bonded, form a cycloalkyl or heterocycloalkyl group;

or a prodrug, pharmaceutically active metabolite, pharmaceutically acceptable salt, or solvate thereof.

In another aspect, the invention provides a method of interfering with or preventing SARS related coronavirus viral replication activity comprising contacting a SARS related coronavirus protease with a therapeutically effective amount of a rhinovirus 3C protease inhibitor of the formula V[S11]:

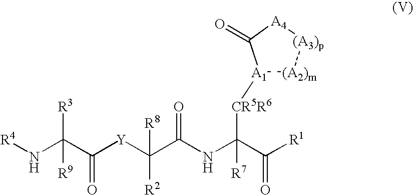

wherein:

Y is —N(R^{y})—, —C(R^{y})(R^{y})—, or —O—, where each R^{y} is independently H or lower alkyl;

R¹ is selected from optionally substituted alkyl, cycloalkyl, heterocycloalkyl, aryl, heteroaryl, and —C(O)R¹⁶, where R¹⁶ is selected from optionally substituted alkyl, cycloalkyl, heterocycloalkyl, aryl, heteroaryl, alkoxy, cycloalkoxy, heterocycloalkoxy, aryloxy, heteroaryloxy, and amine;

R² and R⁸ are each independently selected from H, F, and optionally substituted alkyl, cycloalkyl, heterocycloalkyl, aryl, and heteroaryl;

R³ and R⁹ are each independently selected from H and optionally substituted alkyl, cycloalkyl, heterocycloalkyl, aryl, heteroaryl, —OR¹⁷,—SR¹⁷, —NR¹⁷R¹⁸, —NR¹⁹NR¹⁷R¹⁸, and —NR¹⁷OR¹⁸, where R¹⁷,

R¹⁸, and R¹⁹ are each independently selected from H, alkyl, cycloalkyl, heterocycloalkyl, aryl, heteroaryl, and acyl;

R⁴is a suitable organic moiety;

each of R⁵, R⁶ and R⁷is independently H, F, or lower alkyl;

m is 0 or 1;

p is 0, 1, 2, 3, 4, or 5;

A₁ is CH or N;

when m is 1, A₂ is selected from C(R¹⁰)(R¹¹), N(R¹²), S, S(O), S(O)₂, and O;

when p is not 0, each A₃ is independently selected from C(R¹⁰)(R¹¹), N(R¹²), S, S(O), S(O)₂, and O;

where R¹⁰, R¹¹ and R¹² are each independently H or lower alkyl;

when p is not 0, A₄ is selected from N(R¹³), C(R¹⁰)(R¹¹), and O, and when p is 0, A₄ is selected from N(R¹³)(R¹⁴), C(R¹⁰)(R¹¹)(R¹²), and O(R¹⁴), provided that when A₄ is O(R¹⁴), A₁ is not CH; where R¹⁰, R¹¹ and R¹² are each independently H or lower alkyl, R¹³ is H, alkyl, aryl, or acyl, and R¹⁴ is H, alkyl, or aryl;

provided that A₁, (A₂)ₘ, (A₃)ₚ, and A₄ together do not include more than two consecutive heteroatoms;

or a prodrug, pharmaceutically acceptable salt, pharmaceutically active metabolite, or pharmaceutically acceptable solvate thereof.

In another aspect, the invention provides a method of interfering with or preventing SARS related coronavirus viral replication activity comprising contacting a SARS related coronavirus protease with a therapeutically effective amount of a rhinovirus 3C protease inhibitor of formula VI:

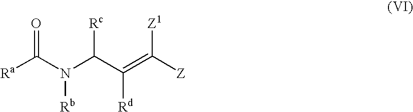

wherein:

R^{a} is an alkylcarbonylalkyl, cycloalkylcarbonylalkyl, arylcarbonylalkyl, heteroarylcarbonylalkyl, alkylcarbonylaminoalkyl, cycloalkylcarbonylaminoalkyl, heterocycloalkylcarbonylaminoalkyl, arylcarbonylaminoalkyl, heteroarylcarbonylaminoalkyl, alkylaminocarbonylalkyl, cycloalkylaminocarbonylalkyl, heterocycloalkylaminocarbonylalkyl, arylaminocarbonylalkyl, heteroarylaminocarbonylalkyl group, where each alkyl, cycloalkyl, heterocycloalkyl, aryl and heteroaryl moiety thereof may be unsubstituted or substituted with one or more suitable substituents;

R^{b} is H or an alkyl group, unsubstituted or substituted with one or more suitable substituents;

R^{d} is H, halo, hydroxyl, or an alkyl, alkoxy or alkylthio group, where the alkyl, alkoxy or alkylthio group is unsubstituted or substituted with one or more suitable substituents;

R^{c} is a moiety having the formula:

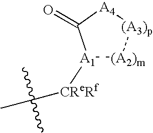

R^{e} and R^{f} are each independently H or a lower alkyl group;

m is 0 or 1, provided that when m is 1, R^{a} is not an amino-substituted alkylcarbonylalkyl or amino-substituted alkylcarbonylaminoalkyl group, and when m is 0, R^{a} is selected from an alkylaminocarbonylalkyl, cycloalkylaminocarbonylalkyl, heterocycloalkylaminocarbonylalkyl, arylaminocarbonylalkyl, heteroarylaminocarbonylalkyl and heteroarylcarbonylaminoalkyl group, provided that R^{a} is not substituted indolecarbonylaminoalkyl;

p is an integer of from 0 to 5;

A₁ is CH or N;

when p is 1, 2, 3, 4, or 5, A₂ is C(R^{g})(R^{h}), N(Rⁱ), S, S(O), S(O)₂, or O, and when p is 0, A₂ is C(R^{g})(R^{h})(Rⁱ), N(R^{g})(Rⁱ), S(R^{g}), S(O)(R^{g}), S(O)₂(R^{g}), or O(R^{g}), where each R^{g}, R^{h} and Rⁱ is independently H or a lower alkyl group;

each A₃ present is independently C(R^{g})(R^{h}), N(Rⁱ), S, S(O), S(O)₂, or O, where each R^{g}, R^{h} and Rⁱ is independently H or a lower alkyl group;

when p is 1, 2, 3, 4, or 5, A₄ is N(R^{j}), C(R^{g})(R^{h}), or O, and when p is 0, A₄ is N(R^{j})(R^{k}), C(R^{g})(R^{h})(Rⁱ), and O(R^{k}), where each R^{g}, R^{h} and Rⁱ is independently H or a lower alkyl group, each R^{j} is H, an alkyl, aryl, or acyl group, and each R^{k} is H or an alkyl or aryl group;

provided that no more than two heteroatoms occur consecutively in the above-depicted ring formed by A₁, (A₂)ₘ, (A₃)ₚ, A₄, and C═O, where each dotted line in the ring depicts a single bond when A₂ is present and a hydrogen atom when A₂ is absent; and

Z and Z¹ are each independently H, F, an alkyl, cycloalkyl, heterocycloalkyl, aryl or heteroaryl group, where the alkyl, cycloalkyl, heterocycloalkyl, aryl or heteroaryl group is unsubstituted or substituted with one or more suitable substituents, —C(O)R^{l}, —CO₂R^{l}, —CN, —C(O)NR^{l}Rₘ, —C(O)NR^{l}OR^{m}, —C(S)R^{l}, —C(S)OR^{l} —C(S)NR^{l}R^{m}, —C(═NR^{l})R^{m}, —C(═NR^{l})OR^{m}, —NO₂, —SOR^{m}, —SO₂R^{l}, —SO₂NR^{l}R^{m}, —SO₂(NR^{l})(OR^{m}), —SONR^{l}, —SO₃R^{l}, —PO(OR^{l})₂, —PO(OR^{l})(OR^{m}), —PO(NR^{l}R^{m})(ORⁿ), —PO(NR^{l}R^{m})(NRⁿR^{o}), —C(O)NR^{l}NR^{m}Rⁿ, —C(S)NR^{l}NR^{m}Rⁿ, where R^{l}, R^{m}, Rⁿ and R^{o} are each independently H or an alkyl, cycloalkyl, aryl, heterocycloalkyl, acyl or thioacyl group, where the alkyl, cycloalkyl, aryl, heterocycloalkyl, acyl or thioacyl group is unsubstituted or substituted with one or more suitable substituents, or where any two of the R^{l}, R^{m}, Rⁿ and R^{o}, taken together with the atoms to which they are bonded, form a heterocycloalkyl group, which may be optionally substituted,

or Z and R^{d},together with the atoms to which they are bonded, form a cycloalkyl or heterocycloalkyl group, where Z and R^{d} are as defined above except for moieties that cannot form the cycloalkyl or heterocycloalkyl group,

or Z and Z¹, together with the atoms to which they are bonded, form a cycloalkyl or heterocycloalkyl group, where Z and Z¹ are as defined above;

or a prodrug, pharmaceutically acceptable salt, pharmaceutically active metabolite, or pharmaceutically acceptable solvate of said compound.

In another aspect, the invention provides a method of interfering with or preventing SARS related coronavirus viral replication activity comprising contacting a SARS related coronavirus protease with a therapeutically effective amount of a rhinovirus 3C protease inhibitor of formula VII:

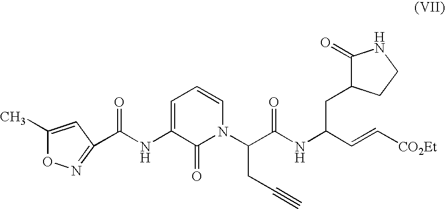

or a prodrug, pharmaceutically acceptable salt, or pharmaceutically acceptable solvate thereof.

In another aspect, the invention provides a method of interfering with or preventing SARS related coronavirus viral replication activity comprising contacting a SARS related coronavirus protease with a therapeutically effective amount of a rhinovirus 3C protease inhibitor of formula VIIA:

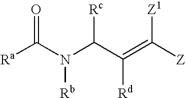

wherein:

R^{a} is substituted or unsubstituted heterocycloalkyl or heterocycloalkylalkyl;

R^{b} is a substituent having the formula:

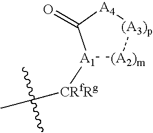

wherein:

R^{f} and R^{g} are independently H or lower alkyl;

m is 1;

p is an integer of from 1 to 5;

A₁ is CH or N;

A₂ is C(R^{h})(Rⁱ), N(R^{j}), S, S(O), S(O)₂, or O; where each R^{h}, Rⁱ and R^{j} is independently H or lower alkyl;

each A₃ present is independently C(R^{h})(Rⁱ), N(R^{j}), S, S(O), S(O)₂, or O; where each R^{h}, Rⁱ and R^{j} is independently H or lower alkyl;

A₄ is N(R^{k}), C(R^{h})(Rⁱ), or O;

provided that no more than two heteroatoms occur consecutively in the above-depicted ring formed by A₁, (A₂)ₘ, (A₃)ₚ, A₄, and C═O, where each dotted line in the ring depicts a single bond;

R^{c} is H, halogen or a substituted or unsubstituted lower alkyl group;

R^{d} is H, halogen, hydroxyl, a substituted or unsubstituted alkyl, alkoxy or alkylthio group;

R^{e} is H or a substituted or unsubstituted alkylgroup; and

Z and Z¹ are independently H, F, a unsubstituted or substituted alkyl group, cycloalkyl group, heterocycloalkyl group, aryl group or heteroaryl group, —C(O)Rⁿ, —CO₂Rⁿ, —CN, —C(O)NRⁿR^{o}, —C(O)NRⁿOR^{o}, —C(S)Rⁿ, —C(S)ORⁿ, —C(S)NRⁿR^{o}, —NO₂, —SOR^{o}, —SO₂Rⁿ, —SO₂NRⁿR^{o}, —SO₂(NRⁿ)(OR^{o}), —SONRⁿ, —SO₃Rⁿ, —PO(ORⁿ)₂, —PO(ORⁿ)(OR^{o}), —PO(NRⁿR^{o})(OR^{p}), —PO(NRⁿR^{o})(NR^{p}R^{q}), —C(O)NR NR^{o}R^{p}, or —C(S)NR NR^{o}R^{p}, wherein Rⁿ, R^{o}, R^{p} and R^{q} are independently H, a substituted or unsubstituted alkyl group, cycloalkyl group, aryl group, heterocycloalkyl group, acyl group or thioacyl group, or wherein any two of the Rⁿ, R^{o}, R^{p} and R^{q}, taken together with the atoms to which they are bonded, form a heterocycloalkyl group, which may be optionally substituted,

or Z and R^{d}, together with the atoms to which they are bonded, form a cycloalkyl or heterocycloalkyl group,

or Z and Z¹, together with the atom to which they are bonded, form a cycloalkyl or heterocycloalkyl group;

or a prodrug, pharmaceutically acceptable salt, or pharmaceutically acceptable solvate thereof.

In another aspect, the invention provides a method of interfering with or preventing SARS related coronavirus viral replication activity comprising contacting a SARS related coronavirus protease with a therapeutically effective amount of a rhinovirus 3C protease inhibitor selected from the group consisting of:

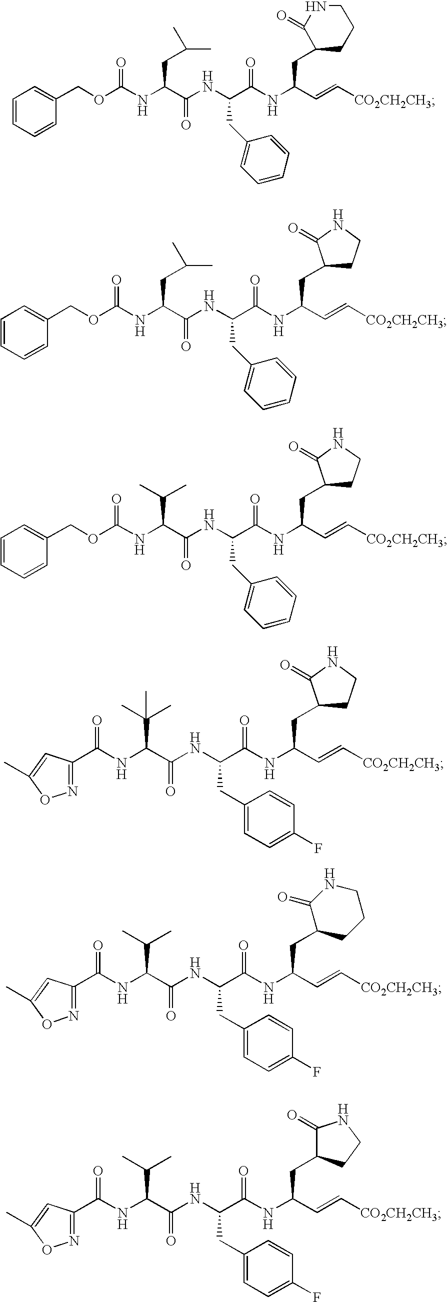

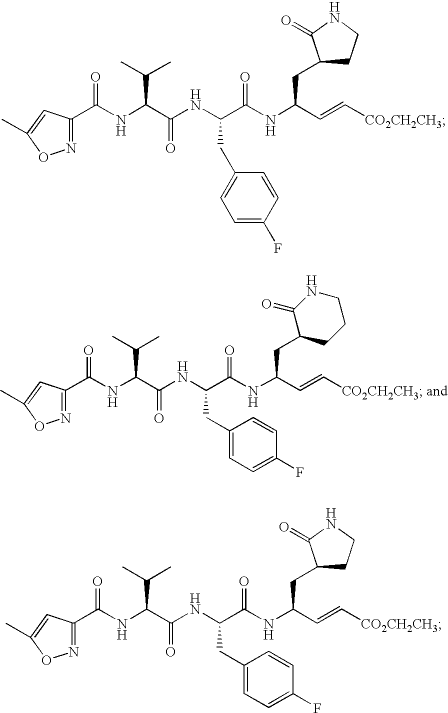

and the pharmaceutically acceptable salt, pharmaceutically active metabolite, or solvate thereof.

In still another embodiment of the present invention, the method described above utilizes the rhinovirus inhibitor selected from the group consisting of:

2-(2-Phenylethyl)benzoic acid N-hydroxyamide;

2-(Propylthio)-pyridine-3-N-(hydroxy)carboxamide;

[4-(N-Hydroxyamino)-2R-isobutyl-3S-((thien-2-yllthio)methyl)succinyl]-L-phenylalanine-N-methylamide;

N-Hydroxy-5-phenylpentanamide;

2-(Phenyl-2-ethyl)pyridine-3-N-hydroxycarboxamide;

2-(Thiobenzyl)benzoic acid N-hydroxy amide;

6-Biphenyl-4-yl-[2,2-dimethyl-1-(pyridin-4-ylcarbamoyl)-propylcarbamoyl]-hexanoic acid, N-hydroxyamide;

3R(6-(4-Biphenyl)-3-(N-benzylcarbamoyl))-hexanoic acid N-hydroxyamide;

2-Benzylsulfonyl-cyclopent-1-ene-carboxylic acid hydroxamide;

2-Benzylsulfonyl-cyclohex-1-enecarboxylic acid hydroxyamide;

6-Benzylsulfonyl-cyclohex-1-enecarboxylic acid hydroxyamide;

1-(N-Hydroxy)-3-(2-bibenzyl)urea;

3R-(6-(4-Biphenyl)propyl)-N-(3-methylpyridinecarbamoyl)-hexanoic acid N-hydroxy-amide;

4-(2-{[5-Hydroxyamino-3-(3-phenyl-propyl)-3,4-dihydro-2-H-pyrrole-3-carbonyl]-amino}-4-methyl-pentanoylamino)benzoic acid methyl ester;

5-Hydroxyamino-3-(3-phenyl-propyl)-3,4-dihydro-2-H-pyrrole-3-carboxylic acid (2-cyclohexyl-1-methylcarbamoyl-ethyl) amide;

4-(2-{[5-Hydroxyamino-3-(3-pentyl)-3,4-dihydro-2-H-pyrrole-3-carbonyl]-amino}-4-methyl-pentanoylamino) benzoic acid methyl ester;

6-Biphenyl-4-yl-3-(R)-(2-hydroxy-1-hydroxymethyl-ethylcarbamoyl)-hexanehydroxamic acid;

6-Biphenyl-4-yl-3(R)-(1(S)-hyroxymethyl-2,2-dimethyl-propylcarbamoyl)-hexanehydroxamicacid;

2-(Biphenyl-4-ylsulfonyl)-cyclohex-1-enecarboxylic acid hydroxyamide;

6-(Biphenyl-4-ylsulfonyl)-cyclohex-1-enecarboxylic acid hydroxyamide;

2-Phenethylsulfanyl-cyclohex-I-enecarboxylic acid hydroxyamide;

2-Benzylsulfanyl-cyclohexancarboxylic acid hydroxamide;

trans-2-Benzylsulfanyl-cyclohexancarboxylic acid hydroxamide;

trans-2-(Biphenyl-4-yl-methylsulfanyl)-cyclohexancarboxylic acid hydroxamide;

6-Biphenyl-4-yl-3-(R)-(1-hydroxymethyl-2-(S)-(1H-imidazol-4-yl)-ethylcarbamoyl)-hexanehydroxamic acid;

N-Hydroxy-2-[2-Oxo-3-(3-phenyl-propyl)-tetrahydro-furan-3-yl]-acetamide;

trans-2-(4-Phenoxy-benzylsulfanyl)-cyclohexancarboxylic acid hydroxamide;

2-(4-Indol-1-yl-benzylsulfanyl)-cyclohexancarboxylic acid hydroxamide;

2-(3-Biphenyl-4-yl-propyl)-N4-hydroxy-N1-(2,4,5-trihydroxy-6-hydroxymethyl-tetrahydro-pyran-3-yl)-succinamide;

2-(2-Biphenyl-4-yl-ethylsulfanyl)-cyclohexane carboxylic acid hydroxyamide;

2-(3-Biphenyl-4-yl-propyl)-N4-hydroxy-N 1-(2-hydroxy-cyclohexyl)-succinamide;

6-Biphenyl-4-yl-3-(1-hydroxyimino-ethyl)-hexanoic acid hydroxyamide;

3-(R)-(2-Hydroxy-1-(S)-(1H-imidazol-4-yl)-ethylcarbamoyl)-6-(4-(2-methyl-thiazol-4-yl)-phenyl)-hexanehydroxamic acid;

6-Biphenyl-4-yl-3-(3-hydroxy-piperidine-1-carbonyl)-hexanoic acid-hydroxyamide;

1-(4-Methoxy-benzenesulfonyl)-piperidine-2-carboxylic acid hydroxamide;

1-1-[4-Bromo-phenoxy)-benzenesulfonyl)-piperidine-2-carboxylic acid hydroxyamide;

N-(1-benzyl-2-hydroxy-ethyl)-N4-hydroxy-2-isobutyl-succinamide;

6-Biphenyl-4-yl-3(R)-2(S)-hydroxy-(1(S)-hydroxymethyl-2,2-dimethyl-propylcarbamoyl)-hexanoic hydroxamic acid;

6-Biphenyl-4-yl-3-(2-hydroxy-1-hydroxmethyl-propylcarbamoyl)-hexanoic hydroxamic acid;

trans-2-(3-Biphenyl-4-yl-propyl)-cyclohexane carboxylic acid hydroxyamide;

1-[4-Biphenyl-4-yloxy)-benzenesulfonyl)-piperidine-2-carboxylic acid hydroxamide;

1-(4-Phenoxy-benzenesulfonyl)-piperidine-2-carboxylic acid hydroxamide;

6-Biphenyl-4-yl-3-(R)-(1-(S)-hydroxymethyl-2-(3-pyridyl)-ethylcarbamoyl)-hexanehydroxamic acid;

6-Biphenyl-4-yl-2S-hydroxy-3R-(1S-hydroxymethyl-3-methylsulfanyl-propylcarbamoyl)-hexanoic hydroxamic acid;

1-[-[4-(4-Bromo-phenoxy)-benzenesulfonyl]-4-(tertbutoxyarbonyl)-piperazine-2-carboxylic acid hydroxyamide;

1-[4-(4-Bromo-phenoxy)-benzenesulfonyl]-piperazine-2-carboxylic acid hydroxyamide;

4-Acetyl-1-[4-phenoxy-benzenesulfonyl]-piperazine-2-carboxylic acid, N-hydroxyamide;

1-(Diphenylphosphinic)-piperidine-2-carboxylic acid hydroxamide;

6-Biphenyl-4-yl-3-(R)-(2-oxo-1-tetrahydrofuran-3-(S)-ylcarbamoyl)-hexane hydroxamic acid;

1-[-[4-(4-Bromo-phenoxy)-benzenesulfonyl]-4-methyl-piperazine-2-carboxylic acid N-hydroxyamide;

4-(4-Methoxy-benzenesulfonyl)-thiomorpholine-3-carboxylic acid hydroxyamide;

3-(Diphenylphosphinic)-propanoic acid hydroxyamide;

1-[4-(4-Chlorophenoxy)benzenesulfonyl]-thiomorpholine-3-carbamoyl)piperazine-2-carboxamide;

4[4-Phenoxy-benzenesulfonyl]-piperazine-2-carboxylic acid, N-hydroxyamide;

4[4-Phenoxy-benzenesulfonyl]-thiomorpholine-3-carboxylic acid N-hydroxyamide;

3[2-Biphenyl-4-yl-ethylsulfanyl]-tetrahydro-pyran-4-carboxylic acid N-hydroxyamide;

1-[4-Phenoxy-benzenesulfonyl]-4-methyl-piperazine-2-carboxylic acid N-hydroxyamide;

6-Biphenyl-4-yl-3-(R)-(2-oxo-azepan-3-(S)-ylcarbamoyl)-hexane hydroxamic acid;

4-(1H-Indole-2-sulfonyl)-thiomorpholine-3-carboxylic acid hydroxyamide;

1-(Methyl-phenylphosphinic)-piperidine-2-(R)-carboxylic acid hydroxamide;

1-(1,3-Dihydro-isoindole-2-sulfonyl)-piperidine-2-carboxylic acid hydroxamide;

4-Methyl-1-(4-(4-chlorophenyl)benzenesulfonyl)-N-hydroxy-2R-piperazinecarboxamide hydrochloride;

1-[4-Chlorophenoxybenzenesulfonyl]-N-hydroxy-2R-piperazinecarboxamide;

2-(3-Phenyl-propylsulfonyl)-cyclohexane carboxylic acid hydroxamide;

1-(Pyrolidine-1-sulfonyl)-piperidine-2-carboxylic acid hydroxyamide;

1-(Piperidine-1-sulfonyl)-piperidine-2-carboxylic acid hydroxyamide;

4-[-[4-Bromo-phenoxy-benzenesulfonyl]-oxothiomorpholine-3-carboxylic acid-N-hydroxyamide;

1-[4-(4-Methoxy-phenylsulfanyl)-benzenesulfonyl]-piperdine-2-carboxylic acid hydroxyamide;

1-[4-(4-Cyano-phenoxy)-benzenesulfonyl]-4-(tert-butoxycarbonyl)-piperazine-2-carboxylic acid N-hydroxyamide;

6-Oxo-3-(4-phenoxy-benzenesulfonyl)-hexahydro-pyrimidine-4-carboxylic acid hydroxamate;

4-(t-Butoxycabonyl)-1-(4-(pyridin-2-yl)oxybenzensulfonyl)-N-hydroxy-piperazine-2-carboxamide;

4-[(4-Fluorophenoxy)-benzenesulfonyl]-thiomorpholine-3-carboxylic acid N-hydroxyamide;

4-[4-(Fluoro-phenoxy)-benzenesulfonyl]-oxothiomorpholine-3-carboxytic acid N-hydroxyamide;

4-(4-Butoxy-benzenesulfonyl)-thiomorpholine-3-carboxylic acid hydroxyamide;

4-(4-Butoxy-benzenesulfonyl)-1-oxothiomorpholine-3-carboxylic acid hydroxyamide;

1-[4-(4-Fluorophenyl)benzenesulfonyl]-4-(tert-butoxycarboxyl)2R-piperazine-2-carboxylic acid hydroxyamide;

1-((4-(4-Chlorophenyl)-piperazine)-1-sulfonyl)-piperidine-2carboxylic acid hydroxamide;

cis-2-Phenethylsulfanyl-cyclohexanecarboxylic acid hydroxyamide;

1-[-[4-(4-Fluorophenyl) benzenesulfonyl)-N-hydroxy-2R-piperazinecarboxamide hydrochoride;

1-(Diphenylphosphinic)-pyrolidine-2(R)-carboxylic acid hydroxyamide;

trans-2-Phenethylsulfonyl-cyclohexanecarboxylic acid hydroxyamide;

1-[4-(4-Flourophenyl)-piperazine-1-sulfonyl]-piperidine-2-carboxylic acid hydroxamide;

1-1-[4-(4-Fluorophenylsulfanyl)-benzenesulfonyl]-piperidine-2-carboxylic acid hydroxyamide;

4-1-[4-(Bromo-phenoxy)-benzenesulfonyl]-2,2-dimethyl-1-oxo-thiomorpholine-3-carboxylic acid hydroxyamide;

1-(Pyrrolidine-1-carbonyl)-pyrrolidine-2(R)-carboxylic acid hydroxyamide;

R-4-[4-(Bromophenoxy)-benzenesulfonyl]-2,2-dimethyl-1-oxo-thiomorpholine-3-carboxylic acid hydroxyamide;

4-(Ethoxycarbonyl)methyl-1-(4-(4-chlorophenyl)benzenesulfonyl)-N-hydroxy-2R-piperazinecarboxamide hydrochloride;

1-Phenethylcarbamoyl-pyrrolidine-2-(R)-carboxylic acid hydroxyamide;

1-(4-Benzyl-piperazine-1-sulfonyl)-piperidine-2-carboxylic acid hydroxyamide;

3(S)-N-Hydroxy-4-(4-(pyridin-4-yl) oxybenzenesulfonyl)-2,2-dimethyl-tetrahydro-2H-1,4-thiazine-3-carboxamide;

2(R)4-Methyl-1-(4-(4-fluorophenyl)benzenesulfonyl)-N-hydroxy-piperazine-2-carboxamide;

1-((2-Pyridyl)-4-piperazine-1-sulfonyl)-piperdine-2-carboxylic acid hydroxyamide;

1-1-[4-(Pyridin-4-ylsulfamyl)-benzenesulfonyl]-piperdine-2-carboxylic acid hydroxyamide;

N-(4-Phenoxy-benzenesulfonyl)-D-tert-leucine-N-hydroxyamide;

2,2-Dimethyl-4-[4-(pyridin-2-yloxy)-benzenesulfonyl]-thiomorpholine-3-carboxylic acid hydroxyamide;

N-1-[4-(4-Fluorophenoxyl) benzenesulfonyl)-D-tert-leucine, N-hydroxyamide;

3(R)-N-Hydroxy4-(4-(pyridin-4-yl) oxybenzenesulfonyl)-2,2-dimethyl-tetrahydro-2H-1,4-thiazine-3-carboxamide hydrochloride;

2-[4-(4-Chloro-phenoxy)-benzenesulfonylamino]-N-hydroxy-3,3-dimethyl-butyramide;

3(R)-N-Hydroxy-4-(4-(fur-3-yl) phenoxybenzenesulfonyl)-2, 2-dimethyl-tetrahydro-2H-1,4-thiazine-3-carboxamide;

2-1-[4-(Pyridin-2-yl-oxy)-benzenesulfonylamino]-N-hydroxy-3,3-dimethyl butyramide;

2-(2-Biphenyl-4-yl-ethylsulfonyl)-cyclohex-1-ene-carboxylic acid hydroxyamide;

6-(2-Biphenyl-4-yl-ethyl sulfonyl)-cyclohex-1-ene-carboxylic acid hydroxyamide;

N-(4-Phenoxy-benzenesulfonyl)-3,3-dimethyl-S-(methylthio)-D-cysteine, N-hydroxyamide;

1-(4-Phenoxy-piperidine-1-sulfonyl)-piperdine-2-carboxylic acid hydroxyamide;

N-(4-[4-Chlorophenoxy]-benzenesulfonyl)-3,3-dimethyl-S-(methylthio)-D-cysteine, N-hydroxyamide;

N-(4-[4-Chlorophenoxy]-benzenesulfonyl)-3,3-dimethyl-S-(methylsulfoxy)-D-cysteine, N-hydroxyamide;

cis-2-(2-Phenyl-ethanesulfonyl)-cyclohexanecarboxylic acid hydroxyamide;

3(R)-N-Hydroxy-4-(4-(imidazol-1-yl) phenoxybenzenesulfonyl)-2,2-dimethyl-tetrahydro-2H-1,4-thiazine-3-carboxamide;

3(R)-N-Hydroxy-4-(4-(pyridin-4-yl) oxybenzenesulfonyl)-2,2-dimethyl-tetrahydro-2H-1,4-thiazine-3-carboxamide;

4-1-[2-(2-Hydroxycarbamylmethyl-5-pheny-pentanoylamino)-4-methyl-pentanoyl]-benzoic acid methyl ester;

trans-2-(2-Phenyl-ethanesulfonyl)-cyclohexanecarboxylic acid hydroxyamide;

3,3-Dimethyl-2-(4-phenoxy-phenylsulfanylmethyl)-butyric acid, N-hydroxyamide;

2-(2-Biphenyl-4-yl-ethanesulfonyl)-cyclohexanecarboxylic acid hydroxamate;

2-[-[4-(4-Chlorophenyl)-piperazine-1-sulfonylamino]-3-methyl-3-(pyridin-2ylmethylsulfanyl)-butyric acid N-hydroxyamide;

3,3-Dimethyl-2-(4-phenoxy-phenylsulfonylmethyl)-butyric acid, N-hydroxyamide;

2(R)-[4-(4-Fluoro-phenoxy) benzenesulfonylamino]-3-methyl-3-(pyridin-2-yl sulfanyl)-butyric acid, hydroxyamide;

3(R)-N-Hydroxy-4-(4-(-((pyridin-4-yl) methyl) oxybenzenesulfonyl)-2,2-dimethyl-tetrahydro-2H-1,4-thiazine-3-carboxamide;

1-1-[4-(4-Chloro-phenoxy)-benzenesulfonyl]-4-(1-methyl-H-imidazole-4-sulfonyl)-piperazine-2-carboxylic acid hydroxamide;

1-[4-(Pyridin-2-ylsulfanyl)-piperidine-1-sulfonyl]-piperidine-2-carboxylic acid hydroxyamide;

2R-[4-(4-Furan-3-yl-phenoxy)-benzenesulfonylamino]-N-hydroxy-3-methyl-3-(pyridin-4-ylsulfanyl)-butyramide;

trans-2-(2-Biphenyl-4-yl-ethylsu lfanyl)-cyclohexanecarboxylic acid hydroxyamide;

N4-(2,2-Dimethyl-1S-hydroxymethyl-propyl)-N1-hydroxy-3R [3-(4-pyridin-4-yl-phenyl)-pyrrol-1-yl]-succindiamide;

1-[4-(4-Fluoro-phenoxy)-benzenesulfonyl)]-3,3-dimethyl-5-oxo-piperazine-2-carboxylic acid hydroxyamide;

2(R)-[4-(4-lodo-phenoxy)benzenesulfonylamino]-3-methyl-(pyridin-3-yl-sulfonyl) butyric acid hydroxyamide;

1-[-[2-(Benzothiazol-2-ylsulfanyl)-piperidine-1-sulfonyl]piperidine-2-carboxylic acid hydroxyamide;

5-[4-(4-Fluoro-phenoxy)-benzenesulfonyl]-4, 5, 6, 7-tetrahydro-3H-imidazolo[4,5,-c]pyridine-6-carboxylic acid hydroxyamide;

1-[4-(Pyridin-4-ylsulfanyl)-piperidine-1-sulfanyl]-piperidine-2carboxylic acid hydroxyamide;

1-[4-(4-Methoxy-phenylsulfamyl)-piperidine-1-sulfonyl]piperidine-2-carboxylic acid hydroxyamide;

2(R)-[4-(4-Methylphenoxy)benzenesulfonylamino]-3-methyl-3-(pyrdin-3-yl-sulfonyl) butyric acid hydroxyamide;

1-[4-(4-Methyl-phenylsulfamyl)-piperidine-1-sulfonyl]-piperidine-2-carboxylic acid hydroxamide;

4-Methoxy-benzenesulfonyl)-2,2-dimethyl-thiomorpholine-3-carboxylic acid hydroxyamide;

4-1-[4-(4-Chloro-phenoxy)-benzenesulfonyl]-2,2-dimethyl-thiomorpholine-3-carboxylic acid hydroxyamide;

2(R)-[4-(4-bromo-phenoxy) benzenesulfoxylamino]-3-methyl-3-(pyridin-4-yl-sulfoxide) butyric acid hydroxyamide;

4-(4-Methoxy-benzensulfonyl)-2,2-dimethyl-1-oxo-thiomorpholine-3-carboxylic acid hydroxyamide;

4-4-(4-Chloro-phenoxy)-benzenesulfonyl]-2,2-dimethoxy-1-oxo-thiomorpholine-3-carboxylic acid hydroxyamide;

3(S)-2,2-Dimethyl-4-[4-(pyridin-4-ylsulfanyl)-benzenesulfonyl]-thiomorpholine-3-carboxylic acid hydroxyamide;

3,3-Dimethyl-N-hydroxy-2R-[-[4(-(pyridin-4-ylsulfanyl)-piperidine-1-sulfonylamino]-butyramide;

N-Hydroxy-2-[-[(4-methylbenzenesulfonyl) amino]acetamide;

[4(-(4-Imidazol-1-yl-phenoxy)-piperidine-1-sulfonyl]-piperidine-2-carboxylic acid hydroxyamide;

1-[4-(4-Imidazol-1-yl-phenylsulfanyl)-piperidine-1-sulfonyl]-piperidine-2-carboxylic acid hydroxyamide;

2(R)-[4-(4-Chloro-benzoyl)-cyclohexanesulfonyl]-piperidine-1-carboxylic acid hydroxyamide;

1(R)-[4-(4-Chloro-benzoyl)-piperidine-1-sulfonyl]-piperidine-2-carboxylic acid hydroxyamide;

1(R)-(4-Pyrdin-2-yl-piperazine-1-sulfonyl)-piperidine-2-carboxylic acid hydroxyamide;

1(R)-[4-(4-Imidazol-1-yl-phenoxy)-piperidine-1-sulfonyl]-piperidine-2-carboxylic acid hydroxyamide;

N-Hydroxy-3,3-dimethyl-2R-[4(-(morpholine-4-carbonyl)-piperidine-1-sulfonylamino]-butyramide;

N-Hydroxy-3-methyl-3-(5-methyl-isoxazol-3-yl-methylsulfanyl)-2R-[4-(pyridin-4-ylsulfanyl)-piperidine-sulfonylamino]-butyramide;

N-Hydroxy-2R-[4-(4-imidazol-1-yl-phenoxy)-piperidine-1-sulfonylamino]-3,3-dimethyl-butyramide;

2R-[4-(4-Chloro-benzoyl)-piperazine-1-sulfonylamino]-Nhydroxy-3-methyl-3-methylsulfanyl-butyramide;

N-Hydroxy-3-methyl-3-methylsulfanyl-2R-[4-(pydin-4--ylsonylamino]-butyramide;

1R,3S,2,2-Dimethyl-1-oxo-4-[-[4(-(pyridin-4-yloxy)-benzenesulfonyl]-thiomorpholine-3-carboxylicacid amide;

4-(2-(4-fluorobenzyl)-6-methyl-5-[(5-methylisoxazole-3-carbonyl)-amino]-4-oxoheptanoylamino-5-(2-oxopyrrolidin-3-yl)-pent-2-enoic acid ethyl ester; and the pharmaceutically acceptable salts thereof.

Other embodiments of the invention include those described in U.S. application Ser. Nos. 08/825331, 08/991282 (U.S. Pat. No. 6,020,371), 09/421560 (U.S. Pat. No. 6,331,554), 09/947381 (Pub. No. 0032237A-1), 09/567148 (U.S. Pat. No. 6,369,226), 09/882345 (Pub. No. 0061916), 09/301977 (U.S. Pat. No. 6,531,452), 09/647712, 10/289982, 09/631708 (U.S. Pat. No. 6,534,530), 09/834783 (Pub. No. 0006943-Al), and 09/726376 (U.S. Pat. No. 6,514,997), which are incorporated herein in their entirety by reference.

### BRIEF DESCRIPTION OF THE FIGURES

FIG. 1 is a sequence alignment of 3C-like protein translated from SARS genome (AY274119) with TGEV 3C-like proteinase (1LVO) used for homology modeling. The location of the first indel was adjusted from the BLAST alignment to better reflect the multiple alignment of other coronavirus 3C-like proteins (Anand, Palm et al. 2002). 43% of the residues are identical in this alignment.

FIG. 2 depicts the twelve residues used to superimpose the 3C-like protein structures were identified by visual inspection. They include a region near the catalytic cysteine, the catalytic histidine, and a region of structurally conserved beta-strand.

FIG. 3 is a homology model for SARS 3C-like protease (atom-color wire) superimposed on the cocrystal structure of rhinovirus 3C protease (purple wire) bound to AG7088 (atom-color stick).

FIG. 4 shows the hydrogen bond between AG7088 and rhinovirus 3C protease from the cocrystal structure (1CQQ), the corresponding hydrogen bonds between AG7088 and the model of SARS 3C protease when superimposed on the structure of rhinovirus 3C protease. Four of the hydrogen bonds predicted between AG7088 and the SARS 3C protease model are also found in the cocrystal structure of TGEV (1LVO), where water or the small molecule 2-methyl-2,4-pentanediol replace the inhibitor.

FIG. 5 shows solvent accessible (Connolly) surface of the binding site of AG7088 in the crystal structure of rhinovirus 3C protease (upper panel) and the corresponding surface in the SARS 3C protease model (lower panel).

FIG. 6 shows the percent (%) identity between coronavirus 3C proteases including SARS (AY274119), MHV: murine hepatitis virus (M55148), BCoV: bovine coronavirus (Q8V440), PEDV: porcine epidemic diarrhea virus (Q91AV2), FIPV: feline infectious peritonitis virus (Q98VG9), TGEV: transmissible gastroenteritis virus (Q91W05), HCoV: human coronavirus 229E (Q9DLN0), AIBV: avian infectious bronchitis virus (M95169).

FIG. 7 is a phylogenetic tree describing the coronavirus 3C proteases.

FIG. 8 is a molecular model of compound 1 in the binding site of SARS 3C like protease.

FIG. 9 is a molecular model of compound 2 in the binding site of SARS 3C like protease.

FIG. 10 is a molecular model of compound 3 in the binding site of SARS 3C like protease.

FIG. 11 is a molecular model of compound 4 in the binding site of SARS 3C like protease.

FIG. 12 is a molecular model of compound 5 in the binding site of SARS 3C like protease.

FIG. 13 is a molecular model of compound 6 in the binding site of SARS 3C like protease.

FIG. 14 is a molecular model of compound 7 in the binding site of SARS 3C like protease.

FIG. 15 is a molecular model of compound 8 in the binding site of SARS 3C like protease.

FIG. 16 is a molecular model of compound 9 in the binding site of SARS 3C like protease.

FIG. 17 is a molecular model of compound 10 in the binding site of SARS 3C like protease.

FIG. 18 is a molecular model of compound 11 in the binding site of SARS 3C like protease.

FIG. 19 is a molecular model of compound 12 in the binding site of SARS 3C like protease.

FIG. 20 is a molecular model of compound 13 in the binding site of SARS 3C like protease.

FIG. 21 is a molecular model of compound 14 in the binding site of SARS 3C like protease.

FIG. 22 is a molecular model of compound 15 in the binding site of SARS 3C like protease.

FIG. 23 is a molecular model of compound 16 in the binding site of SARS 3C like protease.

FIG. 24 is a molecular model of compound 17 in the binding site of SARS 3C like protease.

FIG. 25 is a molecular model of compound 18 in the binding site of SARS 3C like protease.

FIG. 26 is a molecular model of compound 19 in the binding site of SARS 3C like protease.

FIG. 27 is a molecular model of compound 20 in the binding site of SARS 3C like protease.

FIG. 28 is a molecular model of compound 21 in the binding site of SARS 3C like protease.

FIG. 29 is a molecular model of compound 22 in the binding site of SARS 3C like protease.

FIG. 30 is a molecular model of compound 23 in the binding site of SARS 3C like protease.

FIG. 31 is a molecular model of compound 24 in the binding site of SARS 3C like protease.

FIG. 32 is a molecular model of compound 25 in the binding site of SARS 3C like protease.

FIG. 33 is a molecular model of compound 26 in the binding site of SARS 3C like protease.

### DETAILED DESCRIPTION OF THE INVENTION AND PREFERRED EMBODIMENTS

For purposes of the present invention, as described and claimed herein, the following terms are defined as follows:

As used herein, the terms “comprising” and “including” are used in their open, non-limiting sense.

The term “alkyl”, as used herein, unless otherwise indicated, includes saturated monovalent hydrocarbon radicals having straight, branched, or cyclic moieties (including fused and bridged bicyclic and spirocyclic moieties), or a combination of the foregoing moieties. For an alkyl group to have cyclic moieties, the group must have at least three carbon atoms.

A “lower alkyl” is intended to mean an alkyl group having from 1 to 4 carbon atoms in its chain. The term “heteroalkyl” refers to a straight- or branched-chain alkyl group having from 2 to 12 atoms in the chain, one or more of which is a heteroatom selected from S, O, and N. Exemplary heteroalkyls include alkyl ethers, secondary and tertiary amines, alkyl sulfides and the like.

The term “alkenyl”, as used herein, unless otherwise indicated, includes alkyl moieties having at least one carbon-carbon double bond wherein alkyl is as defined above and including E and Z isomers of said alkenyl moiety.

The term “alkynyl”, as used herein, unless otherwise indicated, includes alkyl moieties having at least one carbon-carbon triple bond wherein alkyl is as defined above.

The term “carbocycle” refers to a saturated, partially saturated, unsaturated, or aromatic, monocyclic or fused or non-fused polycyclic, ring structure having only carbon ring atoms (no heteroatoms, i.e., non-carbon ring atoms). Exemplary carbocycles include cycloalkyl, aryl, and cycloalkyl-aryl groups.

The term “heterocycle” refers to a saturated, partially saturated, unsaturated, or aromatic, monocyclic or fused or non-fused polycyclic, ring structure having one or more heteroatoms selected from N, O, and S. Exemplary heterocycles include heterocycloalkyl, heteroaryl, and heterocycloalkyl-heteroaryl groups.

A “cycloalkyl group” is intended to mean a saturated or partially saturated, monocyclic, or fused or spiro polycyclic, ring structure having a total of from 3 to 18 carbon ring atoms (but no heteroatoms). Exemplary cycloalkyls include cyclopropyl, cyclobutyl, cyclopentyl, cyclopentenyl, cyclohexyl, cycloheptyl, adamantyl, and like groups.

A “heterocycloalkyl group” is intended to mean a monocyclic, or fused or spiro polycyclic, ring structure that is saturated or partially saturated, and has a total of from 3 to 18 ring atoms, including 1 to 5 heteroatoms selected from nitrogen, oxygen, and sulfur. Illustrative Examples of heterocycloalkyl groups include pyrrolidinyl, tetrahydrofuryl, piperidinyl, piperazinyl, morpholinyl, thiomorpholinyl, aziridinyl, and like groups.

The term “aryl”, as used herein, unless otherwise indicated, includes an organic radical derived from an aromatic hydrocarbon by removal of one hydrogen, such as phenyl or naphthyl.

The term “4-10 membered heterocyclic”, as used herein, unless otherwise indicated, includes aromatic and non-aromatic heterocyclic groups containing one to four heteroatoms each selected from O, S and N, wherein each heterocyclic group has from 4-10 atoms in its ring system, and with the proviso that the ring of said group does not contain two adjacent O or S atoms. Non-aromatic heterocyclic groups include groups having only 4 atoms in their ring system, but aromatic heterocyclic groups must have at least 5 atoms in their ring system. The heterocyclic groups include benzo-fused ring systems. An example of a 4 membered heterocyclic group is azetidinyl (derived from azetidine). An example of a 5 membered heterocyclic group is thiazolyl and an example of a 10 membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the groups listed above, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl (N-attached) or imidazol-3-yl (C-attached). An example of a heterocyclic group wherein 2 ring carbon atoms are substituted with oxo (═O) moieties is 1,1-dioxo-thiomorpholinyl.

A “heteroaryl group” is intended to mean a monocyclic or fused or spiro polycyclic, aromatic ring structure having from 4 to 18 ring atoms, including from 1 to 5 heteroatoms selected from nitrogen, oxygen, and sulfur. Illustrative Examples of heteroaryl groups include pyrrolyl, thienyl, oxazolyl, pyrazolyl, thiazolyl, furyl, pyridinyl, pyrazinyl, triazolyl, tetrazolyl, indolyl, quinolinyl, quinoxalinyl, benzthiazolyl, benzodioxinyl, benzodioxolyl, benzooxazolyl, and the like. The term “alkoxy”, as used herein, unless otherwise indicated, includes O-alkyl groups wherein alkyl is as defined above.

The term “amino” is intended to mean the —NH₂ radical. The term “halogen” represents chlorine, fluorine, bromine or iodine.

The term “halo”, as used herein, unless otherwise indicated, means fluoro, chloro, bromo or iodo. Preferred halo groups are fluoro, chloro and bromo.

The term “a pharmaceutically acceptable salt” refers to a salt that retains the biological effectiveness of the free acids and bases of the specified compound and that is not biologically or otherwise undesirable. A compound of the invention may possess a sufficiently acidic, a sufficiently basic, or both functional groups, and accordingly react with any of a number of inorganic or organic bases, and inorganic and organic acids, to form a pharmaceutically acceptable salt. Exemplary pharmaceutically acceptable salts include those salts prepared by reaction of the compounds of the present invention with a mineral or organic acid or an inorganic base, such as salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycollates, tartrates, methane-sulfonates, propanesulfonates, naphthalene-1-sulfonates, naphthalene-2-sulfonates, and mandelates.

The term “substituted” means that the specified group or moiety bears one or more substituents. The term “unsubstituted” means that the specified group bears no substituents. The term “optionally substituted” means that the specified group is unsubstituted or substituted by one or more substituents.

The term “SARS-inhibiting agent” means any rhinovirus protease inhibitor compound represented by formula I or a pharmaceutically acceptable salt, hydrate, prodrug, active metabolite or solvate thereof. Examples of rhinovirus protease inhibitors can be found in, but not limited to, U.S. application Ser. Nos. 09/301977 and 09/726376, which are incorporated herein in their entirety by reference.

The term “processes mediated by rhinovirus protease”, as used herein, refers to biological, physiological, endocrinological, and other bodily processes which are mediated by receptor or receptor combinations which are responsive to the rhinovirus inhibitors described herein (e.g., SARS-related virus). Modulation of such processes can be accomplished in vitro or in vivo. In vivo modulation can be carried out in a wide range of subjects, such as, for example, humans, rodents, sheep, pigs, cows, and the like.

The term “interfering with or preventing” SARS-related coronavirus (“SARS”) viral replication in a cell means to reduce SARS replication or production of SARS components necessary for progeny virus in a cell as compared to a cell not being transiently or stably transduced with the ribozyme or a vector encoding the ribozyme. Simple and convenient assays to determine if SARS viral replication has been reduced include an ELISA assay for the presence, absence, or reduced presence of anti-SARS antibodies in the blood of the subject (Nasoff et al., PNAS 88:5462-5466, 1991), RT-PCR (Yu et al., in Viral Hepatitis and Liver Disease 574-477, Nishioka, Suzuki and Mishiro (Eds.); Springer-Verlag Tokyo, 1994). Such methods are well known to those of ordinary skill in the art. Alternatively, total RNA from transduced and infected “control” cells can be isolated and subjected to analysis by dot blot or northern blot and probed with SARS specific DNA to determine if SARS replication is reduced. Alternatively, reduction of SARS protein expression can also be used as an indicator of inhibition of SARS replication. A greater than fifty percent reduction in SARS replication as compared to control cells typically quantitates a prevention of SARS replication.

The term “pharmaceutically acceptable carrier” refers to a carrier or adjuvant that may be administered to a patient, together with a compound of this invention, and which does not destroy the pharmacological activity thereof and is nontoxic when administered in doses sufficient to deliver a therapeutic amount of the compound.

The term “prodrug” is a compound that may be converted under physiological conditions or by solvolysis to the specified compound or to a pharmaceutically acceptable salt of such compound. A prodrug may be a derivative of one of the hydroxamate compounds of the present invention that contains a moiety, such as for example —CO₂R,—PO(OR)₂ or —C═NR, that may be cleaved under physiological conditions or by solvolysis. Any suitable R substituent may be used that provides a pharmaceutically acceptable solvolysis or cleavage product. A prodrug containing such a moiety may be prepared according to conventional procedures by treatment of a hydroxamate compound of this invention containing, for example, an amido, carboxylic acid, or hydroxyl moiety with a suitable reagent.

The term “active metabolite” refers to a pharmacologically active product produced through metabolism in the body of a specified hydroxamate compound or salt thereof.

Prodrugs and active metabolites of the inhibitor compounds described herein may be identified using routine techniques known in the art. See, e.g., Bertolini et al., J. Med. Chem., 40:2011-2016 (1997); Shan et al., J. Pharm. Sci., 86 (7):765-767 (1997); Bagshawe, Drug Dev. Res., 34:220-230 (1995); Bodor, Advances in Drug Res., 13:224-331 (1984); Bundgaard, “Design of Prodrugs” (Elsevier Press, 1985); Larsen, Design and Application of Prodrugs, Drug Design and Development (Krogsgaard-Larsen et al. eds., Harwood Academic Publishers, 1991); Dear et al., Chromatogr. B, 748:281-293 (2000); Spraul et al., J. Pharmaceutical & Biomedical Analysis, 10 (8):601-605 (1992); and Prox et al., Xenobiol, 3(2):103-112 (1992).

The term “solvate” is intended to mean a pharmaceutically acceptable solvate form of a specified compound that retains the biological effectiveness of such compound. Examples of solvates include compounds of the invention in combination with water, isopropanol, ethanol, methanol, DMSO, ethyl acetate, acetic acid, or ethanolamine.

If an inhibitor compound used in the method of the invention is a base, a desired salt may be prepared by any suitable method known to the art, including treatment of the free base with an inorganic acid (such as hydrochloric acid, hydrobromic acid, sulfuric acid, nitric acid, phosphoric acid, and the like), or with an organic acid (such as acetic acid, maleic acid, succinic acid, mandelic acid, fumaric acid, malonic acid, pyruvic acid, oxalic acid, glycolic acid, salicylic acid, pyranosidyl acid (such as glucuronic acid or galacturonic acid), alpha-hydroxy acid (such as citric acid or tartaric acid), amino acid (such as aspartic acid or glutamic acid), aromatic acid (such as benzoic acid or cinnamic acid), sulfonic acid (such as p-toluenesulfonic acid or ethanesulfonic acid), and the like.

If an inhibitor compound used in the method of the invention is an acid, a desired salt may be prepared by any suitable method known to the art, including treatment of the free acid with an inorganic or organic base (such as an amine (primary, secondary, or tertiary)), an alkali metal hydroxide, or alkaline earth metal hydroxide. Illustrative examples of suitable salts include organic salts derived from amino acids (such as glycine and arginine), ammonia, primary amines, secondary amines, tertiary amines, and cyclic amines (such as piperidine, morpholine, and piperazine), as well as inorganic salts derived from sodium, calcium, potassium, magnesium, manganese, iron, copper, zinc, aluminum, and lithium.

In the case of inhibitor compounds, prodrugs, salts, or solvates that are solids, it is understood by those skilled in the art that the hydroxamate compound, prodrugs, salts, and solvates used in the method of the invention, may exist in different polymorph or crystal forms, all of which are intended to be within the scope of the present invention and specified formulas. In addition, the hydroxamate compound, salts, prodrugs and solvates used in the method of the invention may exist as tautomers, all of which are intended to be within the broad scope of the present invention.

In some cases, the inhibitor compounds, salts, prodrugs and solvates used in the method of the invention may have chiral centers. When chiral centers are present, the hydroxamate compound, salts, prodrugs and solvates may exist as single stereoisomers, racemates, and/or mixtures of enantiomers and/or diastereomers. All such single stereoisomers, racemates, and mixtures thereof are intended to be within the broad scope of the present invention.

As generally understood by those skilled in the art, an optically pure compound is one that is enantiomerically pure. As used herein, the term “optically pure” is intended to mean a compound comprising at least a sufficient activity. Preferably, an optically pure amount of a single enantiomer to yield a compound having the desired pharmacological pure compound of the invention comprises at least 90% of a single isomer (80% enantiomeric excess), more preferably at least 95% (90% e.e.), even more preferably at least 97.5% (95% e.e.), and most preferably at least 99% (98% e.e.).

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above. In a preferred embodiment of the present invention, “treating” or “treatment” means at least the mitigation of a disease condition in a human, that is alleviated by the inhibition of the activity of one or more coronaviral 3C-like proteases, including, but not limited to the 3C-like protease of the causative agent for SARS. In the case of SARS, representative disease conditions include fever, dry cough, dyspnea, headache, hypoxemia, lymphopenia, elevated aminotransferase levels as well as viral titer. Methods of treatment for mitigation of a disease condition include the use of one or more of the compounds in the invention in any conventionally acceptable manner. According to certain preferred embodiments of the invention, the compound or compounds of the present invention are administered to a mammal, such as a human, in need thereof. Preferably, the mammal in need thereof is infected with a coronavirus such as the causative agent of SARS.

The present invention also includes prophylactic methods, comprising administering an effective amount of a compound of the invention, or a pharmaceutically acceptable salt, prodrug, pharmaceutically active metabolite, or solvate thereof to a mammal, such as a human, at risk for infection by a coronavirus. According to certain preferred embodiments, an effective amount of one or more compounds of the invention, or a pharmaceutically acceptable salt, prodrug, pharmaceutically active metabolite, or solvate thereof is administered to a human at risk for infection by the causative agent for SARS. The prophylactic methods of the invention include the use of one or more of the compounds in the invention in any conventionally acceptable manner.

Recent evidence indicates that a new coronavirus is the causative agent of SARS. The nucleotide sequence of the SARS-associated coronavirus has also recently been determined and made publically available.

The activity of the inhibitor compounds as inhibitors of SARS-related viral activity may be measured by any of the suitable methods available in the art, including in vivo and in vitro assays. The activity of the compounds of the present invention as inhibitors of coronavirus 3C-like protease activity (such as the 3C-like protease of the SARS coronavirus) may be measured by any of the suitable methods known to those skilled in the art, including in vivo and in vitro assays. Examples of suitable assays for activity measurements include the antiviral cell culture assays described herein as well as the antiprotease assays described herein, such as the assays described in Examples 1 through 3.

Administration of the inhibitor compounds and their pharmaceutically acceptable prodrugs, salts, active metabolites, and solvates may be performed according to any of the accepted modes of administration available to those skilled in the art. Illustrative Examples of suitable modes of administration include oral, nasal, pulmonary, parenteral, topical, transdermal, and rectal. Oral, intravenous, and nasal deliveries are preferred.

A SARS-inhibiting agent may be administered as a pharmaceutical composition in any suitable pharmaceutical form. Suitable pharmaceutical forms include solid, semisolid, liquid, or lyopholized formulations, such as tablets, powders, capsules, suppositories, suspensions, liposomes, and aerosols. The SARS-inhibiting agent may be prepared as a solution using any of a variety of methodologies. For example, the SARS-inhibiting agent can be dissolved with acid (e.g., 1 M HCl) and diluted with a sufficient volume of a solution of 5% dextrose in water (D5W) to yield the desired final concentration of SARS-inhibiting agent (e.g., about 15 mM). Alternatively, a solution of D5W containing about 15 mM HCl can be used to provide a solution of the SARS-inhibiting agent at the appropriate concentration. Further, the SARS-inhibiting agent can be prepared as a suspension using, for example, a 1% solution of carboxymethylcellulose (CMC).

Acceptable methods of preparing suitable pharmaceutical forms of the pharmaceutical compositions are known or may be routinely determined by those skilled in the art. For example, pharmaceutical preparations may be prepared following conventional techniques of the pharmaceutical chemist involving steps such as mixing, granulating, and compressing when necessary for tablet forms, or mixing, filling, and dissolving the ingredients as appropriate, to give the desired products for oral, parenteral, topical, intravaginal, intranasal, intrabronchial, intraocular, intraaural, and/or rectal administration.

Pharmaceutical compositions of the invention may also include suitable excipients, diluents, vehicles, and carriers, as well as other pharmaceutically active agents, depending upon the intended use. Solid or liquid pharmaceutically acceptable carriers, diluents, vehicles, or excipients may be employed in the pharmaceutical compositions. Illustrative solid carriers include starch, lactose, calcium sulfate dihydrate, terra alba, sucrose, talc, gelatin, pectin, acacia, magnesium stearate, and stearic acid. Illustrative liquid carriers include syrup, peanut oil, olive oil, saline solution, and water. The carrier or diluent may include a suitable prolonged-release material, such as glyceryl monostearate or glyceryl distearate, alone or with a wax. When a liquid carrier is used, the preparation may be in the form of a syrup, elixir, emulsion, soft gelatin capsule, sterile injectable liquid (e.g., solution), or a nonaqueous or aqueous liquid suspension.

A dose of the pharmaceutical composition may contain at least a therapeutically effective amount of an SARS-inhibiting agent and preferably is made up of one or more pharmaceutical dosage units. The selected dose may be administered to a mammal, for example, a human patient, in need of treatment mediated by inhibition of SARS-related coronavirus activity, by any known or suitable method of administering the dose, including topically, for example, as an ointment or cream; orally; rectally, for example, as a suppository; parenterally by injection; intravenously; or continuously by intravaginal, intranasal, intrabronchial, intraaural, or intraocular infusion. When the composition is administered in conjunction with a cytotoxic drug, the composition can be administered before, with, and/or after introduction of the cytotoxic drug. However, when the composition is administered in conjunction with radiotherapy, the composition is preferably introduced before radiotherapy is commenced.

The phrases “therapeutically effective amount” and “effective amount” are intended to mean the amount of an inventive agent that, when administered to a mammal in need of treatment, is sufficient to effect treatment for injury or disease conditions alleviated by the inhibition of SARS viral replication such as for potentiation of anti-cancer therapies or inhibition of neurotoxicity consequent to stroke, head trauma, and neurodegenerative diseases. The amount of a given SARS-inihibiting agent used in the method of the invention that will be therapeutically effective will vary depending upon factors such as the particular SARS-inihibiting agent, the disease condition and the severity thereof, the identity and characteristics of the mammal in need thereof, which amount may be routinely determined by artisans.

It will be appreciated that the actual dosages of the SARS-inhibiting agents used in the pharmaceutical compositions of this invention will be selected according to the properties of the particular agent being used, the particular composition formulated, the mode of administration and the particular site, and the host and condition being treated. Optimal dosages for a given set of conditions can be ascertained by those skilled in the art using conventional dosage-determination tests. For oral administration, e.g., a dose that may be employed is from about 0.001 to about 1000 mg/kg body weight, preferably from about 0.1 to about 100 mg/kg body weight, and even more preferably from about 1 to about 50 mg/kg body weight, with courses of treatment repeated at appropriate intervals.

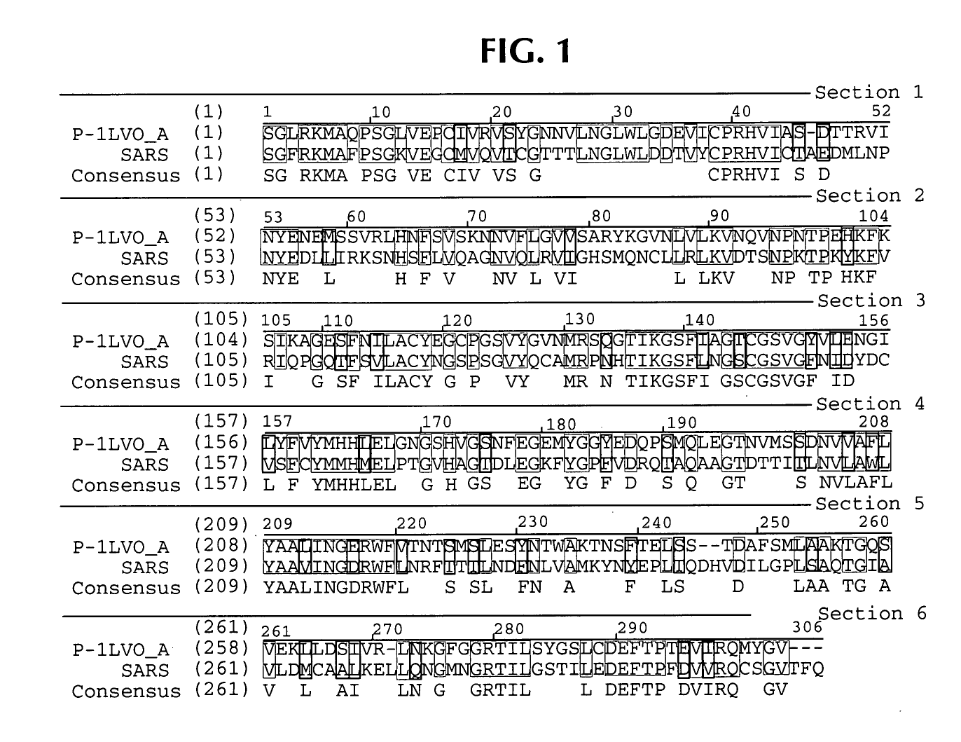

Protein functions required for coronavirus replication and transcription are encoded by the so-called “replicase” gene. Two overlapping polyproteins are translated from this gene and extensively processed by viral proteases. The C-proximal region is processed at eleven conserved interdomain junctions by the coronavirus main or “3C-like” protease. The name “3C-like” protease derives from certain similarities between the coronavirus enzyme and the well-known picornavirus 3C proteases. These include substrate preferences, use of cysteine as an active site nucleophile in catalysis, and similarities in their putative overall polypeptide folds. A comparison of the amino acid sequence of the SARS-associated coronavirus 3C-like protease to that of other known coronaviruses shows the amino acid sequence to be highly conserved, particularly in the catalytically important regions of the protease (FIG. 1).

Amino acids of the substrate in the protease cleavage site are numbered from the N to the C terminus as follows: -P3-P2-P1-P1′-P2′-P3′, with cleavage occurring between the P1 and P1′ residues (Schechter & Berger, 1967). Substrate specificity is largely determined by the P2, P1 and P1′ positions. Coronavirus main protease cleavage site specificities are highly conserved with a requirement for glutamine at P1 and a small amino acid at P1′ (Journal of General Virology 83, pp. 595-599 (2002)).

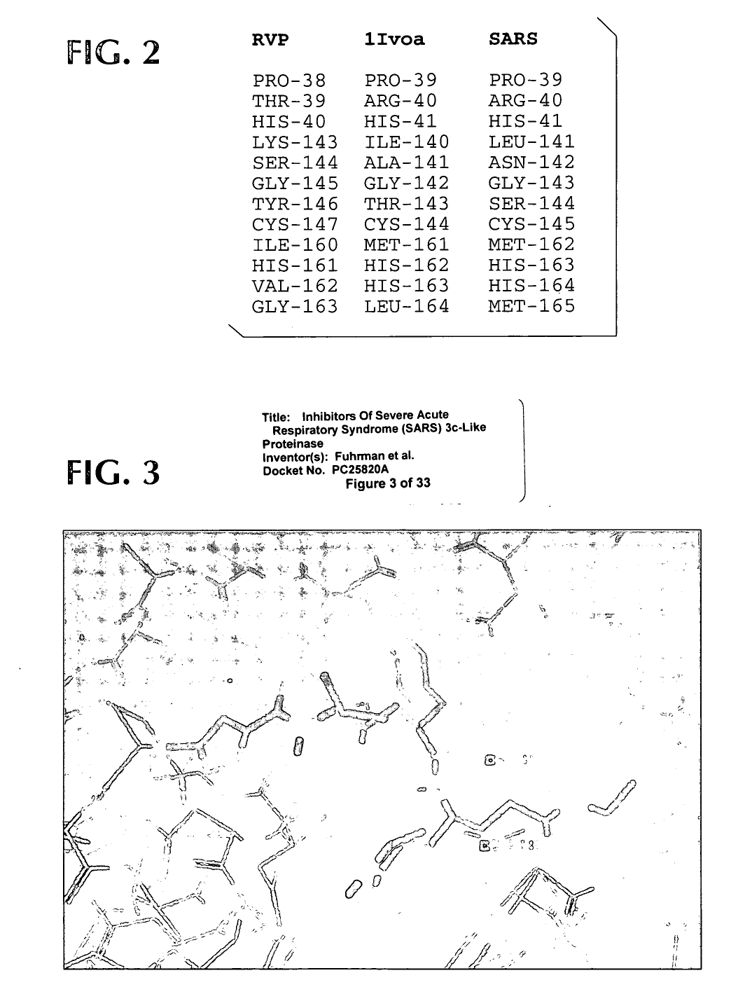

For almost 10 years, Pfizer-La Jolla has been engaged in an effort to discover and develop drugs with utility for treating the common cold by targeting a key enzyme in rhinovirus replication, namely the 3C protease. Potent, non-toxic agents with broad-spectrum activity against multiple rhinovirus serotypes have been identified. These compounds are described, for example, in U.S. Pat. Nos. 6,514,997, 6,531,452 and 5,962,487 each of which is incorporated herein in its entirety by reference. Recentiy, Hilgenfeld and colleagues published a high-resolution x-ray structure of the porcine transmissible gastroenteritis coronavirus main protease (The EMBO Journal, Vol. 21, pp. 3213-3224 (2002)). Atomic coordinates are available through the Protein Data Bank under accession code 1LVO. Our observations of the catalytic and structural similarities between rhinovirus 3C protease and coronavirus “3C-like” main protease, lead to the conclusion that selected inhibitors of rhinovirus 3C protease would be useful against the coronavirus main (3C-like) protease (FIG. 3).

Several considerations come into play when developing strategies for design of therapeutically efficacious serine and cysteine protease inhibitors. For many of these proteins, specificity pockets for substrate (or inhibitor) recognition are shallow, and binding determinants are widely dispersed over large surface areas. Difficulties inherent in discovering small molecules with high affinity for such binding sites are in many respects analogous to those encountered in attempting to disrupt proteinBprotein interactionswith small effector molecules. Serine proteases such as factor Xa and thrombin, proteins involved in the blood-coagulation pathway with deep well defined S1 specificity pockets, have been targeted effectively with structurally diverse, small, noncovalent inhibitors and thus are exceptions to this generalization (19). However, for virally encoded serine and cysteine proteases of known structure, such as the herpes family of sedne proteases, hepatitis C NS3 protease, picornavirus 3C proteases and coronaviral 3C-like proteases, the fact that substrate recognition is modulated by extensive proteinBprotein interactions represents a significant impediment for design of specific inhibitors.

Peptidic substrates in which the scissile amide carbonyl is replaced by a Michael acceptor were first introduced as specific irreversible inhibitors of the cysteine protease papain by Hanzlik and coworkers (20, 21). We reasoned that, although this reaction is probably facilitated by the especially nucleophilic thiolateimidazolium ion pair in papain-like cysteine proteases, suitably activated Michael acceptors might also undergo addition by the presumably less nucleophilic catalytic cysteine of 3C and 3C-like proteases.

Covalent irreversible inactivation of 3C and 3C-like proteases by Michael acceptors proceeds according to a kinetic mechanism that can be broken down into two parts.

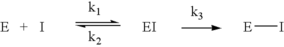

The inhibitor initially forms a reversible encounter complex with 3C, which can then undergo a chemical step (nucleophilic attack by the reactive site Cys) leading to stable covalent-bond formation. The observed second-order rate constant for inactivation (k_{obs}/l) depends on both the equilibrium binding constant k₂/k₁ and the chemical rate for covalent bond formation k₃ (Meara, J. P. & Rich, D. H. (1995) Bioorg. Med. Chem. Lett. 5, 2277-2282). We anticipated that Michael-acceptor inhibitors with specificity for 3C-like protease, as with 3C protease, would likely achieve high rates of enzyme inactivation by combining good equilibrium binding with a modest rate of covalent-bond formation. The rate of chemical inactivation presumably depends on not only the intrinsic electrophilic character of the inhibitor, but on how the reactive vinyl group is oriented relative to the Cys in the reactive site before nucleophilic attack and on the extent to which the transition state for the reaction can be stabilized by the enzyme. Mechanism-based activation of an inherently weak Michael acceptor as a means of increasing the rate of the chemical step, and thus k_{obs}/l, is conceptually more attractive than attempting to achieve a similar effect by simply increasing intrinsic electrophilic reactivity, which would likely impart undesirable properties to such compounds.

### EXAMPLES

In the examples described below, unless otherwise indicated, all temperatures are set forth in degrees Celsius and all parts and percentages are by weight. Reagents may be purchased from commercial suppliers, such as Sigma-Aldrich Chemical Company, or Lancaster Synthesis Ltd. and may be used without further purification unless otherwise indicated. Tetrahydrofuran (THF) and N,N-dimethylformamide (DMF) may be purchased from Aldrich in Sure Seal bottles and used as received. All solvents may be purified using standard methods known to those skilled in the art, unless otherwise indicated.

The SARS coronavirus protease inhibitors as used in the method of the present invention can be prepared as described in U.S. patent application Ser. Nos. 09/301977 and 09/726376 (Dragovich et al.), each of which is incorporated herein in their entirety by reference.

Preferred compounds in accordance with the invention may be prepared in manners analogous to those specifically described below.

### Example 1

Protection from Infection

The ability of compounds to protect cells against infection by the SARS coronavirus is measured by a cell viability assay similar to that described in Weislow, O. S., Kiser, R., Fine, D. L., Bader,J., Shoemaker, R. H., and Boyd, M. R. 1989. New Soluble-Formazan Assay for HIV-1 Cytopathic Effects: Application to High-Flux Screening of Synthetic and Natural Products for AIDS-Antiviral Activity. Journal of the National Cancer Institute 81(08):577-586), utilizing neutral red staining as an endpoint. Briefly, Vero cells are resuspended in medium containing appropriate concentrations of compound or medium only. Cells are infected with SARS-associated virus or mock-infected with medium only. One to seven days later, neutral red is added to the test plates and following incubation at 37° C. for one hour, cells are solubilized and the amount of neutral red produced is quantified spectrophotometrically at 540 nm. Data is expressed as the percent of neutral red produced in compound-treated cells compared to neutral red produced in wells of uninfected, compound-free cells. The fifty percent effective concentration (EC50) is calculated as the concentration of compound that increases the percent of neutral red production in infected, compound-treated cells to 50% of that produced by uninfected, compound-free cells. The 50% cytotoxicity concentration (CC50) is calculated as the concentration of compound that decreases the percentage of neutral red produced in uninfected, compound-treated cells to 50% of that produced in uninfected, compound-free cells. The therapeutic index is calculated by dividing the cytotoxicity (CC50) by the antiviral activity (EC50).

### Example 2

Viral Yield Assay

The ability of compounds to protect cells by infection is evaluated in a virus yield assay similar to that described in A. K. Patick, S. L. Binford, M. A. Brothers, R. L. Jackson, C. E. Ford, M. D. Diem, F. Maldonado, P. S. Dragovich, R. Zhou, T. J. Prins, S. A. Fuhrman, J. W. Meador, L. S. Zalman, D. A. Matthews and S. T. Worland. 1999. In vitro antiviral activity of AG7088, a potent inhibitor of human rhinovirus 3C protease. Antimicrob. Agents and Chemo. 43:2444-2450. Briefly, 0.2 ml of serial ten-fold dilutions of SARS-associated virus is allowed to adsorb onto monolayers of Vero cells. After one hour adsorption, the cell monolayers are washed twice with PBS and overlayed with medium containing 0.5% Seaplaque agarose (FMC Bioproducts, Rockland, Me.). After one to seven days of incubation at 34° C., the cell monolayers are fixed with EAF (65% ethanol, 22% acetic acid, and 4% formaldehyde), stained with 1% crystal violet and virus plaques enumerated. Data is expressed as plaque forming units (PFU) per ml. The fifty percent EC50 is calculated as the concentration of compound that decreases the number of PFU/ml in infected, compound-treated cells to 50% of that produced by infected, compound-free cells.

### Example 3

Coronavirus 3C Protease FRET Assay and Analysis

Proteolytic activity of Coronavirus 3C protease is measured using a continuous fluorescence resonance energy transfer assay. The substrate, DABCYL-GRAVFQGPVG-EDANS, is prepared by modification of the core decapeptide (American Peptide Systems) and purified prior to use by HPLC using a C-18 resin (Alltech). Other peptide cores are possible and may, for example, be derived from protease cleavage sites in the published sequence of the SARS coronavirus. Preferred peptides retain the P1 and P1′ amino acids (QG) of the above decapeptide (the proteolytic cleavage site). In addition, other fluorescent probe/quencher combinations are possible. The assays include reaction buffer (50 mM Tris, pH 7.5, 1 mM EDTA 0.1 to 10 □M substrate, 5 to 50 nM coronavirus 3C protease, 2% DMSO and inhibitor as appropriate. Cleavage of the DABCYL-EDANS substrate peptide is monitored by the appearance of fluorescent emission at 490 nm (following excitation at 336 nm). Data are analyzed with the non-linear regresssion analysis program Kalidagraph using the equation:

FU=offset+(limit)(1−e^{-(kobs)t})

where offset equals the fluorescence signal of the uncleaved peptide substrate, and limit equals the fluorescence of fully cleaved peptide substrate. The kobs is the first order rate constant for this reaction, and in the absence of any inhibitor represents the utilization of substrate. In an enzyme start reaction which contains an irreversible inhibitors, and where the calculated limit is less than 20% of the theoretical maximum limit, the calculated kobs represents the rate of inactivation of coronavirus 3C protease. The slope (kobs/l) of a plot of kobs vs. [I] is a measure of the avidity of the inhibitor for an enzyme. For very fast irreversible inhibitors, kobs/l is calculated from observations at only one or two [I] rather than as a slope.

### Example 4

Structure-assisted Selection of Michael acceptor-based Inhibitors of 3C-like Protease Inhibitors

Homology Modeling

A homology model for SARS 3C-like protease was created using the atomic coordinates for the recently published coronavirus “3C-like” protease as a template. BLAST was employed to identify the 3C-like proteinase from the genomic RNA sequence of SARS (AY274119). Minor adjustment to the BLAST output resulted in an alignment with high percent identity and few gaps (FIG. 1), and this alignment was used to create a homology model with the MODELLER package in Insight2000 (Sanchez and Sali 2000).

Twelve residues with high structural conservation (FIG. 2) were identified by visual inspection of the rhinovirus 3C (1CQQ) and TGEV 3C-like proteinase (1LVO) structures, as well as the SARS 3C-like proteinase homology model. The structures were superimposed in a common reference frame by minimizing the root mean square difference (RMSD) between the backbone atoms of these residues, with RMSD<0.6 Angstroms². Inspection of the structures in the common reference frame demonstrates strong conservation of the side-chain conformations of the catalytic cysteine and histidine residues (FIG. 3).

Modeling Analysis

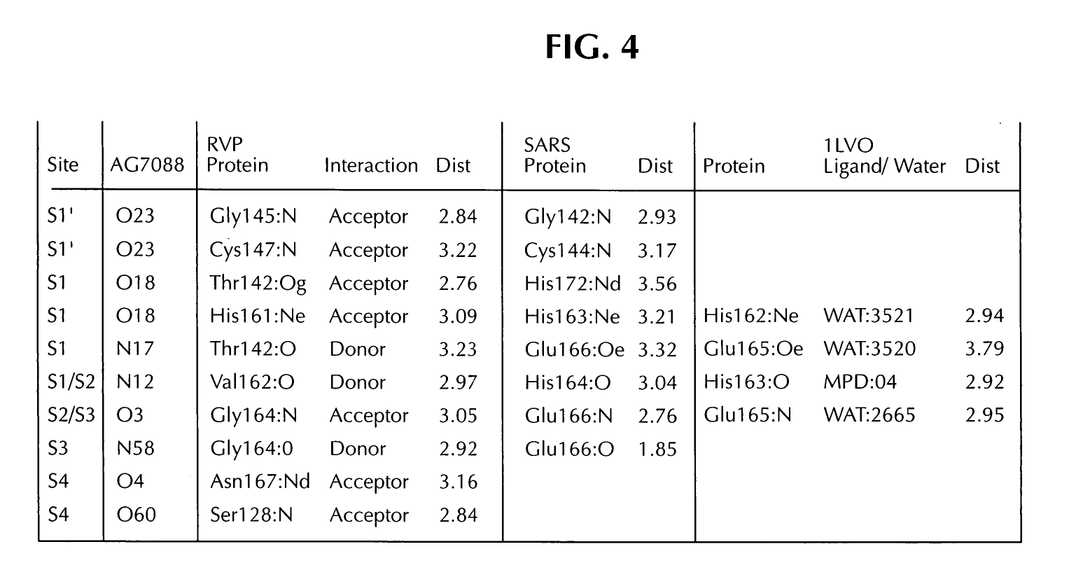

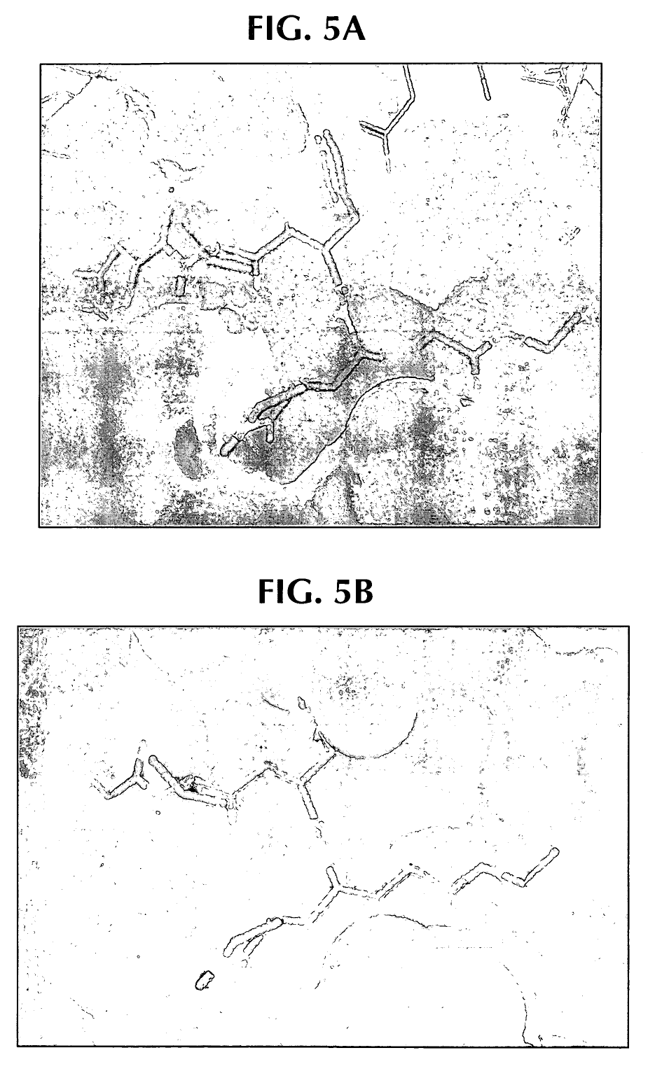

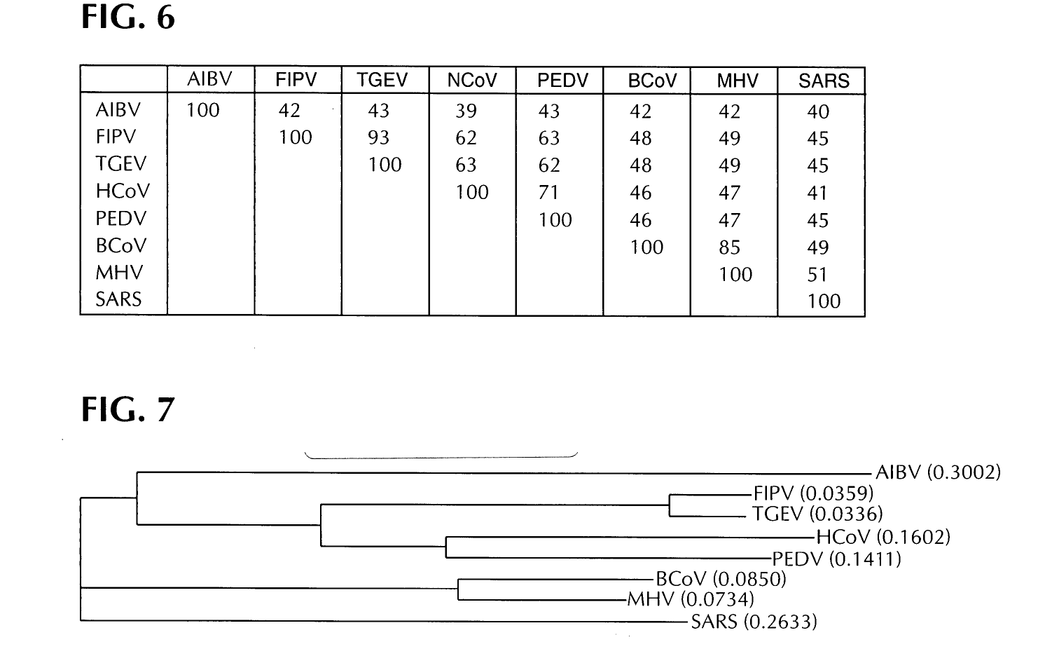

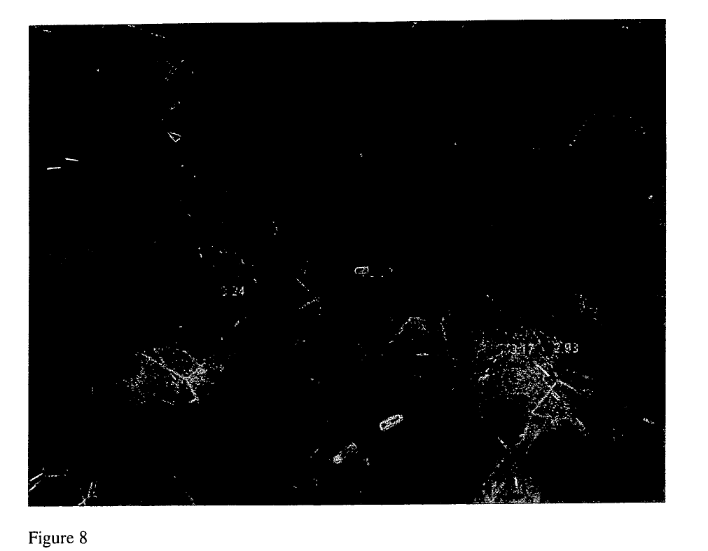

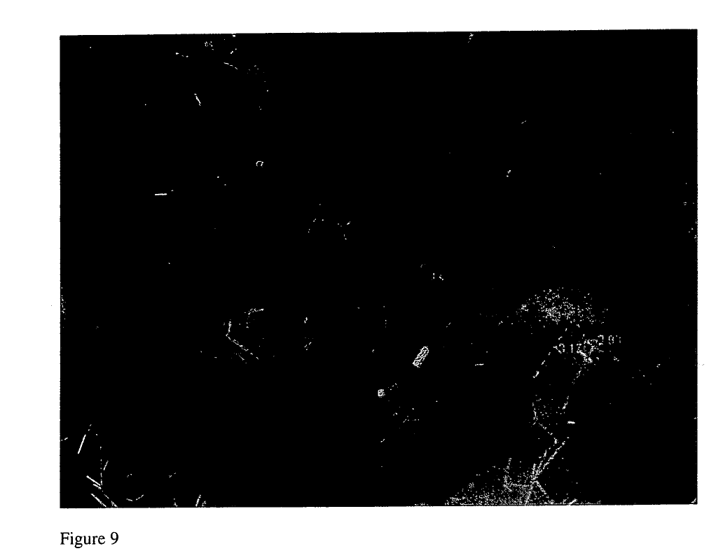

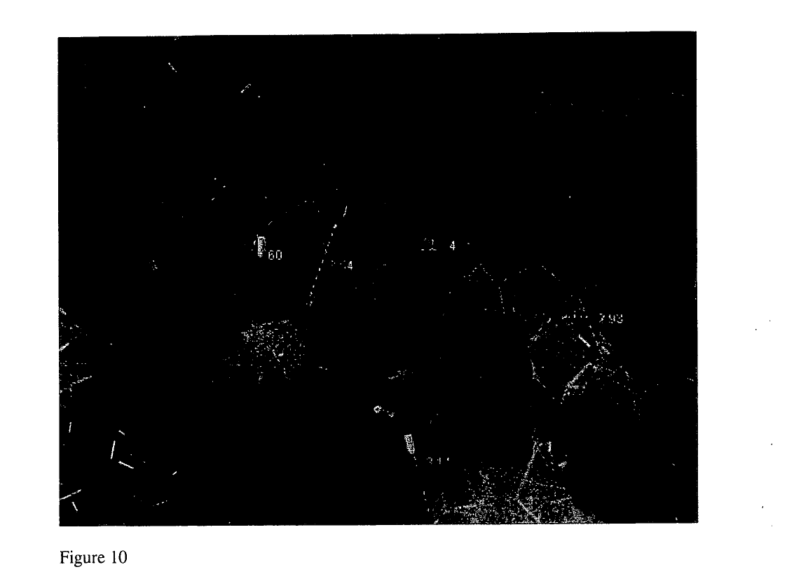

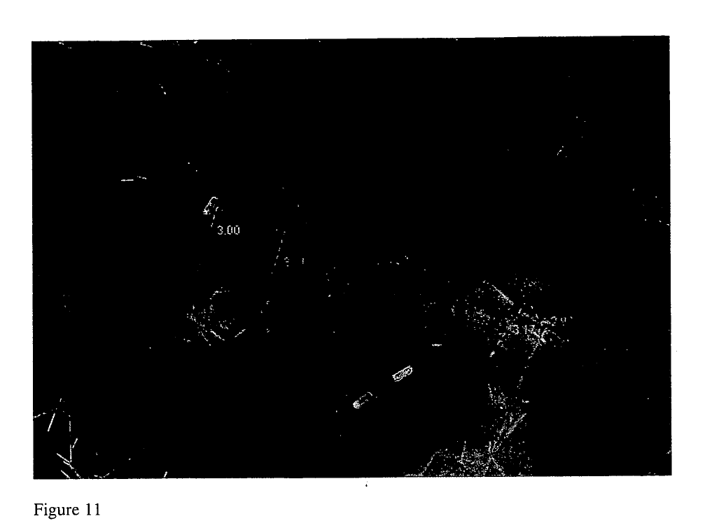

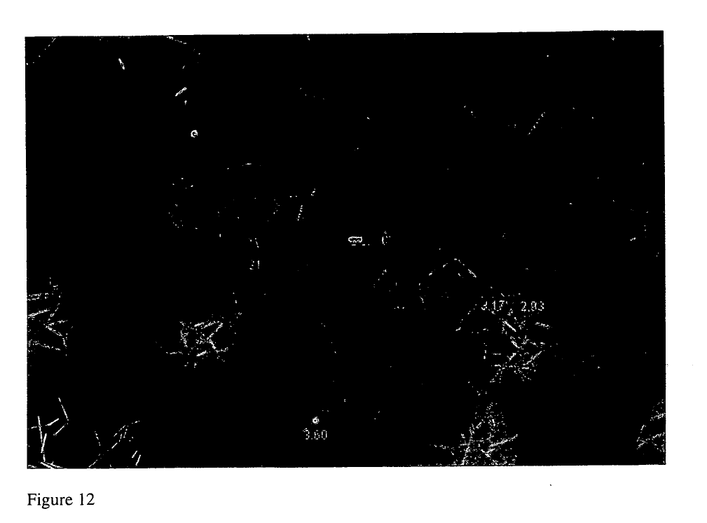

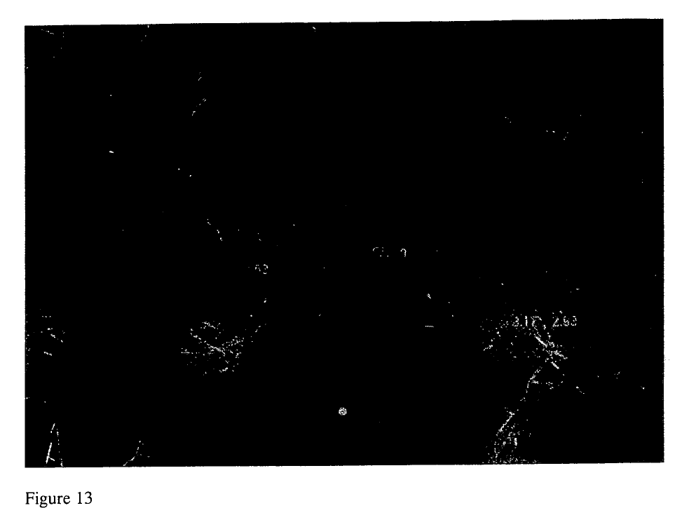

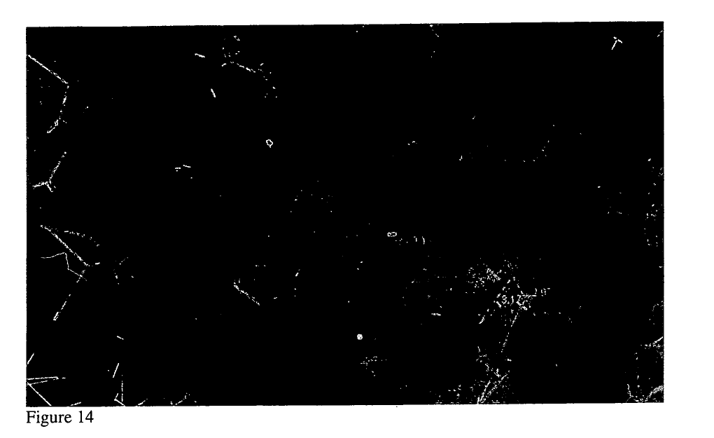

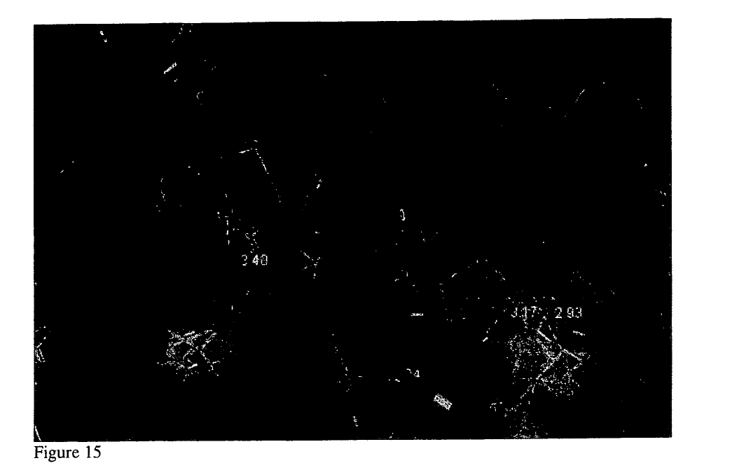

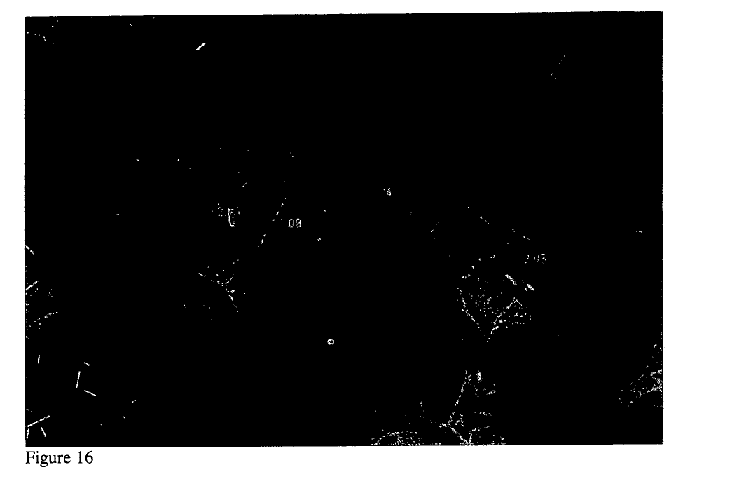

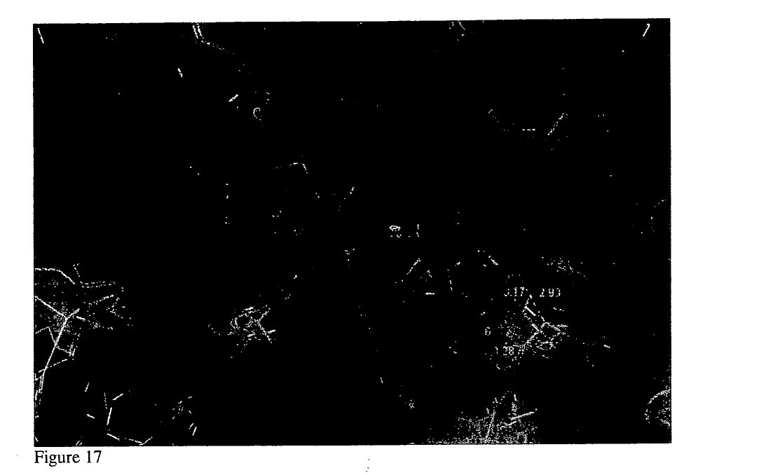

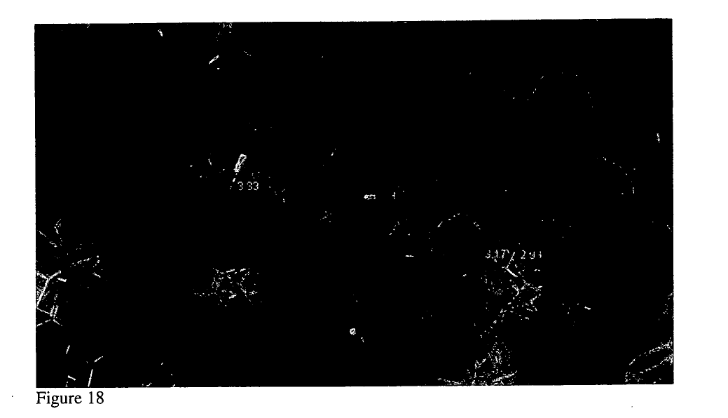

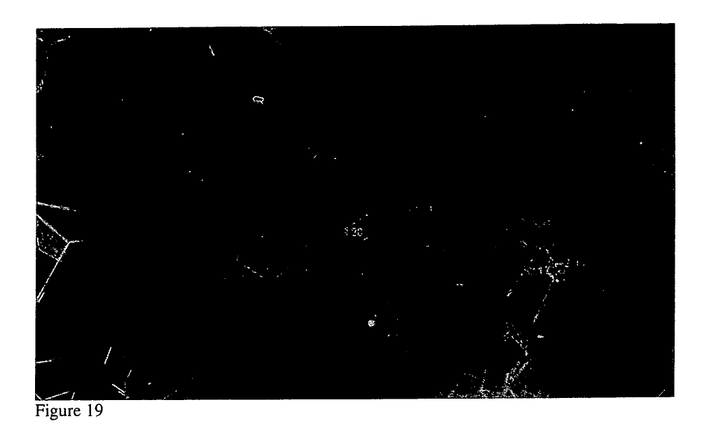

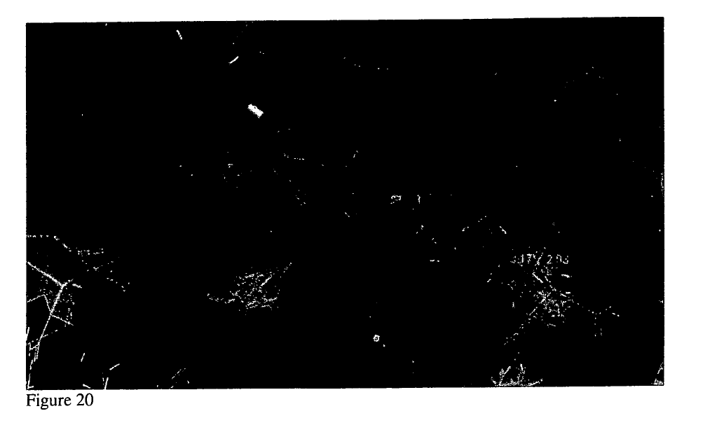

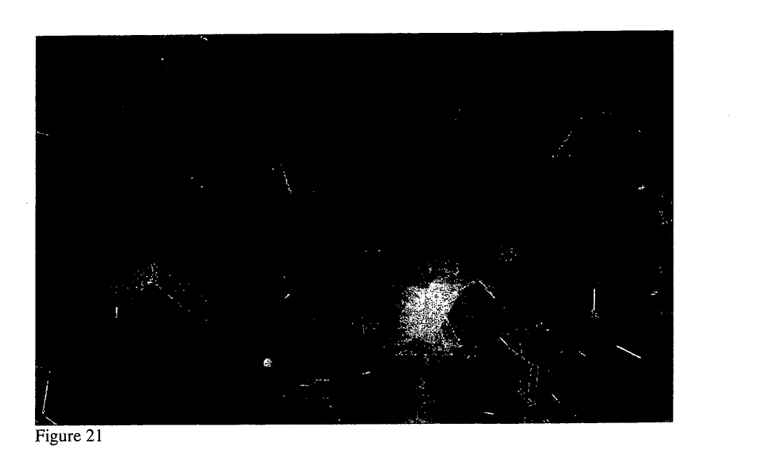

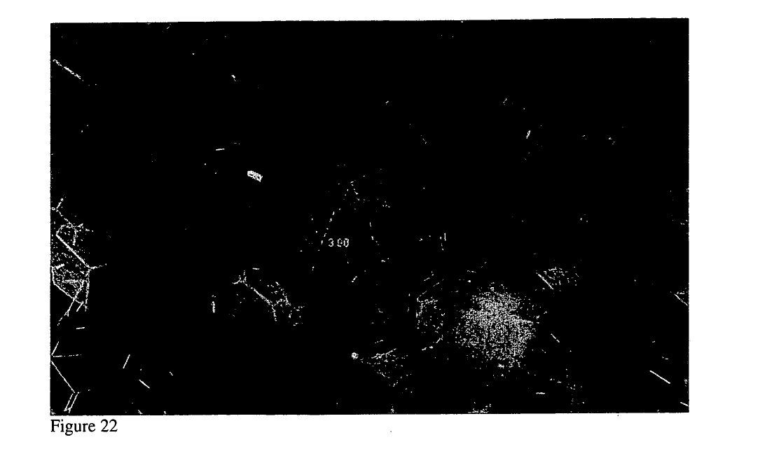

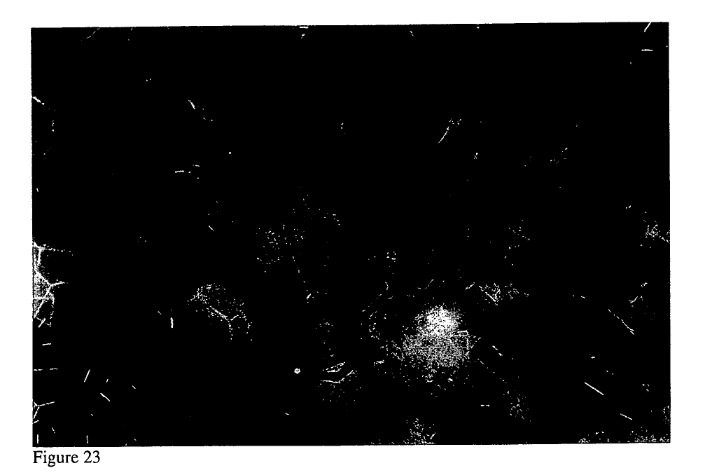

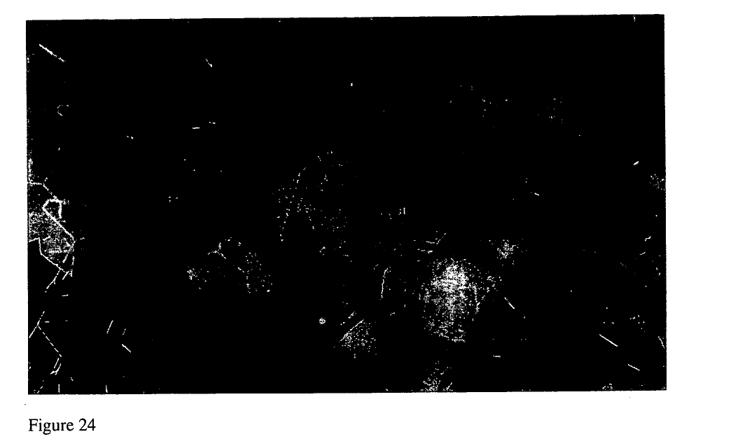

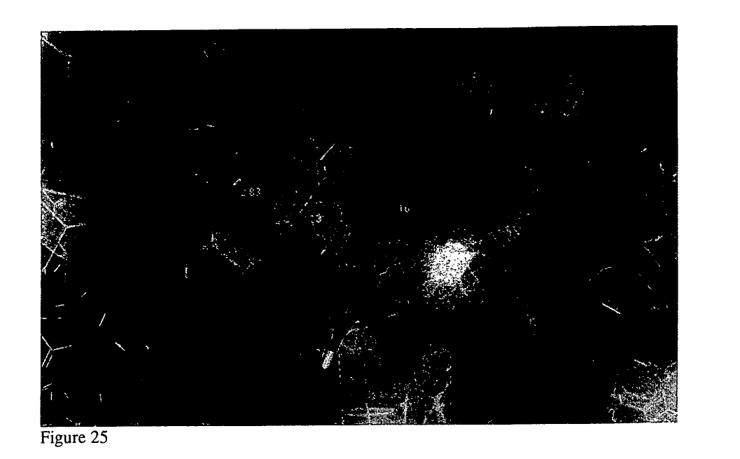

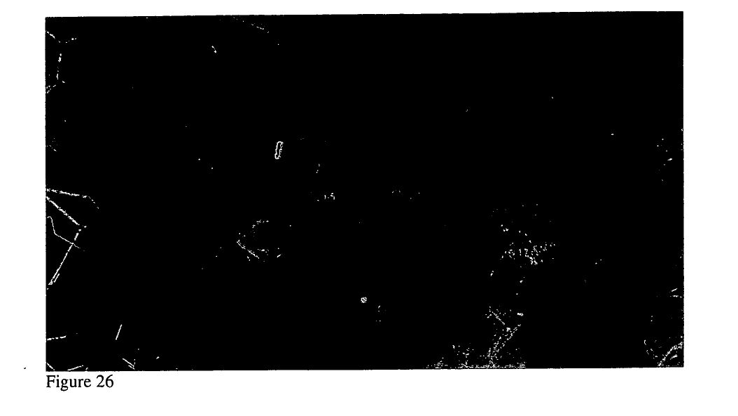

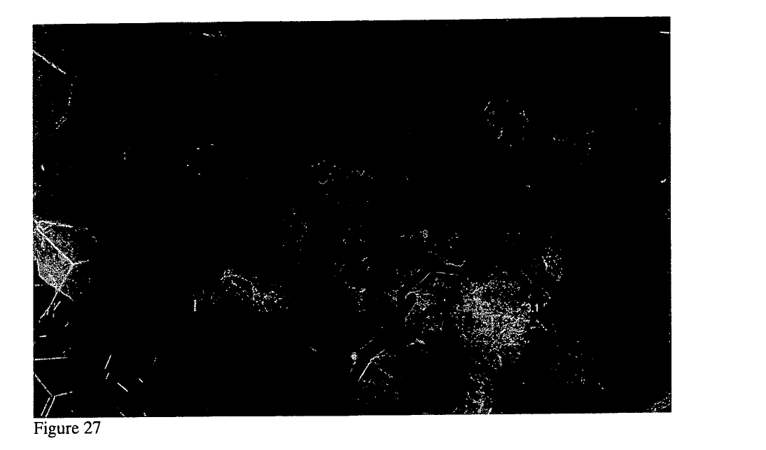

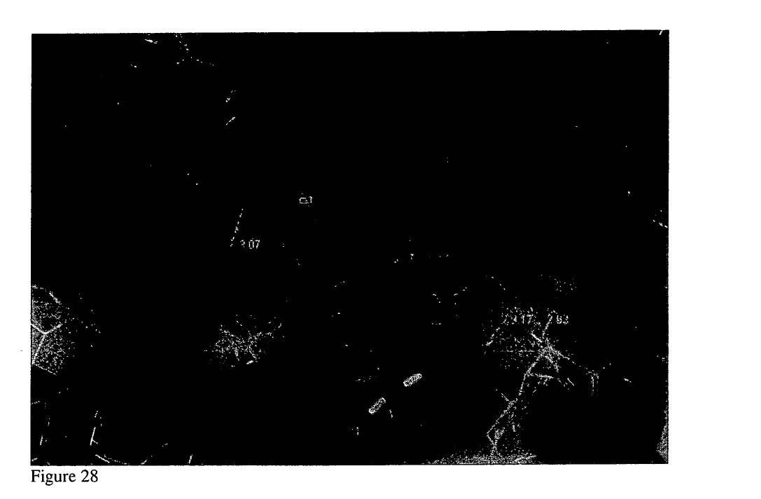

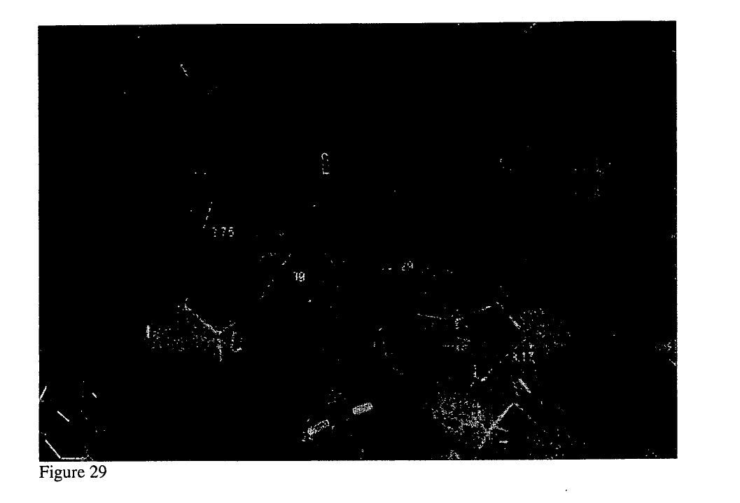

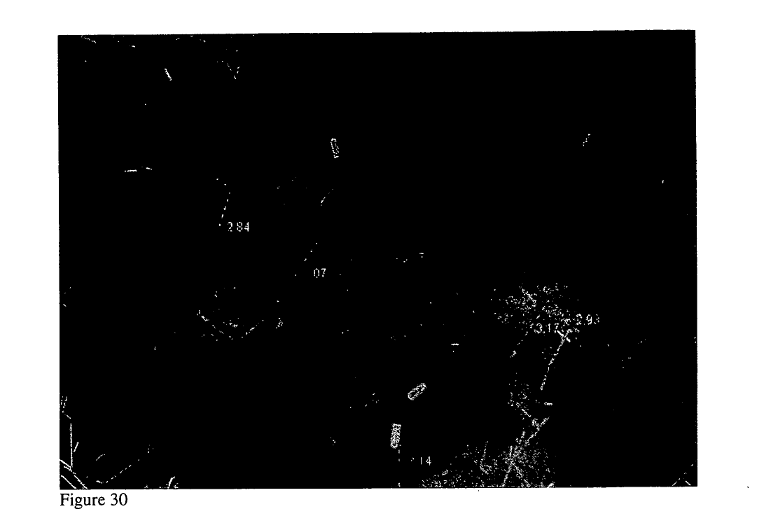

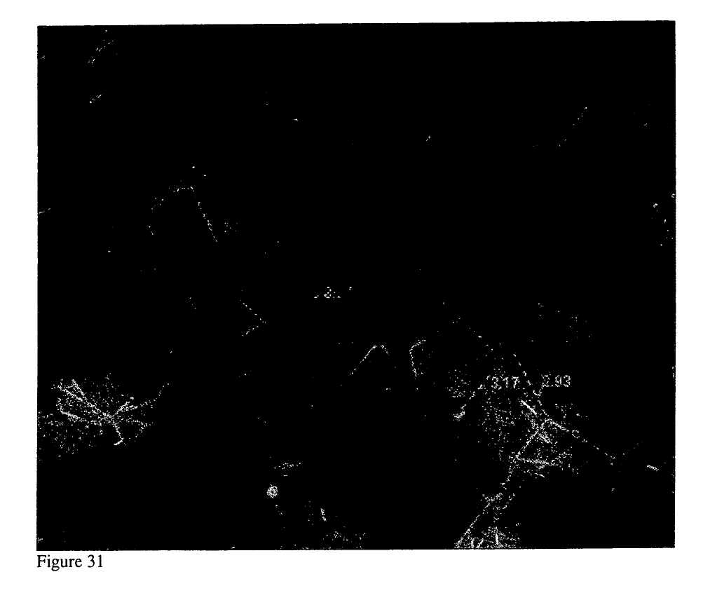

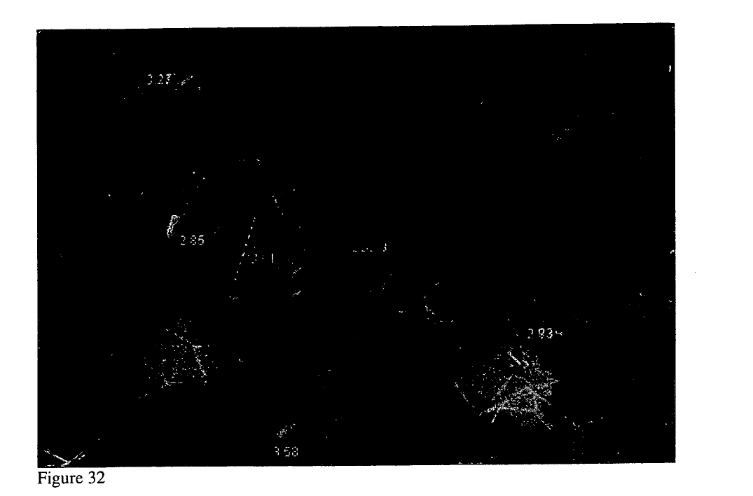

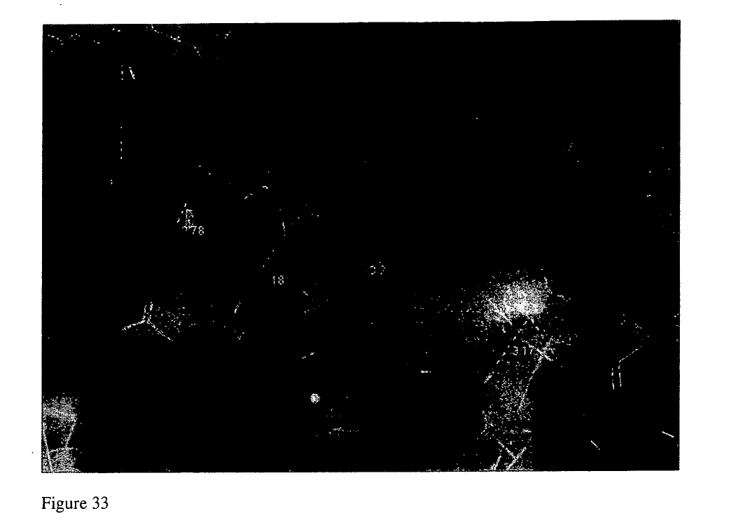

Electronic and steric characteristics of coronavirus “3C-like” protease near the active site cysteine and the adjacent S1 and S1′ specificity pockets are similar to those of rhinovirus 3C protease with corresponding features closely aligned based on the structural superposition described above. In the S1′ specificity pocket, main-chain nitrogens Gly145 and Cys147 activate the carbonyl oxygen in AG7088. The sequence and structural location of these two residues are conserved in the TGEV structure (Gly142 and Cys144). In the S1 specificity pocket, there are three hydrogen bonds between AG7088 and rhinovirus 3C protease (FIG. 4). These three hydrogen bonds are preserved in the SARS model, one of them involving a corresponding His Nitrogen in the two proteins, and the others substituted with alternate residues. Despite substitutions in the sequence of the S1 pocket, the solvent accessible surfaces of rhinovirus 3C protease and the SARS model have considerable agreement in the P1 binding site (FIG. 5). Further examination of the superposed structures indicates that an inhibitor such as AG7088 could all seven of the hydrogen bonds with coronavirus “3C-like” protease at P1, P2, and P3 that are observed for rhinovirus 3C protease (FIG. 4). Differences between the structures are most prevalent in the S3 and S4 pockets, suggesting that optimal inhibitors of rhinovirus 3C protease and SARS will differ in this region. Furthermore, the S2 specificity pocket is more constrained in the coronavirus protease, suggesting that inhibitors having side chains smaller than fluorophenyalanine (as in AG7088) could be preferred. This is consistent with the prevalence of Leu in many of the known coronavirus cleavage site sequences (Hegyi and Ziebuhr 2002). Coronavirus main protease cleavage site specificities are highly conserved with a requirement for glutamine at P1 and a small amino acid at P1′ (Hegyi and Ziebuhr 2002). Picornavirus 3C proteases also favor cleavage sites with glutamine at P1 and either Gly or Ala at P1′. The structural superposition described above indicates that the two proteins differ considerably in exactly how their respective S4 specificity pockets are constructed. The polypeptide chain loops that form S4 are also positioned differently relative to S1, S2, and S3 in the two viral proteases.

The modeling analysis leads to the following suggestions for inhibitors:

1. Michael acceptor based inhibitors with appropriate specificity elements should covalently inactivate coronavirus “3C-like” protease with both methyl and ethyl ester containing compounds.

2. Compounds with glutamine or the lactam side chain at P1 should be chosen.

3. Compounds with differing substituents at P2 should be selected including phe but also smaller side chains such as leu and val.

4. Wide variability should be acceptable at P3 as this side chain site is fully solvent accessible.

5. Size and conformational flexibility at P4 may be important. Smaller is probably better than larger based on modeling. Include thiocarbamate containing analogs.

Michael acceptor containing SARS protease inhibitor compounds are selected based on the above qualitative criteria. Alternatively, one may also dock available compounds to a homology model of the SARS protease. Such a model could be constructed using the known structure of porcine coronavirus protease and the gene sequence of the SARS virus “3C-like” protease.

### Example 5

Michael acceptor-based Inhibitors of the SARS Protease

Michael acceptor-based inhibitors having the criteria discussed above are assayed using the protease and antiviral assays described above in Examples 1-3. The following compounds are identified as inhibitors of the 3C-like protease of the SARS-associated virus.

Table 1 below provides examples of inhibitor compounds that are useful as SARS-related 3C protease inhibitors. However, the invention is not limited to these particular examples.

TABLE 1

| No. | MOL STRUCTURE | CHEM NAME | MOL FORMULA | MOL Wt |
|---|---|---|---|---|
| 1 | 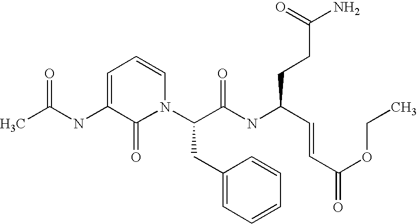 | 4-[2-(3-acetylamino-2-oxo-2H-pyridin- 1-yl)-3-phenypropionylamino]-6- carbamoylhex-2-enoic acid ethyl ester | C25 H30 N4 O6 | 482.534 |
| 2 | 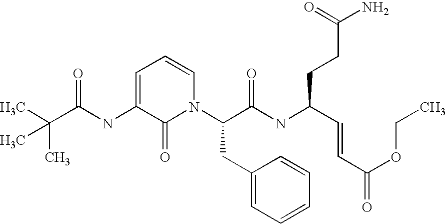 | 6-carbamoyl-4-{□2-[3-(2,2- dimethylpropionylamino)-2-oxo-2H- pyridin-1-yl]-3-phenylpropionylamino}- hex-2-enoic acid ethyl ester | C28 H36 N4 O6 | 524.614 |
| 3 | 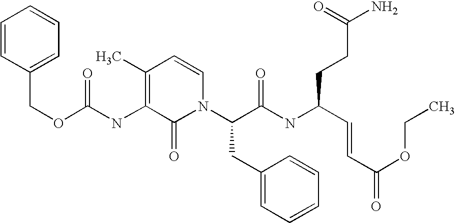 | 4-[2-(3-benzyloxycarbonylamino-4- methyl-2-oxo-2H-pyridin-1-yl)-3- phenylpropionylamino-6- carbamoylhex-2-enoic acid ethyl ester | C32 H36 N4 O7 | 588.657 |
| 4 | 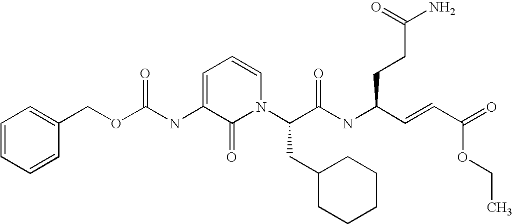 | 4-[2-(3-Benzyloxycarbonylamino-2- oxo-2H-pyridin-1-yl)-3-cyclohexyl- propionylamino]-6-carbamoyl-hex-2- enoic acid ethyl ester | C31 H40 N4 O7 | 580.678 |
| 5 | 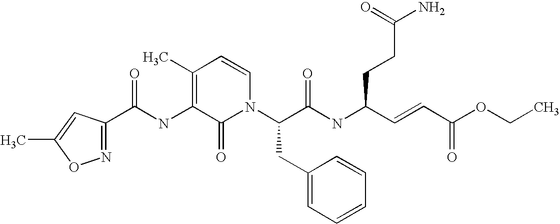 | 6-Carbamoyl-4-(2-{4-methyl-3-[(5- methyl-isoxazole-3-carbonyl)-amino]-2- oxo-2H-pyridin-1-yl}-3-phenyl- propionylamino)-hex-2-enoic acid ethyl ester | C29 H33 N5 O7 | 563.608 |
| 6 | 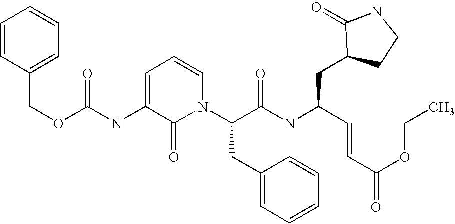 | 4-[2-(3-benzyloxycarbonylamino-2-oxo- 2H-pyridin-1-yl)-3- phenylpropionylamino]-5-(2- oxopyrrolidin-3-yl)-pent-2-enoic acid ethyl ester | C33 H36 N4 O7 | 600.668 |
| 7 | 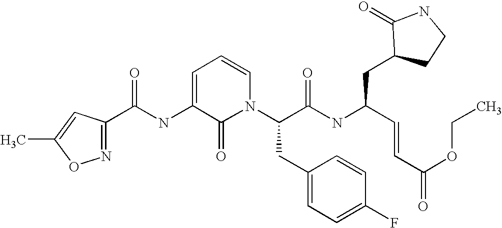 | 4-(3-(4-fluorophenyl)-2-{3-[(5- methylisoxazole-3-carbonyl)-amino]-2- oxo-2H-pyridin-1-yl}-propionylamino)-5- (2-oxopyrrolidin-3-yl)-pent-2-enoic acid ethyl ester | C30 H32 F N5 O7 | 593.609 |
| 8 | 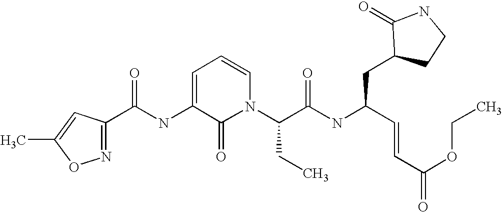 | 4-(2-{3-[(5-methylisoxazole-3- carbonyl)-amino]-2-oxo-2H-pyridin-1- yl}-butyrylamino)-5-(2-oxopyrrolidin-3- yl)-pent-2-enoic acid ethyl ester | C25 H31 N5 O7 | 513.548 |
| 9 | 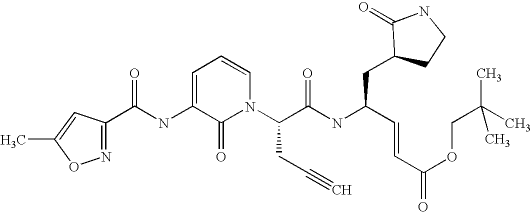 | 4-(2-{3-[(5-Methyl-isoxazole-3- carbonyl)-amino]-2-oxo-2H-pyridin-1- yl}-pent-4-ynoylamino)-5-(2-oxo- pyrrolidin-3-yl)-pent-2-enoic acid 2,2- dimethyl-propyl ester | C29 H35 N5 O7 | 565.623 |
| 10 | 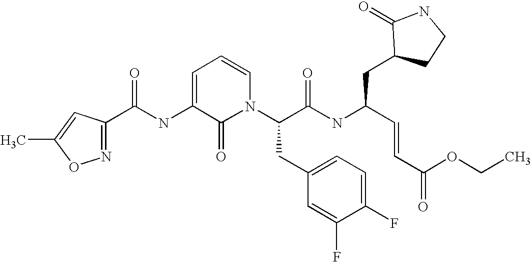 | (E)-(S)-4-[(S)-3-(3,4-Difluoro-phenyl)-2- (3-{[1-(5-methyl-isoxazol-3-yl)- methanoyl]-amino}-2-oxo-2H-pyridin-1- yl)-propanoylamino]-5-(2-oxo- pyrrolidin-3-yl)-pent-2-enoic acid ethyl ester | C30 H31 F2 N5 O7 | 611.599 |
| 11 | 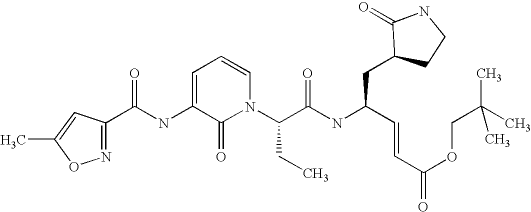 | (E)-(S)-4-[(S)-2-(3-{[1-(5-Methyl- isoxazol-3-yl)-methanoyl]-amino}-2- oxo-2H-pyridin-1-yl)-butanoylamino]-5- ((S)-2-oxopyrrolidin-3-yl)-pent-2-enoic acid 2,2-dimethyl-propyl ester | C28 H37 N5 O7 | 555.628 |
| 12 | 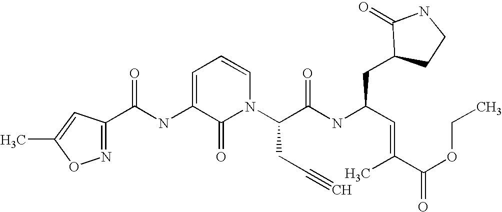 | (E)-(S)-2-methyl-4-[(S)-2-(3-{[1-(5- methylisoxazol-3-yl)-methanoyl]- amino}-2-oxo-2H-pyridin-1-yl)-pent-4- ynoylamino]-5-((S)-2-oxopyrrolidin-3- yl)-pent-2-enoic acid ethyl ester | C27 H31 N5 O7 | 537.57 |
| 13 | 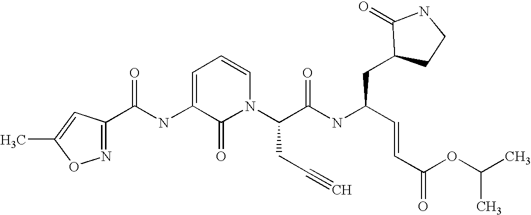 | (E)-(S)-4-[(S)-2-(3-{[1-(5-Methyl- isoxazol-3-yl)-methanoyl]-amino}-2- oxo-2H-pyridin-1-yl)-pent-4- ynoylamino]-5-((S)-2-oxo-pyrrolidin-3- yl)-pent-2-enoic acid isopropyl ester | C27 H31 N5 O7 | 537.57 |
| 14 | 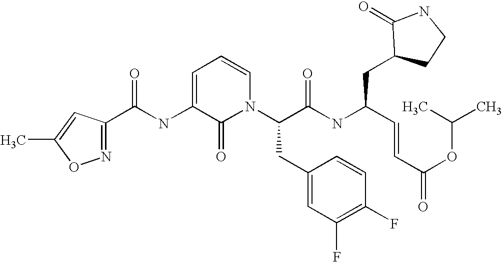 | (E)-(S)-4-[(S)-3-(3,4-Difluorophenyl)-2- (3-{[1-(5-methylisoxazol-3-yl)- methanoyl]-amino}-2-oxo-2H-pyridin-1- yl)-propanoylamino]-5-((S)-2- oxopyrrolidin-3-yl)-pent-2-enoic acid isopropyl ester | C31 H33 F2 N5 O7 | 625.626 |
| 15 | 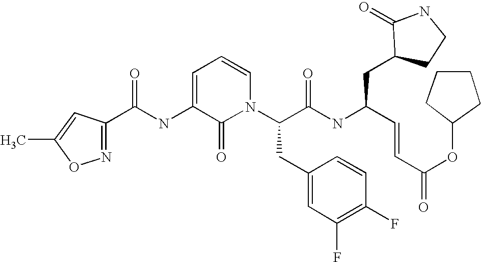 | (E)-(S)-4-[(S)-3-(3,4-Difluorophenyl)-2- (3-{[1-(5-methylisoxazol-3-yl)- methanoyl]-amino}-2-oxo-2H-pyridin-1- yl)-propanoylamino]-5-((S)-2- oxopyrrolidin-3-yl)-pent-2-enoic acid cyclopentyl ester | C33 H35 F2 N5 O7 | 651.664 |
| 16 | 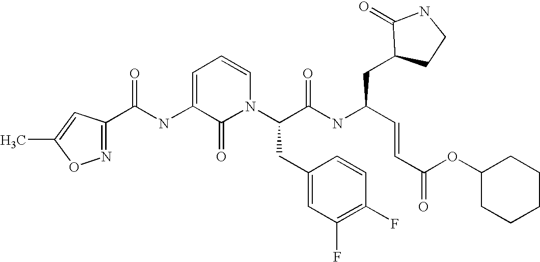 | (E)-(S)-4-[(S)-3-(3,4-Difluorophenyl)-2- (3-{[1-(5-methylisoxazol-3-yl)- methanoyl]-amino}-2-oxo-2H-pyridin-1- yl)-propanoylamino]-5-((S)-2- oxopyrrolidin-3-yl)-pent-2-enoic acid cyclohexyl ester | C34 H37 F2 N5 O7 | 665.69 |
| 17 | 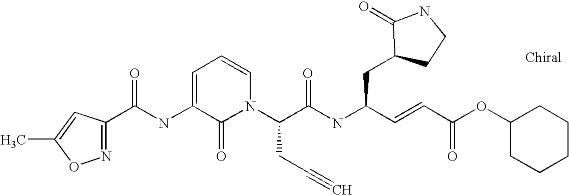 | (E)-4-[2-(3-{[1-(5-Methyl-isoxazol-3-yl)- methanoyl]-amino}-2-oxo-2H-pyridin-1- yl)-pent-4-ynoylamino]-5-(2-oxo- pyrrolidin-3-yl)-pent-2-enoic acid cyclohexyl ester | C30 H35 N5 O7 | 577.635 |
| 18 | 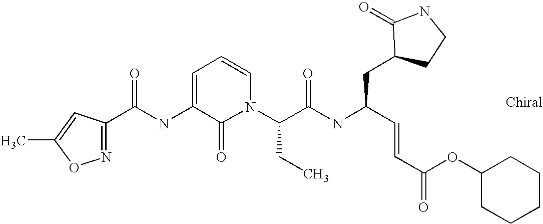 | (E)-4-[2-(3-{[1-(5-Methyl-isoxazol-3-yl)- methanoyl]-amino}-2-oxo-2H-pyridin-1- yl)-butanoylamino]-5-(2-oxo-pyrrolidin- 3-yl)-pent-2-enoic acid cyclohexyl ester | C29 H37 N5 O7 | 567.639 |
| 19 | 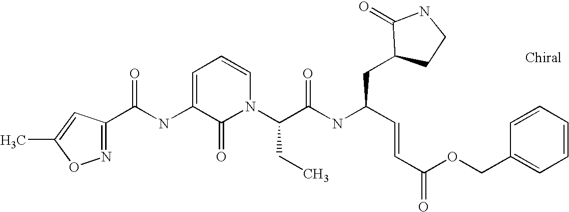 | (E)-4-[2-(3-{[1-(5-Methyl-isoxazol-3-yl)- methanoyl]-amino}-2-oxo-2H-pyridin-1- yl)-butanoylamino]-5-(2-oxo-pyrrolidin- 3-yl)-pent-2-enoic acid benzyl ester | C30 H33 N5 O7 | 575.619 |
| 20 | 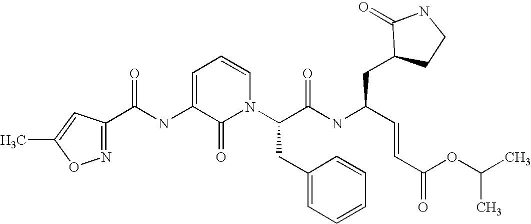 | (E)-(S)-4-[(S)-2-(3-{[1-(5-Methyl- isoxazol-3-yl)-methanoyl]-amino}-2- oxo-2H-pyridin-1-yl)-3-phenyl- propanoylamino]-5-((S)-2-oxo- pyrrolidin-3-yl)-pent-2-enoic acid isopropyl ester | C31 H35 N5 O7 | 589.646 |
| 21 | 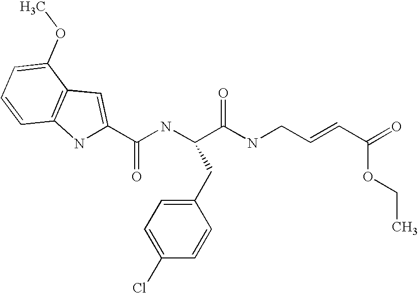 | 6-Carbamoyl-4-{3-(4-chloro-phenyl)-2- [(4-methoxy-1H-indole-2-carbonyl)- amino]-propionylamino}-hex-2-enoic acid ethyl ester | C28 H31 Cl N4 O6 | 555.028 |
| 22 | 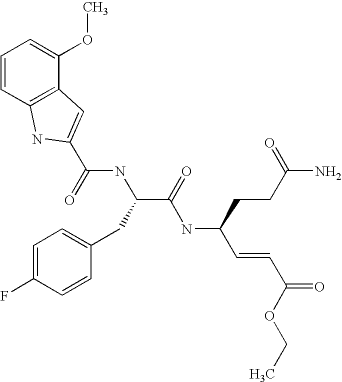 | 6-Carbamoyl-4-{3-(4-fluoro-phenyl)-2- [(4-methoxy-1H-indole-2-carbonyl)- amino]-propionylamino}-hex-2-enoic acid ethyl ester | C28 H31 F N4 O6 | 538.573 |
| 23 | 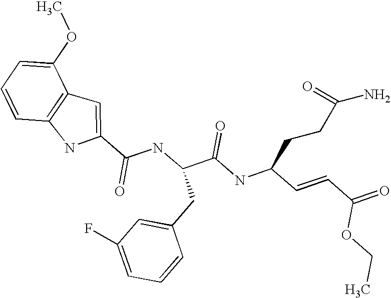 | 6-Carbamoyl-4-{3-(3-fluoro-phenyl)-2- [(4-methoxy-1H-indole-2-carbonyl)- amino]-propionylamino}-hex-2-enoic acid ethyl ester | C28 H31 F N4 O6 | 538.573 |
| 24 | 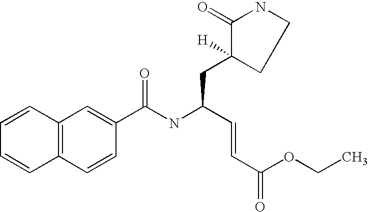 | (E)-(S)-4-[(1-Naphthalen-2-yl- methanoyl)-amino]-5-((S)-2-oxo- pyrrolidin-3-yl)-pent-2-enoic acid ethyl ester | C22 H24 N2 O4 | 380.442 |
| 25 | 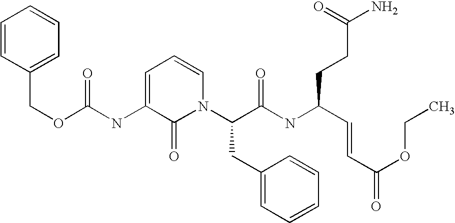 | 4-[2-(3-benzyloxycarbonylamino-2-oxo- 2H-pyridin-1-yl)-3- phenylpropionylamino]-6- carbamoylhex-2-enoic acid ethyl ester | C31 H34 N4 O7 | 574.631 |
| 26 | 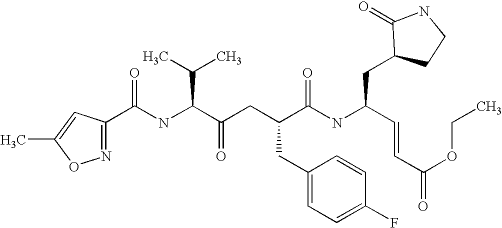 | 4-{2-(4-fluorobenzyl)-6-methyl-5-[(5- methylisoxazole-3-carbonyl)-amino]-4- oxoheptanoylamino}-5-(2-oxopyrrolidin- 3-yl)-pent-2-enoic acid ethyl ester | C31 H39 F N4 O7 | 598.668 |

While the invention has been described in terms of various preferred embodiments and specific examples, the invention should be understood as not being limited by the foregoing detailed description, but as being defined by the appended claims and their equivalents.

What is claimed is:

1. A method of interfering with or preventing SARS related coronavirus viral replication activity comprising contacting a SARS related coronavirus protease with a therapeutically effective amount of a rhinovirus 3C protease inhibitor.

2. A method according to claim 1, wherein said inhibitor is administered orally, intravenously or by inhalation.

3. A pharmaceutical composition for the treatment of SARS related cornoavirus in a mammal comprising an amount of a rhinovirus inhibitor that is effective in treating SARS related coronavirus and a pharmaceutically acceptable carrier.

4. A method according to claim 1 utilizing an inhibitor of formula l: 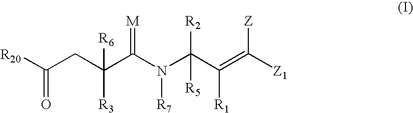
wherein
M is O or S;
R₁ is H, F, an alkyl group, OH, SH, or an O-alkyl group;
R₂ and R₅ are independently selected from H, 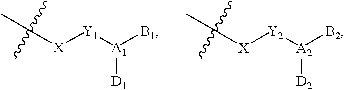
or an alkyl group, wherein said alkyl group is different from 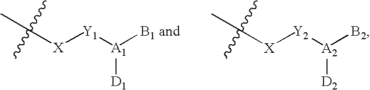
with the proviso that at least one of R₂ or R₅ must be 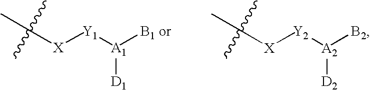
and wherein, when R₂ or R₅ is 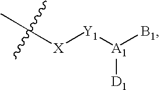
X is ═CH or ═CF and Y, is ═CH or ═CF,
or X and Y₁ together with Q′ form a three-membered ring in which Q′ is —C(R₁₀)(R₁₁)— or —O—, X is —CH— or —CF—, and Y₁ is —CH—, —CF—, or —C(alkyl)—, where R₁₀ and R₁₁ independently are H, a halogen, or an alkyl group, or, together with the carbon atom to which they are attached, form a cycloalkyl group or a heterocycloalkyl group,
or X is —CH₂—, —CF₂—, —CHF—, or —S—, and Y₁ is —O—, —S—, —NR₁₂—, —C(R₁₃)(R₁₄)—, —C(O)—, —C(S)—, or —C(CR₁₃R₁₄)—,
wherein R₁₂ is H or alkyl, and R₁₃ and R₁₄ independently are H, F, or an alkyl group, or, together with the atoms to which they are bonded, form a cycloalkyl group or a heterocycloalkyl group;
A₁ is C, CH, CF, S, P, Se, N, NR₁₅, S(O), Se(O), P—OR₁₅, or P—NR₁₅R₁₆,
wherein R₁₅ and R₁₆ independently are an alkyl group, a cycloalkyl group, a heterocycloalkyl group, an aryl group, or a heteroaryl group, or, together with the atom to which they are bonded, form a heterocycloalkyl group;
D₁ is a moiety with a lone pair of electrons capable of forming a hydrogen bond; and
B₁ is H, F, an alkyl group, a cycloalkyl group, a heterocycloalkyl group, an aryl group, a heteroaryl group, —OR₁₇, —SR₁₇, —NR₁₇R₁₈, —NR₁₉NR₁₇R₁₈, or —NR₁₇OR₁₈,
wherein R₁₇, R₁₈, and R₁₉ independently are H, an alkyl group, a cycloalkyl group, a heterocycloalkyl group, an aryl group, a heteroaryl group, or an acyl group;
and with the provisos that when D₁ is the moiety □N with a lone pair of electrons capable of forming a hydrogen bond, B₁ does not exist; and when A₁ is an sp³ carbon, B₁ is not —NR₁₇R₁₈ when D₁ is the moiety —NR₂₅R₂₆ with a lone pair of electrons capable of forming a hydrogen bond, wherein R₂₅ and R₂₆ are independently H, an alkyl group, a cycloalkyl group, a heterocycloalkyl group, an aryl group, or a heteroaryl group;
and wherein D₁-A₁-B₁ optionally forms a nitro group where A₁ is N; and further wherein, when R₂ or R₅ is 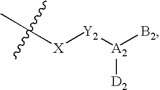
X is ═CH or ═CF and Y₂ is ═C, ═CH, or ═CF,
or X and Y₂ together with Q′ form a three-membered ring in which Q′ is —C(R₁₀)(R₁₁)— or —O—, X is —CH— or —CF—, and Y₂ is —CH—, —CF—, or —C(alkyl)—, where R₁₀ and R₁₁ independently are H, a halogen, or an alkyl group, or, together with the carbon atom to which they are attached, form a cycloalkyl group or a heterocycloalkyl group,
or X is —CH₂—, —CF₂—, —CHF—, or —S—, and Y₂ is —O—, —S—, —N(R′₁₂)—, —C(O)—, —C(R′₁₃)(R′₁₄)—, —C(S)—, or —C(CR′₁₃R′₁₄)—,
wherein R′₁₂ is H, an alkyl group, a cycloalkyl group, a heterocycloalkyl group, an aryl group, a heteroaryl group, —OR′₁₃, —NR′₁₃R′₁₄, —C(O)—R′₁₃, —SO₂R′₁₃, or —C(S)R′₁₃, and R′₁₃ and R′₁₄, independently are H, F, or an alkyl group, a cycloalkyl group, a heterocycloalkyl group, an aryl group, or a heteroaryl group, or, together with the atom to which they are attached, form a cycloalkyl group or a heterocycloalkyl group;
A₂ is C, CH, CF, S, P, Se, N, NR₁₅, S(O), Se(O), P—OR₁₅, or P—NR₁₅R₁₆, wherein R₁₅ and R₁₆ independently are an alkyl group, a cycloalkyl group, a heterocycloalkyl group, an aryl group, or a heteroaryl group, or, together with the atom to which they are bonded, form a heterocycloalkyl group;
D₂ is a moiety with a lone pair of electrons capable of forming a hydrogen bond; and
B₂ is H, F, an alkyl group, a cycloalkyl group, a heterocycloalkyl group, an aryl group, a heteroaryl group, —OR₁₇, —SR₁₇, —NR₁₇R₁₈, —NR₁₉NR₁₇R₁₈, or —NR₁₇OR₁₈,
wherein R₁₇, R₁₈, and R₁₉ independently are H, an alkyl group, a cycloalkyl group, a heterocycloalkyl group, an aryl group, a heteroaryl group, or an acyl group;
and further wherein any combination of Y₂, A₂, B₂, and D₂ optionally can form a cycloalkyl group, a heterocycloalkyl group, an aryl group, or a heteroaryl group;
R₃ and R₆ are independently H, F, an alkyl group, a cycloalkyl group, a heterocycloalkyl group, an aryl group, a heteroaryl group, —C(O)R₁₇, —OR₁₇, —SR₁₇, —NR₁₇R₁₈, —NR₁₉NR₁₇R₁₈, or —NR₁₇OR₁₈,
wherein R₁₇, R₁₈, and R₁₉ independently are H, an alkyl group, a cycloalkyl group, a heterocycloalkyl group, an aryl group, a heteroaryl group, or an acyl group;
or, R₃ and R₆, together with the carbon atom to which they are attached, form a cycloalkyl group or a heterocycloalkyl group;
R₇ is H, an alkyl group, a cycloalkyl group, a heterocycloalkyl group, an aryl group, a heteroaryl group, —OR₁₇, —SR₁₇, —NR₁₇R₁₈, —NR₁₉NR₁₇R₁₈, or —NR₁₇OR₁₈,
wherein R₁₇, R₁₈, and R₁₉ independently are H, an alkyl group, a cycloalkyl group, a heterocycloalkyl group, an aryl group, a heteroaryl group, or an acyl group;
or R₇, together with R₃ or R₆ and the atoms to which they are attached, forms a heterocycloalkyl group;
R₂₀ is H, OH, or any suitable organic moiety; and
Z and Z₁ are independently H, F, an alkyl group, a cycloalkyl group, a heterocycloalkyl group, an aryl group, a heteroaryl group, —C(O)R₂₁, —CO₂R₂₁, —CN, —C(O)NR₂₁,R₂₂, —C(O)NR₂₁OR₂₂, —C(S)R₂₁, —C(S)NR₂₁R₂₂, —NO₂, —SOR₂₁, —SO₂R₂₁, —SO₂NR₂₁R₂₂, —SO(NR₂₁)(OR₂₂), —SONR₂₁, —SO₃R₂₁, —PO(OR₂₁)₂, —PO(R₂₁)(R₂₂), —PO(NR₂₁R₂₂)(OR₂₃), PO(NR₂₁R₂₂)(NR₂₃R₂₄), —C(O)NR₂₁NR₂₂R₂₃, or —C(S)NR₂₁NR₂₂R₂₃,
wherein R₂₁, R₂₂, R₂₃, and R₂₄ are independently H, an alkyl group, a cycloalkyl group, a heterocycloalkyl group, an aryl group, a heteroaryl group, an acyl group, or a thioacyl group, or wherein any two of R₂₁, R₂₂, R₂₃, and R₂₄, together with the atom(s) to which they are bonded, form a heterocycloalkyl group;
or Z₁, as defined above, together with R₁, as defined above, and the atoms to which Z₁ and R₁ are bonded, form a cycloalkyl or heterocycloalkyl group,
or Z and Z₁, both as defined above, together with the atoms to which they are bonded, form a cycloalkyl or heterocycloalkyl group;
or a pharmaceutically acceptable prodrug, salt, active metabolite, or solvate thereof;
and wherein said compound, or pharmaceutically acceptable prodrug, salt, active metabolite, or solvate thereof, has antipicornaviral activity with an EC₅₀ less than or equal to 10 μM in a HI-HeLa cell culture assay.

5. A method according to claim 1 utilizing a rhinovirus inhibitor of the formula II: 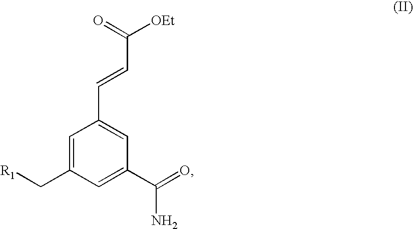
wherein R₁ is: 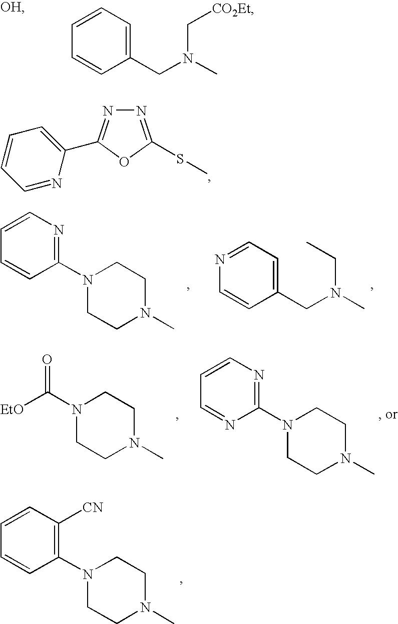
or a pharmaceutically acceptable salt, solvate, prodrug, or pharmaceutically active metabolite thereof.

6. A method according to claim 1 utilizing a rhinovirus inhibitor of the formula IIB: 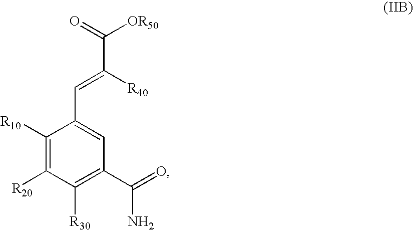
wherein:
R₁₀ is H or CH₃;
R₂₀ is H, OH, CH₂OH, or OCH₂Ph;
R₃₀ is H, OH, or OCH₂Ph;
R₄₀ is H or CN; and
R₅₀ is CH₂CH₃, CH₃, CH₂Ph, CH₂CH₂Ph, CH₂CH₂OH, or CH₂(2-pyridyl);
or a pharmaceutically acceptable salt, solvate, prodrug, or pharmaceutically active metabolite thereof.

7. A method according to claim 1 utilizing a rhinovirus inhibitor of the formula IIC: 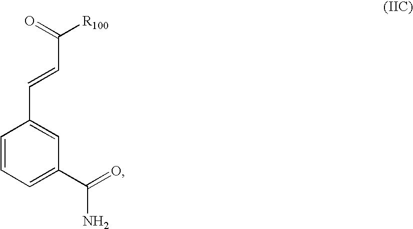
wherein R₁₀₀ is CH₃, phenyl, Ph(4-NCH₃), Ph(4-OCH₃), 2-pyridyl, or 2-furyl;
or a pharmaceutically acceptable salt, solvate, prodrug, or pharmaceutically active metabolite thereof.

8. A method according to claim 1 utilizing a rhinovirus inhibitor of the formula III: 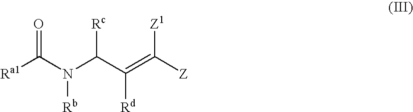
wherein:
R^{a1} is a cycloalkyl, heterocycloalkyl, aryl or heteroaryl group, provided that R^{a1} is not a substituted pyrrolidinyl, where the cycloalkyl, heterocycloalkyl, aryl or heteroaryl group is unsubstituted or substituted with one or more suitable substituents;
R^{c} is a substituent having the formula: 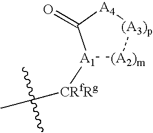
wherein:
R^{f} and R^{g} are each independently H or lower alkyl;
m is 0 or 1;
p is an integer of from 0 to 5;
A₁ is CH or N;
when p is 1, 2, 3, 4, or 5, A₂ is C(R^{h})(Rⁱ), N(R^{j}), S, S(O), S(O)₂, or O, and when p is 0, A₂ is C(R^{h})(Rⁱ)(R^{j}), N(Rⁱ)(R^{j}), S(Rⁱ), S(O)(Rⁱ), S(O)₂(Rⁱ), or O(Rⁱ), where each R^{h}, Rⁱ and R^{j} is independently H or a lower alkyl group;
each A₃ present is independently C(R^{h})(Rⁱ), N(R^{j}), S, S(O), S(O)₂, or O; where each R^{h}, Rⁱ and R^{j} is independently H or lower alkyl;
when p is 1, 2, 3, 4, or 5, A₄ is N(R^{k}), C(R^{h})(Rⁱ), or O; and when p is 0, A₄ is N(R^{k})(Rⁱ), C(R^{h})(Rⁱ)(R^{j}), and O(Rⁱ), where each R^{h}, Rⁱ and R^{j} is independently H or lower alkyl, each R^{k} is H, alkyl, aryl, or acyl, and each R^{l} is H, alkyl, or aryl;
provided that no more than two heteroatoms occur consecutively in the above-depicted ring formed by A₁, (A₂)ₘ, (A₃)ₚ, A₄, and C═O, where each dotted line in the ring depicts a single bond when A₂ is present and a hydrogen atom when A₂ is absent;
R^{d} is H, halogen, hydroxyl or an alkyl, alkoxy or alkylthio group, where the alkyl, alkoxy or alkylthio group is unsubstituted or substituted with one or more suitable substituents;
R^{b} is H or an alkyl group, unsubstituted or substituted with one or more suitable substituents;
Z and Z¹ are each independently H, F, an alkyl, cycloalkyl, heterocycloalkyl, aryl or heteroaryl group, where the alkyl, cycloalkyl, heterocycloalkyl, aryl or heteroaryl group is unsubstituted or substituted with one or more suitable substituents, —C(O)Rⁿ —CO₂Rⁿ —CN, —C(O)NRⁿR^{o}, —C(O)NRⁿOR^{o}, —C(S)Rⁿ, —C(S)ORⁿ —C(S)NRⁿR^{o}, —C(═NRⁿ)R^{o}, —C(═NRⁿ)OR^{o}, —NO₂, —SOR^{o}, —SO₂Rⁿ, —SO₂NRⁿR^{o}, —SO₂(NRⁿ)(OR^{o}), —SONRⁿ, —SO₃Rⁿ, —PO(ORⁿ)₂, —PO(ORⁿ)(OR^{o}), —PO(NRⁿR^{o})(OR^{p}), —PO(NRⁿR^{o})(NR^{p}R^{q}), —C(O)NRⁿNR^{o}R^{p}, —C(S)NRⁿNR^{o}R^{p}, where Rⁿ, R^{o}, R^{p} and R^{q} are each independently H or an alkyl, cycloalkyl, aryl, heterocycloalkyl, acyl or thioacyl group, where the alkyl, cycloalkyl, aryl, heterocycloalkyl, acyl or thioacyl group is unsubstituted or substituted with one or more suitable substituents, or where any two of the Rⁿ, R^{o}, R^{p} and R^{q}, taken together with the atoms to which they are bonded, form a heterocycloalkyl group, which may be optionally substituted,
or Z and R^{d},together with the atoms to which they are bonded, form a cycloalkyl or heterocycloalkyl group, where Z and R^{d} are as defined above except for moieties that cannot form the cycloalkyl or heterocycloalkyl group,
or Z and Z¹, together with the atoms to which they are bonded, form a cycloalkyl or heterocycloalkyl group, where Z and Z¹ are as defined above (except for moieties that cannot form the cycloalkyl or heterocycloalkyl group);
or a prodrug, pharmaceutically acceptable salt, pharmaceutically active metabolite, or pharmaceutically acceptable solvate thereof.

9. A method according to claim 1 utilizing a rhinovirus inhibitor of the formula IIIA: 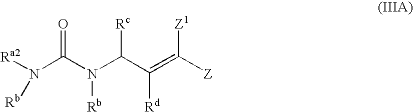
wherein:
R^{a2} is an alkyl, aryl or heteroaryl group, where the alkyl, aryl or heteroaryl group is unsubstituted or substituted with one or more suitable substituents; and
R^{c} is a substituent having the formula: 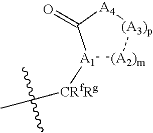
wherein:
R^{f} and R^{g} are each independently H or lower alkyl;
m is 0 or 1;
p is an integer of from 0 to 5;
A₁ is CH or N;
when p is 1, 2, 3, 4, or 5, A₂ is C(R^{h})(Rⁱ), N(R^{j}), S, S(O), S(O)₂, or O, and when p is 0, A₂ is C(R^{h})(Rⁱ)(R^{j}), N(Rⁱ)(R^{j}), S(Rⁱ), S(O)(Rⁱ), S(O)₂(Rⁱ), or O(Rⁱ), where each R^{h}, Rⁱ and R^{j} is independently H or a lower alkyl group;
each A₃ present is independently C(R^{h})(Rⁱ), N(R^{j}), S, S(O), S(O)₂, or O; where each R^{h}, Rⁱ and R^{j} is independently H or lower alkyl;
when p is 1, 2, 3, 4, or 5, A₄ is N(R^{k}), C(R^{h})(Rⁱ), or O; and when p is 0, A₄ is N(R^{k})(Rⁱ), C(R^{h})(Rⁱ)(R^{j}), and O(Rⁱ), where each R^{h}, Rⁱ and R^{j} is independently H or lower alkyl, each R^{k} is H, alkyl, aryl, or acyl, and each R^{l} is H, alkyl, or aryl;
provided that no more than two heteroatoms occur consecutively in the above-depicted ring formed by A₁, (A₂)ₘ, (A₃)ₚ, A₄, and C═O, where each dotted line in the ring depicts a single bond when A₂ is present and a hydrogen atom when A₂ is absent;
R^{d} is H, halogen, hydroxyl or an alkyl, alkoxy or alkylthio group, where the alkyl, alkoxy or alkylthio group is unsubstituted or substituted with one or more suitable substituents;
R^{b} is H or an alkyl group, unsubstituted or substituted with one or more suitable substituents;
Z and Z¹ are each independently H, F, an alkyl, cycloalkyl, heterocycloalkyl, aryl or heteroaryl group, where the alkyl, cycloalkyl, heterocycloalkyl, aryl or heteroaryl group is unsubstituted or substituted with one or more suitable substituents, —C(O)Rⁿ —CO₂Rⁿ —CN, —C(O)NRⁿR^{o}, —C(O)NR OR^{o}, —C(S)Rⁿ, —C(S)ORⁿ —C(S)NRⁿR^{o}, —C(═NRⁿ)R^{o}, —C(═NRⁿ)OR^{o}, —NO₂, —SOR^{o}, —SO₂Rⁿ, —SO₂NRⁿR^{o}, —SO₂(NRⁿ)(OR^{o}), —SONRⁿ, —SO₃Rⁿ, —PO(ORⁿ)₂, —PO(ORⁿ)(OR^{o}), —PO(NRⁿR^{o})(OR^{p}), —PO(NRⁿR^{o})(NR^{p}R^{q}), —C(O)NRⁿNR^{o}R^{p}, —C(S)NRⁿNR^{o}R^{p}, where Rⁿ, R^{o}, R^{p} and R^{q} are each independently H or an alkyl, cycloalkyl, aryl, heterocycloalkyl, acyl or thioacyl group, where the alkyl, cycloalkyl, aryl, heterocycloalkyl, acyl or thioacyl group is unsubstituted or substituted with one or more suitable substituents, or where any two of the Rⁿ, R^{o}, R^{p} and R^{q}, taken together with the atoms to which they are bonded, form a heterocycloalkyl group, which may be optionally substituted,
or Z and R^{d}, together with the atoms to which they are bonded, form a cycloalkyl or heterocycloalkyl group, where Z and R^{d} are as defined above except for moieties that cannot form the cycloalkyl or heterocycloalkyl group,
or Z and Z¹, together with the atoms to which they are bonded, form a cycloalkyl or heterocycloalkyl group, where Z and Z¹ are as defined above (except for moieties that cannot form the cycloalkyl or heterocycloalkyl group);
or a prodrug, pharmaceutically acceptable salt, pharmaceutically active metabolite, or pharmaceutically acceptable solvate thereof.

10. A method according to claim 1 utilizing a rhinovirus inhibitor of the formula IIIB: 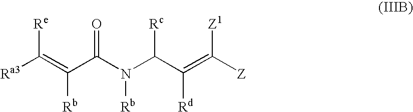
wherein:
R^{a3} is an aryl, heterocycloalkyl, heteroaryl or arylaminocarbonyl group, where the aryl, heterocycloalkyl, heteroaryl or arylaminocarbonyl group is unsubstituted or substituted with one or more suitable substituents; and
R^{c} is a substituent having the formula: 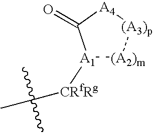
wherein:
R^{f} and R^{g} are each independently H or lower alkyl;
m is 0 or 1;
p is an integer of from 0 to 5;
A₁ is CH or N;
when p is 1, 2, 3, 4, or 5, A₂ is C(R^{h})(Rⁱ), N(R^{j}), S, S(O), S(O)₂, or O, and when p is 0, A₂ is C(R^{h})(Rⁱ)(R^{j}), N(Rⁱ)(Rⁱ), S(Rⁱ), S(O)(Rⁱ), S(O)₂(Rⁱ), or O(Rⁱ), where each R^{h}, Rⁱ and R^{j} is independently H or a lower alkyl group;
each A₃ present is independently C(R^{h})(Rⁱ), N(R^{j}), S, S(O), S(O)₂, or O; where each R^{h}, Rⁱ and R^{j} is independently H or lower alkyl;
when p is 1, 2, 3, 4, or 5, A₄ is N(R^{k}), C(R^{h})(Rⁱ), or O; and when p is O, A₄ is N(R^{k})(Rⁱ), C(R^{h})(Rⁱ)(R^{j}), and O(Rⁱ), where each R^{h}, Rⁱ and R^{j} is independently H or lower alkyl, each R^{k} is H, alkyl, aryl, or acyl, and each R^{l} is H, alkyl, or aryl;
provided that no more than two heteroatoms occur consecutively in the above-depicted ring formed by A₁, (A₂)ₘ, (A₃)ₚ, A₄, and C═O, where each dotted line in the ring depicts a single bond when A₂ is present and a hydrogen atom when A₂ is absent;
R^{d} is H, halogen, hydroxyl or an alkyl, alkoxy or alkylthio group, where the alkyl, alkoxy or alkylthio group is unsubstituted or substituted with one or more suitable substituents;
R^{b} is H or an alkyl group, unsubstituted or substituted with one or more suitable substituents;
R^{e} is H, halogen, hydroxyl or an alkyl, alkoxy or alkylthio group, where the alkyl, alkoxy or alkylthio group is unsubstituted or substituted with one or more suitable substituents;
Z and Z¹ are each independently H, F, an alkyl, cycloalkyl, heterocycloalkyl, aryl or heteroaryl group, where the alkyl, cycloalkyl, heterocycloalkyl, aryl or heteroaryl group is unsubstituted or substituted with one or more suitable substituents, —C(O)Rⁿ —CO₂Rⁿ —CN, —C(O)NRⁿR^{o}, —C(O)NRⁿOR^{o}, —C(S)Rⁿ, —C(S)ORⁿ —C(S)NRⁿR^{o}, —C(═NRⁿ)R^{o}, —C(═NRⁿ)OR^{o}, —NO₂, —SOR^{o}, —SO₂Rⁿ, —SO₂NRⁿR^{o}, —SO₂(NRⁿ)(OR^{o}), —SONRⁿ, —SO₃Rⁿ, —PO(ORⁿ)₂, —PO(ORⁿ)(OR^{o}), —PO(NRⁿR^{o})(OR^{o}), —PO(NRⁿR^{o})(NR^{p}R^{o}) —C(O)NRⁿNR^{o}R^{p}, —C(S)NRⁿNR^{o}R^{p}, where Rⁿ, R^{o}, R^{p} and R^{q} are each independently H or an alkyl, cycloalkyl, aryl, heterocycloalkyl, acyl or thioacyl group, where the alkyl, cycloalkyl, aryl, heterocycloalkyl, acyl or thioacyl group is unsubstituted or substituted with one or more suitable substituents, or where any two of the Rⁿ, R^{o}, R^{p} and R^{q}, taken together with the atoms to which they are bonded, form a heterocycloalkyl group, which may be optionally substituted,
or Z and R^{d},together with the atoms to which they are bonded, form a cycloalkyl or heterocycloalkyl group, where Z and R^{d} are as defined above except for moieties that cannot form the cycloalkyl or heterocycloalkyl group,
or Z and Z¹, together with the atoms to which they are bonded, form a cycloalkyl or heterocycloalkyl group, where Z and Z¹ are as defined above (except for moieties that cannot form the cycloalkyl or heterocycloalkyl group);
or a prodrug, pharmaceutically acceptable salt, pharmaceutically active metabolite, or pharmaceutically acceptable solvate thereof.

11. A method according to claim 1 utilizing a rhinovirus inhibitor of the formula IIIC: 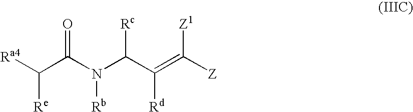
wherein:
R^{a4} is an aryloxy, heteroaryloxy, alkyloxy, cycloalkyloxy, heterocycloalkyloxy, aryl, cycloalkyl, or heteroaryl group, where the aryloxy, heteroaryloxy, alkyloxy, cycloalkyloxy, heterocycloalkyloxy, aryl, cycloalkyl, or heteroaryl group is unsubstituted or substituted with one or more suitable substituents; and
R^{c} is a substituent having the formula: 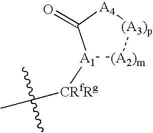
wherein:
R^{f} and R^{g} are each independently H or lower alkyl;
m is 0 or 1;
p is an integer of from 0 to 5;
A₁ is CH or N;
when p is 1, 2, 3, 4, or 5, A₂ is C(R^{h})(Rⁱ), N(R^{j}), S, S(O), S(O)₂, or O, and when p is 0, A₂ is C(R^{h})(Rⁱ)(R^{j}), N(Rⁱ)(R^{j}), S(Rⁱ), S(O)(RI), S(O)₂(Rⁱ), or O(Rⁱ), where each R^{h}, Rⁱ and R^{j} is independently H or a lower alkyl group;
each A₃ present is independently C(R^{h})(Rⁱ), N(Rⁱ), S, S(O), S(O)₂, or O; where each R^{h}, R and R¹ is independently H or lower alkyl;
when p is 1, 2, 3, 4, or 5, A₄ is N(R^{k}), C(R^{h})(Rⁱ), or O; and when p is 0, A₄ is N(R^{k})(Rⁱ), C(R^{h})(Rⁱ)(R^{j}), and O(Rⁱ), where each R^{h}, Rⁱ and R^{j} is independently H or lower alkyl, each R^{k} is H, alkyl, aryl, or acyl, and each R^{l} is H, alkyl, or aryl;
provided that no more than two heteroatoms occur consecutively in the above-depicted ring formed by A₁, (A₂)ₘ, (A₃)ₚ, A₄, and C═O, where each dotted line in the ring depicts a single bond when A₂ is present and a hydrogen atom when A₂ is absent;
R^{d} is H, halogen, hydroxyl or an alkyl, alkoxy or alkylthio group, where the alkyl, alkoxy or alkylthio group is unsubstituted or substituted with one or more suitable substituents;
R^{b} is H or an alkyl group, unsubstituted or substituted with one or more suitable substituents;
R^{e} is H, halogen, hydroxyl or an alkyl, alkoxy or alkylthio group, where the alkyl, alkoxy or alkylthio group is unsubstituted or substituted with one or more suitable substituents;
Z and Z¹ are each independently H, F, an alkyl, cycloalkyl, heterocycloalkyl, aryl or heteroaryl group, where the alkyl, cycloalkyl, heterocycloalkyl, aryl or heteroaryl group is unsubstituted or substituted with one or more suitable substituents, —C(O)Rⁿ —CO₂Rⁿ —CN, —C(O)NRⁿR^{o}, —C(O)NRⁿOR^{o}, —C(S)Rⁿ, —C(S)ORⁿ —C(S)NRⁿR^{o}, —C(═NRⁿ)R^{o}, —C(═NRⁿ)OR^{o}, —NO₂, —SOR^{o}, —SO₂Rⁿ, —SO₂NRⁿR^{o}, —SO₂(NRⁿ)(OR^{o}), —SONRⁿ, —SO₃Rⁿ, —PO(ORⁿ)₂, —PO(ORⁿ)(OR^{o}), —PO(NRⁿR^{o})(OR^{p}, —PO(NRⁿR^{o})(NR^{p}R^{q}), —C(O)NRⁿNR^{o}R^{p}, —C(S)NRⁿNR^{o}R^{p}, where Rⁿ, R°, R^{p} and R^{q} are each independently H or an alkyl, cycloalkyl, aryl, heterocycloalkyl, acyl or thioacyl group, where the alkyl, cycloalkyl, aryl, heterocycloalkyl, acyl or thioacyl group is unsubstituted or substituted with one or more suitable substituents, or where any two of the Rⁿ, R^{o}, R^{p} and R^{q} taken together with the atoms to which they are bonded, form a heterocycloalkyl group, which may be optionally substituted,
or Z and R^{d}, together with the atoms to which they are bonded, form a cycloalkyl or heterocycloalkyl group, where Z and R^{d} are as defined above except for moieties that cannot form the cycloalkyl or heterocycloalkyl group,
or Z and Z¹, together with the atoms to which they are bonded, form a cycloalkyl or heterocycloalkyl group, where Z and Z¹ are as defined above (except for moieties that cannot form the cycloalkyl or heterocycloalkyl group);
or a prodrug, pharmaceutically acceptable salt, pharmaceutically active metabolite, or pharmaceutically acceptable solvate thereof.

12. A method according to claim 1 utilizing a rhinovirus inhibitor of the formula IV: 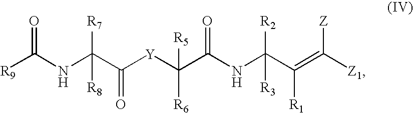
wherein:
Y is —N(R_{y})—, —C(R_{y})(R_{y})—, or —O—, where each R_{y} is independently H or lower alkyl;
R₁ is H, F, an alkyl group, OH, SH, or an O-alkyl group;
R₂ and R₃ are each independently H; 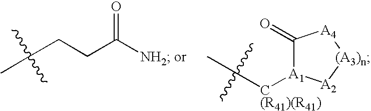
where n is an integer from 0 to 5, A₁ is CH or N, A₂ and each A₃ are independently selected from C(R₄₁)(R₄₁), N(R₄₁), S, S(O), S(O)₂, and O, and A₄ is NH or NR₄₁, where each R₄₁ is independently H or lower alkyl, provided that no more than 2 heteroatoms occur consecutively in the ring formed by A₁, A₂, (A₃)ₙ, A₄ and C═O; and provided that at least one of R₂ and R₃ is 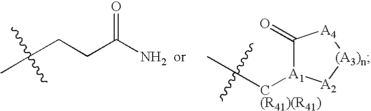
R₅ and R₆ are each independently H, F, an alkyl group, a cycloalkyl group, a heterocycloalkyl group, an aryl group, or a heteroaryl group;
R₇ and R₈ are each independently H, an alkyl group, a cycloalkyl group, a heterocycloalkyl group, an aryl group, a heteroaryl group, —OR₁₇, —SR₁₇, —NR₁₇R₁₈, —NR₁₉NR₁₇R₁₈, or —NR₁₇OR₁₈, where R₁₇, R₁₈, and R₁₉ are each independently H, an alkyl group, a cycloalkyl group, a heterocycloalkyl group, an aryl group, a heteroaryl group, or an acyl group;
R^{g} is a five-membered heterocycle having from one to three heteroatoms selected from O, N, and S, or R₉ is 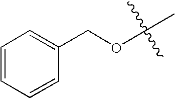
where R₂ is 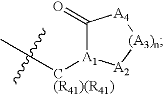
and
Z and Z₁ are each independently H, F, an alkyl group, a cycloalkyl group, a heterocycloalkyl group, an aryl group, a heteroaryl group, —C(O)R₂₁, —CO₂R₂₁, —CN, —C(O)NR₂₁,R₂₂, —C(O)NR₂₁OR₂₂, —C(S)R₂₁, —C(S)NR₂₁R₂₂, —NO₂, —SOR₂₁, —SO₂R₂₁, —SO₂NR₂₁R₂₂, —SO(NR₂,)(OR₂₂), —SONR₂₁, —SO₃R₂₁, —PO(OR₂₁)₂, —PO(R₂₁)(R₂₂), —PO(NR₂₁R₂₂)(OR₂₃), PO(NR₂₁R₂₂)(NR₂₃R₂₄), —C(O)NR₂,NR₂₂R₂₃, or —C(S)NR₂,NR₂₂R₂₃, where R₂₁, R₂₂, R₂₃, and R₂₄ are each independently H, an alkyl group, a cycloalkyl group, a heterocycloalkyl group, an aryl group, a heteroaryl group, an acyl group, or a thioacyl group, or any two of R₂₁, R₂₂, R₂₃, and R₂₄, together with the atom(s) to which they are bonded, form a heterocycloalkyl group, provided that Z and Z₁ are not both H;
or Z₁ and R₁, together with the atoms to which they are bonded, form a cycloalkyl or heterocycloalkyl group;
or Z and Z₁, together with the atoms to which they are bonded, form a cycloalkyl or heterocycloalkyl group;
or a prodrug, pharmaceutically active metabolite, pharmaceutically acceptable salt, or solvate thereof.

13. A method according to claim 1 utilizing a rhinovirus inhibitor of the formula V: 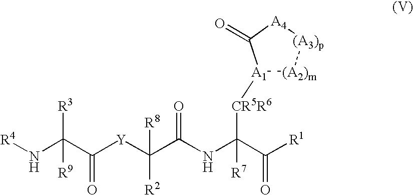
wherein:
Y is —N(R^{y})—, —C(R^{y})(R^{y})—, or —O—, where each R^{y} is independently H or lower alkyl;
R¹ is selected from optionally substituted alkyl, cycloalkyl, heterocycloalkyl, aryl, heteroaryl, and —C(O)R¹⁶, where R¹⁶ is selected from optionally substituted alkyl, cycloalkyl, heterocycloalkyl, aryl, heteroaryl, alkoxy, cycloalkoxy, heterocycloalkoxy, aryloxy, heteroaryloxy, and amine;
R² and R⁸ are each independently selected from H, F, and optionally substituted alkyl, cycloalkyl, heterocycloalkyl, aryl, and heteroaryl;
R³ and R⁹ are each independently selected from H and optionally substituted alkyl, cycloalkyl, heterocycloalkyl, aryl, heteroaryl, —OR¹⁷, —SR¹⁷, —NR¹⁷R¹⁸, —NR¹⁹NR¹⁷R¹⁸, and —NR¹⁷OR¹⁸, where R¹⁷, R¹⁸, and R¹⁹ are each independently selected from H, alkyl, cycloalkyl, heterocycloalkyl, aryl, heteroaryl, and acyl;
R⁴is a suitable organic moiety;
each of R⁵, R⁶ and R⁷is independently H, F, or lower alkyl;
m is 0 or 1;
p is 0, 1, 2, 3, 4, or 5;
A₁ is CH or N;
when m is 1, A₂ is selected from C(R¹⁰)(R¹¹), N(R¹²), S, S(O), S(O)₂, and O;
when p is not 0, each A₃ is independently selected from C(R¹⁰)(R¹¹), N(R¹²), S, S(O), S(O)₂, and O;
where R¹⁰, R¹¹ and R¹² are each independently H or lower alkyl;
when p is not 0, A₄ is selected from N(R¹³), C(R¹⁰)(R¹¹), and O, and when p is 0, A₄ is selected from N(R¹³)(R¹⁴), C(R¹⁰)(R¹¹)(R¹²), and O(R¹⁴), provided that when A₄ is O(R¹⁴), A₁ is not CH; where R¹⁰,
R¹¹ and R¹² are each independently H or lower alkyl, R¹³ is H, alkyl, aryl, or acyl, and R¹⁴ is H, alkyl, or aryl;
provided that A₁, (A₂)ₘ, (A₃)ₚ, and A₄ together do not include more than two consecutive heteroatoms;
or a prodrug, pharmaceutically acceptable salt, pharmaceutically active metabolite, or pharmaceutically acceptable solvate thereof.

14. A method according to claim 1 utilizing a rhinovirus inhibitor of the formula VI: 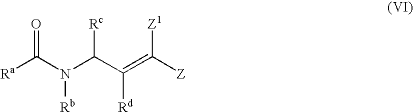
wherein:
R^{a} is an alkylcarbonylalkyl, cycloalkylcarbonylalkyl, arylcarbonylalkyl, heteroarylcarbonylalkyl, alkylcarbonylaminoalkyl, cycloalkylcarbonylaminoalkyl, heterocycloalkylcarbonylaminoalkyl, arylcarbonylaminoalkyl, heteroarylcarbonylaminoalkyl, alkylaminocarbonylalkyl, cycloalkylaminocarbonylalkyl, heterocycloalkylaminocarbonylalkyl, arylaminocarbonylalkyl, heteroarylaminocarbonylalkyl group, where each alkyl, cycloalkyl, heterocycloalkyl, aryl and heteroaryl moiety thereof may be unsubstituted or substituted with one or more suitable substituents;
R^{b} is H or an alkyl group, unsubstituted or substituted with one or more suitable substituents;
R^{d} is H, halo, hydroxyl, or an alkyl, alkoxy or alkylthio group, where the alkyl, alkoxy or alkylthio group is unsubstituted or substituted with one or more suitable substituents;
R^{c} is a moiety having the formula: 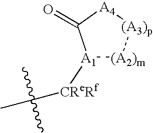
R^{e} and R^{f} are each independently H or a lower alkyl group;
m is 0 or 1, provided that when m is 1, R^{a} is not an amino-substituted alkylcarbonylalkyl or amino-substituted alkylcarbonylaminoalkyl group, and when m is 0, R^{a} is selected from an alkylaminocarbonylalkyl, cycloalkylaminocarbonylalkyl, heterocycloalkylaminocarbonylalkyl, arylaminocarbonylalkyl, heteroarylaminocarbonylalkyl and heteroarylcarbonylaminoalkyl group, provided that R^{a} is not substituted indolecarbonylaminoalkyl;
p is an integer of from 0 to 5;
A₁ is CH or N;
when p is 1, 2, 3, 4, or 5, A₂ is C(R^{g})(R^{h}), N(Rⁱ), S, S(O), S(O)₂, or O, and when p is 0, A₂ is C(R^{g})(R^{h})(Rⁱ), N(R^{g})(Rⁱ), S(R^{g}), S(O)(R^{g}), S(O)₂(R^{g}), or O(R^{g}), where each R^{g}, R^{h} and Rⁱ is independently H or a lower alkyl group;
each A₃ present is independently C(R^{g})(R^{h}), N(Rⁱ), S, S(O), S(O)₂, or O, where each R^{g}, R^{h} and Rⁱ is independently H or a lower alkyl group;
when p is 1, 2, 3, 4, or 5, A₄ is N(R^{j}), C(R^{g})(R^{h}), or O, and when p is 0, A₄ is N(R^{j})(R^{k}), C(R^{g})(R^{h})(Rⁱ), and O(R^{k}), where each R^{g}, R^{h} and Rⁱ is independently H or a lower alkyl group, each R^{j} is H, an alkyl, aryl, or acyl group, and each R^{k} is H or an alkyl or aryl group;
provided that no more than two heteroatoms occur consecutively in the above-depicted ring formed by A₁, (A₂)ₘ, (A₃)ₚ, A₄, and C═O, where each dotted line in the ring depicts a single bond when A₂ is present and a hydrogen atom when A₂ is absent; and
Z and Z¹ are each independently H, F, an alkyl, cycloalkyl, heterocycloalkyl, aryl or heteroaryl group, where the alkyl, cycloalkyl, heterocycloalkyl, aryl or heteroaryl group is unsubstituted or substituted with one or more suitable substituents, —C(O)R^{l}, —CO₂R^{l}, —CN, —C(O)NR^{l}R^{m}, —C(O)NR^{l}OR^{m}, —C(S)R^{l}, —C(S)OR^{l} —C(S)NR^{l}R^{m}, —C(═NR^{l})R^{m}, —C(═NR^{l})OR^{m}, —NO₂, —SOR^{m}, —SO₂R^{l}, —SO₂NR^{l}R^{m}, —SO₂(NR^{l})(OR^{m}), —SONR^{l}, —SO₃R^{l}, —PO(OR^{l})₂, —PO(OR^{l})(OR^{m}), —PO(NR^{l}R^{m})(ORⁿ), —PO(NR^{l}R^{m})(NRⁿR^{o}), —C(O)NR^{l}NR^{m}Rⁿ, —C(S)NR^{l}NR^{m}Rⁿ, where R^{l}, R^{m}, Rⁿ and R^{o} are each independently H or an alkyl, cycloalkyl, aryl, heterocycloalkyl, acyl or thioacyl group, where the alkyl, cycloalkyl, aryl, heterocycloalkyl, acyl or thioacyl group is unsubstituted or substituted with one or more suitable substituents, or where any two of the R^{l}, R^{m}, Rⁿ and R^{o}, taken together with the atoms to which they are bonded, form a heterocycloalkyl group, which may be optionally substituted,
or Z and R^{d}, together with the atoms to which they are bonded, form a cycloalkyl or heterocycloalkyl group, where Z and R^{d} are as defined above except for moieties that cannot form the cycloalkyl or heterocycloalkyl group,
or Z and Z¹, together with the atoms to which they are bonded, form a cycloalkyl or heterocycloalkyl group, where Z and Z¹ are as defined above;
or a prodrug, pharmaceutically acceptable salt, pharmaceutically active metabolite, or pharmaceutically acceptable solvate of said compound.

15. A method according to claim 1 utilizing a rhinovirus inhibitor of the formula VIIA: 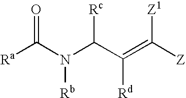
wherein:
R^{a} is substituted or unsubstituted heterocycloalkyl or heterocycloalkylalkyl;
R^{b} is a substituent having the formula: 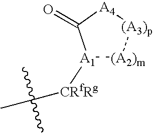
wherein:
R^{f} and R^{g} are independently H or lower alkyl;
m is 1;
p is an integer of from 1 to 5;
A₁ is CH or N;
A₂ is C(R^{h})(Rⁱ), N(R^{j}), S, S(O), S(O)₂, or O; where each R^{h}, Rⁱ and R^{j} is independently H or lower alkyl;
each A₃ present is independently C(R^{h})(Rⁱ), N(R^{j}), S, S(O), S(O)₂, or O; where each R^{h}, Rⁱ and R^{j} is independently H or lower alkyl;
A₄ is N(R^{k}), C(R^{h})(Rⁱ), or O;
provided that no more than two heteroatoms occur consecutively in the above-depicted ring formed by A₁, (A₂)ₘ, (A₃)ₚ, A₄, and C═O, where each dotted line in the ring depicts a single bond;
R^{c} is H, halogen or a substituted or unsubstituted lower alkyl group;
R^{d} is H, halogen, hydroxyl, a substituted or unsubstituted alkyl, alkoxy or alkylthio group;
R^{e} is H or a substituted or unsubstituted alkylgroup; and
Z and Z¹ are independently H, F, a unsubstituted or substituted alkyl group, cycloalkyl group, heterocycloalkyl group, aryl group or heteroaryl group, —C(O)Rⁿ, —CO₂Rⁿ, —CN, —C(O)NRⁿR^{o}, —C(O)NRⁿOR^{o}, —C(S)Rⁿ, —C(S)ORⁿ, —C(S)NRⁿR^{o}, —NO₂, —SOR^{o}, —SO₂Rⁿ, —SO₂NRⁿR^{o}, —SO₂(NRⁿ)(OR^{o}), —SONRⁿ, —SO₃Rⁿ, —PO(ORⁿ)₂, —PO(ORⁿ)(OR^{o}) —PO(NRⁿR^{o})(OR^{p}) —PO(NRⁿR^{o})(NR^{p}R^{q}), —C(O)NRⁿNR^{o}R^{p}, or —C(S)NRNR^{o}R^{p}, wherein Rⁿ, R^{o}, R^{p} and R^{q} are independently H, a substituted or unsubstituted alkyl group, cycloalkyl group, aryl group, heterocycloalkyl group, acyl group or thioacyl group, or wherein any two of the Rⁿ, R^{o}, R^{p} and R^{q}, taken together with the atoms to which they are bonded, form a heterocycloalkyl group, which may be optionally substituted,
or Z and R^{d}, together with the atoms to which they are bonded, form a cycloalkyl or heterocycloalkyl group,
or Z and Z¹, together with the atom to which they are bonded, form a cycloalkyl or heterocycloalkyl group;
or a prodrug, pharmaceutically acceptable salt, or pharmaceutically acceptable solvate thereof.
